(19) United States
(12) Patent Application Publication
(10) Pub. No.: US 2016/0376652 A1
(43) Pub. Date: Dec. 29, 2016

(54) COMPOSITIONS AND METHODS FOR DIAGNOSIS AND PREDICTION OF SOLID ORGAN GRAFT REJECTION

(71) Applicant: IMMUCOR GTI DIAGNOSTICS, INC., Norcross, GA (US)

(72) Inventor: Minnie M. Sarwal, Mountain View, CA (US)

(21) Appl. No.: 14/916,639

(22) PCT Filed: Sep. 5, 2014
(86) PCT No.: PCT/US14/54309

§ 371 Date: Mar. 4, 2016

Related U.S. Application Data
(60) Provisional application No. 61/874,981, filed on Sep. 6, 2013.

(51) Int. Cl. C12Q 1/68

(52) U.S. Cl. CPC C12Q 1/6883; C12Q 2600/158; C12Q 2600/16

(57) ABSTRACT

Provided herein are methods, compositions, systems, and kits for diagnosing acute rejection of solid organ transplants using at least 5 genes selected from a 10-gene panel.

### CROSS-REFERENCE TO RELATED APPLICATION

This application claims the priority benefit to U.S. Provisional Patent Application Ser. No. 61/874,981 filed Sep. 6, 2013 the entire content of which is incorporated herein by reference.

### FIELD OF THE INVENTION

This disclosure relates to methods, compositions, systems and/or kits for the assessment of acute rejection of solid organ transplants. Provided herein are methods, compositions, systems, and kits for diagnosing acute rejection of solid organ transplants using at least 5 genes selected from a 10-gene panel.

### BACKGROUND OF THE INVENTION

Organ transplantation from a donor to a host recipient is a feature of certain medical procedures and treatment regimes. Following transplantation, immunosuppressive therapy is typically provided to the host recipient in order to maintain viability of the donor organ and to avoid graft rejection. When organ transplant rejection occurs, the response is typically classified as a hyperacute rejection, an acute rejection, or a chronic rejection. Hyperacute rejection occurs within minutes to hours following organ transplantation due to antibodies in the recipient's blood stream that react with the new organ, and is characterized by widespread glomerular capillary thrombosis and necrosis. Acute rejection (AR) generally occurs in the first 6 to 12 months following organ transplantation, and is a complex immune response that involves T-cell recognition of alloantigen in the graft and an inflammatory response within the graft itself. Chronic rejection is less well-defined than either hyperacute or acute rejection, and is likely due to both antibodies and lymphocytes.

Despite advances in immunosuppressive therapies and transplantation procedures, graft rejection is still a common risk in organ transplant recipients. For example, despite improvements in immunosuppressive therapy over the years, approximately 30-40% of heart transplant recipients require treatment for AR in the first year after transplantation (see Taylor et al., J Heart Transplant., 2009, 28(10):1007-22). Furthermore, AR remains a risk factor for graft dysfunction, mortality, and the development of cardiac allograft vasculopathy (CAV), which is the main cause of late graft failure (see Raichlin et al., J Heart Lung Transplant, 2009, 28(4):320-7).

Early detection of AR is one of the major clinical concerns in the care of transplant recipients, including recipients of solid organs such as heart, liver, lung, kidney, and intestines. Detection of AR before the onset of organ dysfunction allows successful treatment of AR with aggressive immunosuppression. It is equally important to reduce immunosuppression in patients who do not have AR to minimize drug toxicity. However, for most organs, rejection can only be unequivocally established by performing a biopsy of that organ. For example, the current definitive diagnosis of cardiac allograft rejection relies on the endomyocardial biopsy (EMB), an expensive, invasive, and inconvenient procedure. Most heart transplant recipients undergo routine EMB procedures up to 15 times in the first year, and more frequently if rejection is detected. This procedure, however, is limited by sampling error and interobserver variability (see Deng et al., Am J Transplant., 2006, 6(1):150-60; Wong et al., Cardiovasc Pathol., 2005, 14(4):176-80). Potential complications include arterial puncture, vasovagal reactions and prolonged bleeding during catheter insertion, arrhythmias and conduction abnormalities, pneumothorax, biopsy-induced tricuspid regurgitation, and even cardiac perforation (see Baraldi-Junkins et al., J Heart Lung Transplant, 1993, 12(1 Pt 1):63-7; Deckers et al., J Am Coll Cardiol., 1992, 19(1):43-7; Navia et al., J Heart Valve Dis., 2005, 14(2):264-7).

Although the diagnosis of acute rejection can be difficult, detecting immune-related injury in a timely fashion is crucial to ensuring graft health and long-term survival. A noninvasive biomarker panel for acute rejection that allows frequent immunologic monitoring of the graft would be of considerable value (see Evans et al., Am J Transplant., 2005, 5(6):1553-8; Mehra et al., Nat Clin Pract Cardiovasc Med., 2006, 3(3):136-43). Recently, a highly sensitive and specific gene-based biomarker panel was developed for diagnosis and prediction of biopsy confirmed acute renal transplant rejection (see Li et al., Am J Transplant., 2012, 12(10):2710-8; Bromberg et al., Am J Transplant, 2012, 12(10):2573-4), which was independently validated in an randomized multicenter trial (see Chaudhuri et al., Pediatric Transplantation., 2012, 16(5):E183-7; Naesens et al., Am J Transplant., 2012, 12(10):2730-43). The diagnosis of acute rejection prior to development of histopathological changes can enable the optimization of immunosuppressive therapy to prevent progression to chronic allograft dysfunction (see Kienzl et al., Transplantation., 2009, 88(4):553-60).

A noninvasive assay that permits detection of acute graft rejection across different organs with high specificity (to reduce invasive protocol biopsies in patients with low risk of AR) and with high sensitivity (to increase clinical surveillance for patients at high risk of AR), earlier than is currently possible, would result in timely clinical intervention in order to mitigate AR, as well as to reduce the immunosuppression protocols for quiescent and stable patients. Many assays are likely to be dependent upon recipient age, co-morbidities, immunosuppression usage, and/or cause of end-stage renal disease. Therefore, there remains a need for systems and methods for predicting, diagnosing, and monitoring an AR response in a subject that has received an organ transplant.

All patents, patent applications, publications, documents, and articles cited herein are incorporated herein by reference in their entireties, unless otherwise stated.

### BRIEF SUMMARY OF THE INVENTION

Disclosed herein are methods, compositions, systems, and kits for assessing acute rejection in a subject who has a solid organ transplant, wherein detection of at least 5 genes selected from a 10-panel aids in, inter alia, predicting the likelihood of an acute rejection response, diagnosing an acute rejection response, identifying a subject at risk for an acute rejection response and monitoring the subject for an acute rejection response.

Accordingly, in one aspect, the invention described herein provides for methods for aiding in the diagnosis of an acute rejection response in a subject who has received a solid organ allograft, wherein the method comprises: a) detecting a gene expression level for at least ten genes in a sample from the subject, wherein the at least ten genes comprise CFLAR, DUSP1, IFNGR1, ITGAX, MAPK9, NAMPT, NKTR, PSEN1, RNF130, and RYBP; and b) comparing the gene expression level to a reference expression level of the at least ten genes, wherein a statistical difference or a statistical similarity between the gene expression level and the reference expression level of at least five genes selected from the group consisting of CFLAR, DUSP1, IFNGR1, ITGAX, MAPK9, NAMPT, NKTR, PSEN1, RNF130, and RYBP, thereby aiding in the diagnosis of an acute rejection response. In any of the embodiments herein, the reference expression level may be obtained from a control sample from at least one subject with an acute rejection response to a solid organ allograft. In any of the embodiments herein, the statistical similarity between the gene expression level and the reference expression level for the at least five genes may aid in the diagnosis of an acute rejection response in the subject. In any of the embodiments herein, the statistical difference between the gene expression level and the reference expression level for the at least five genes may aid in the diagnosis of the absence of an acute rejection response in the subject. In any of the embodiments herein, the reference expression level can be obtained from a control sample from at least one subject without an acute rejection response to a solid organ allograft. In any of the embodiments herein, the statistical similarity between the gene expression level and the reference expression level for the at least five genes may aid in the diagnosis of the absence of an acute rejection response in the subject. In any of the embodiments herein, the statistical difference between the gene expression level and the reference expression level for the at least five genes may aid in the diagnosis of an acute rejection response in the subject. In any of the embodiments herein, the sample may be a biological sample. In any of the embodiments herein, the biological sample can be selected from the group consisting of: a blood sample, a biopsy sample, a saliva sample, a cerebrospinal fluid sample, or a urine sample. In any of the embodiments herein, the biological sample may comprise peripheral blood leukocytes. In any of the embodiments herein, the biological sample may comprise peripheral blood mononuclear cells. In some of the embodiments herein, the biological sample is a bronchoalveolar lavage sample. In some of the embodiments herein, the biological sample is circulating nucleic acids or cell-free DNA or cell-free RNA. In any of the embodiments herein, the solid organ allograft can be one or more selected from the group consisting of: heart, lung, large intestine, small intestine, liver, kidney, pancreas, stomach, and bladder. In any of the embodiments herein, the step of detecting may comprise assaying the sample for an expression product of the at least ten genes. In some embodiments herein, the expression product is a nucleic acid transcript. In some embodiments herein, the expression product is a protein. In any of the embodiments herein, the step of detecting may comprise assaying the expression of the at least ten genes by hybridizing nucleic acids to oligonucleotide probes, by RT-PCR or by direct mRNA capture. In any of the embodiments herein, the step of detecting may comprise assaying the expression of the at least ten genes on one or more of: an array, a bead, and a nanoparticle. In some of the embodiments herein, the subject can have a cardiac acute rejection score of Grade 0, Grade 1A, Grade 1B, Grade 2, Grade 3A, Grade 3B, or Grade 4. In any of the embodiments herein, the comparing step may aid in the diagnosis of acute rejection with equal to or greater than 70% sensitivity. In any of the embodiments herein, the comparing step may aid in the diagnosis of acute rejection with equal to or greater than 70% specificity. In any of the embodiments herein, the comparing step may aid in the diagnosis of acute rejection with equal to or greater than 70% positive predictive value (ppv). In any of the embodiments herein, the comparing step may aid in the diagnosis of acute rejection with equal to or greater than 70% negative predictive value (npv).

In yet another aspect, the invention provides for methods for predicting the likelihood of an acute rejection response in a subject who has received a solid organ allograft, the method comprising: a) detecting a gene expression level for at least ten genes in a sample from the subject, wherein the at least ten genes comprise CFLAR, DUSP1, IFNGR1, ITGAX, MAPK9, NAMPT, NKTR, PSEN1, RNF130, and RYBP; and b) comparing the gene expression level to a reference expression level of the at least ten genes, wherein a statistical difference or a statistical similarity between the gene expression level and the reference expression level for at least five genes selected from the group consisting of CFLAR, DUSP1, IFNGR1, ITGAX, MAPK9, NAMPT, NKTR, PSEN1, RNF130, and RYBP, thereby predicting the likelihood of an acute rejection response in the subject. In some of the embodiments herein, the reference expression level is obtained from a control sample from at least one subject with an acute rejection response to a solid organ allograft. In some of the embodiments herein, the statistical similarity between the gene expression level and the reference expression level for the at least five genes predicts the likelihood of an acute rejection response in the subject. In some of the embodiments herein, the statistical difference between the gene expression level and the reference expression level for the at least five genes predicts the likelihood of the absence of an acute rejection response in the subject. In some of the embodiments herein, the reference expression level is obtained from a control sample from at least one subject without an acute rejection response to a solid organ allograft. In some of the embodiments herein, the statistical similarity between the gene expression level and the reference expression level for the at least five genes predicts the likelihood of the absence of an acute rejection response in the subject. In some of the embodiments herein, the statistical difference between the gene expression level and the reference expression level for the at least five genes predicts the likelihood of an acute rejection response in the subject. In any of the embodiments herein, the sample can be a biological sample. In any of the embodiments herein, the biological sample can be selected from the group consisting of: a blood sample, a biopsy sample, a saliva sample, a cerebrospinal fluid sample, or a urine sample. In any of the embodiments herein, the biological sample can comprises peripheral blood leukocytes. In any of the embodiments herein, the biological sample can comprises peripheral blood mononuclear cells. In any of the embodiments herein, the biological sample can be a bronchoalveolar lavage sample. In some of the embodiments herein, the biological sample is circulating nucleic acids or cell-free DNA or cell-free RNA. In any of the embodiments herein, the solid organ allograft can be one or more selected from the group consisting of: heart, lung, large intestine, small intestine, liver, kidney, pancreas, stomach, and bladder. In any of the embodiments herein, the step of detecting may comprise assaying the sample for an expression product of the at least ten genes. In any of the embodiments herein, the expression product can be a nucleic acid transcript. In any of the embodiments herein, the expression product can be a protein. In any of the embodiments herein, the step of detecting may comprise assaying the expression of the at least ten genes by hybridizing nucleic acids to oligonucleotide probes, by RT-PCR or by direct mRNA capture. In any of the embodiments herein, the step of detecting may comprise assaying the expression of the at least ten genes on one or more of: an array, a bead, and a nanoparticle. In some of the embodiments herein, the subject has a cardiac acute rejection score of Grade 0, Grade 1A, Grade 1B, Grade 2, Grade 3A, Grade 3B, or Grade 4. In some of the embodiments herein, the comparing step predicts the likelihood of acute rejection response with equal to or greater than 70% sensitivity. In some of the embodiments herein, the comparing step predicts the likelihood of acute rejection response with equal to or greater than 70% specificity. In some of the embodiments herein, the comparing step predicts the likelihood of acute rejection response with equal to or greater than 70% positive predictive value (ppv). In some of the embodiments herein, the comparing step predicts the likelihood of acute rejection response with equal to or greater than 70% negative predictive value (npv). In some of the embodiments herein, the expression level of the at least five genes is employed to predict the likelihood of an acute rejection response within 1 to 6 months of obtaining the sample.

In still another aspect, the invention provides for methods for monitoring the progression an acute rejection response in a subject who has received a solid organ allograft, the method comprising: a) detecting a gene expression level for at least ten genes in a sample from the subject, wherein the at least ten genes comprise CFLAR, DUSP1, IFNGR1, ITGAX, MAPK9, NAMPT, NKTR, PSEN1, RNF130, and RYBP; and b) comparing the gene expression level to a reference expression level of the at least ten genes; and c) determining whether the subject has an acute rejection response based upon a statistical difference or a statistical similarity between the gene expression level and the reference expression level of at least five genes selected from the group consisting of CFLAR, DUSP1, IFNGR1, ITGAX, MAPK9, NAMPT, NKTR, PSEN1, RNF130, and RYBP, thereby monitoring the progression of an acute rejection response in the subject. In some of the embodiments herein, the reference expression level is obtained from a control sample from at least one subject with an acute rejection response to a solid organ allograft. In some of the embodiments herein, the statistical similarity between the gene expression level and the reference expression level for the at least five genes determines the subject has an acute rejection response. In some of the embodiments herein, the statistical difference between the gene expression level and the reference expression level for the at least five genes determines the subject does not have an acute rejection response. In some of the embodiments herein, the reference expression level is obtained from a control sample from at least one subject without an acute rejection response to a solid organ allograft. In some of the embodiments herein, the statistical similarity between the gene expression level and the reference expression level for the at least five genes determines the subject does not have an acute rejection response. In some of the embodiments herein, the statistical difference between the gene expression level and the reference expression level for the at least five genes determines the subject has an acute rejection response. In any of the embodiments herein, the sample can be a biological sample. In any of the embodiments herein, the biological sample can be selected from the group consisting of: a blood sample, a biopsy sample, a saliva sample, a cerebrospinal fluid sample, or a urine sample. In any of the embodiments herein, the biological sample may comprise peripheral blood leukocytes. In any of the embodiments herein, the biological sample may comprise peripheral blood mononuclear cells. In any of the embodiments herein, the biological sample can be a bronchoalveolar lavage sample. In some of the embodiments herein, the biological sample is circulating nucleic acids or cell-free DNA or cell-free RNA. In any of the embodiments herein, the solid organ allograft can be one or more selected from the group consisting of: heart, lung, large intestine, small intestine, liver, kidney, pancreas, stomach, and bladder. In any of the embodiments herein, the step of detecting may comprise assaying the sample for an expression product of the at least ten genes. In any of the embodiments herein, the expression product may be a nucleic acid transcript. In any of the embodiments herein, the expression product can be a protein. In any of the embodiments herein, the step of detecting may comprise assaying the expression of the at least ten genes by hybridizing nucleic acids to oligonucleotide probes, by RT-PCR or by direct mRNA capture. In any of the embodiments herein, the step of detecting may comprise assaying the expression of the at least ten genes on one or more of: an array, a bead, and a nanoparticle. In some of the embodiments herein, the subject has an acute rejection score of Grade 0, Grade 1A, Grade 1B, Grade 2, Grade 3A, Grade 3B, or Grade 4. In some of the embodiments herein, the comparing step allows monitoring the progression of an acute rejection with equal to or greater than 70% sensitivity. In some of the embodiments herein, the comparing step allows monitoring the progression of an acute rejection with equal to or greater than 70% specificity. In some of the embodiments herein, the comparing step allows monitoring the progression of an acute rejection with equal to or greater than 70% positive predictive value (ppv). In some of the embodiments herein, the comparing step allows monitoring the progression of an acute rejection with equal to or greater than 70% negative predictive value (npv).

In another aspect, the invention provides for methods for identifying a subject who has received a solid organ allograft in need of treatment of an acute rejection response, the method comprising: a) detecting a gene expression level for at least ten genes in a sample from the subject, wherein the at least ten genes comprise CFLAR, DUSP1, IFNGR1, ITGAX, MAPK9, NAMPT, NKTR, PSEN1, RNF130, and RYBP; b) comparing the gene expression level to a reference expression level of the at least ten genes; and c) determining whether the subject has an acute rejection response based upon a statistical difference or a statistical similarity between the gene expression level and the reference expression level of at least five genes selected from the group consisting of CFLAR, DUSP1, IFNGR1, ITGAX, MAPK9, NAMPT, NKTR, PSEN1, RNF130, and RYBP, thereby identifying the subject in need of treatment of an acute rejection response. In some of the embodiments herein, the reference expression level is obtained from a control sample from at least one subject with an acute rejection response to a solid organ allograft. In some of the embodiments herein, the statistical similarity between the gene expression level and the reference expression level for the at least five genes identifies the subject in need of treatment for an acute rejection response. In some of the embodiments herein, the statistical difference between the gene expression level and the reference expression level for the at least five genes identifies the subject as not requiring treatment for an acute rejection response. In some of the embodiments herein, the reference expression level is obtained from a control sample from at least one subject without an acute rejection response to a solid organ allograft. In some of the embodiments herein, the statistical similarity between the gene expression level and the reference expression level for the at least five genes identifies the subject as not requiring treatment for an acute rejection response. In some of the embodiments herein, the statistical difference between the gene expression level and the reference expression level for the at least five genes identifies the subject in need of treatment for an acute rejection response. In any of the embodiments herein, the sample can be a biological sample. In any of the embodiments herein, the biological sample can be selected from the group consisting of: a blood sample, a biopsy sample, a saliva sample, a cerebrospinal fluid sample, or a urine sample. In any of the embodiments herein, the biological sample may comprise peripheral blood leukocytes. In any of the embodiments herein, the biological sample may comprise peripheral blood mononuclear cells. In any of the embodiments herein, the biological sample can be a bronchoalveolar lavage sample. In some of the embodiments herein, the biological sample is circulating nucleic acids or cell-free DNA or cell-free RNA. In any of the embodiments herein, the solid organ allograft can be one or more selected from the group consisting of: heart, lung, large intestine, small intestine, liver, kidney, pancreas, stomach, and bladder. In any of the embodiments herein, the step of detecting may comprise assaying the sample for an expression product of the at least ten genes. In any of the embodiments herein, the expression product can be a nucleic acid transcript. In any of the embodiments herein, the expression product can be a protein. In any of the embodiments herein, the step of detecting may comprise assaying the expression of the at least ten genes by hybridizing nucleic acids to oligonucleotide probes, by RT-PCR or by direct mRNA capture. In any of the embodiments herein, the step of detecting may comprise assaying the expression of the at least ten genes on one or more of: an array, a bead, and a nanoparticle. In some of the embodiments herein, the subject has an acute rejection score of Grade 0, Grade 1A, Grade 1B, Grade 2, Grade 3A, Grade 3B, or Grade 4. In any of the embodiments herein, the comparing step can identify a subject who has received a solid organ allograft for treatment of an acute rejection response with equal to or greater than 70% sensitivity. In any of the embodiments herein, the comparing step can identify a subject who has received a solid organ allograft for treatment of an acute rejection response with equal to or greater than 70% specificity. In any of the embodiments herein, the comparing step can identify a subject who has received a solid organ allograft for treatment of an acute rejection response with equal to or greater than 70% positive predictive value (ppv). In any of the embodiments herein, the comparing step can identify a subject who has received a solid organ allograft for treatment of an acute rejection response with equal to or greater than 70% negative predictive value (npv).

In yet another aspect, the invention provides methods for treating an acute rejection (AR) response in a subject who has received a solid organ allograft, the method comprising: a) detecting a gene expression level of at least ten genes in a sample from the subject, wherein the at least ten genes comprise CFLAR, DUSP1, IFNGR1, ITGAX, MAPK9, NAMPT, NKTR, PSEN1, RNF130, and RYBP; b) comparing the gene expression level to a reference expression level of the at least ten genes; c) determining the subject has an acute rejection response based upon a statistical difference or a statistical similarity between the gene expression level and the reference expression level of at least five genes selected from the group consisting of CFLAR, DUSP1, IFNGR1, ITGAX, MAPK9, NAMPT, NKTR, PSEN1, RNF130, and RYBP; and d) administering a therapeutically effective amount of one or more of a therapeutic agent to treat the acute rejection response. In some of the embodiments herein, the reference expression level is obtained from a control sample from at least one subject with an acute rejection response to a solid organ allograft. In some of the embodiments herein, the statistical similarity between the gene expression level and the reference expression level for the at least five genes determines the subject has an acute rejection response. In some of the embodiments herein, the reference expression level is obtained from a control sample from at least one subject without an acute rejection response to a solid organ allograft. In some of the embodiments herein, the statistical difference between the gene expression level and the reference expression level for the at least five genes determines the subject has an acute rejection response. In any of the embodiments herein, the sample can be a biological sample. In any of the embodiments herein, the biological sample can be selected from the group consisting of: a blood sample, a biopsy sample, a saliva sample, a cerebrospinal fluid sample, or a urine sample. In any of the embodiments herein, the biological sample can comprise peripheral blood leukocytes. In any of the embodiments herein, the biological sample can comprise peripheral blood mononuclear cells. In any of the embodiments herein, the biological sample can be a bronchoalveolar lavage sample. In some of the embodiments herein, the biological sample is circulating nucleic acids or cell-free DNA or cell-free RNA. In any of the embodiments herein, the solid organ allograft can be one or more selected from the group consisting of: heart, lung, large intestine, small intestine, liver, kidney, pancreas, stomach, and bladder. In any of the embodiments herein, the step of detecting may comprise assaying the sample for an expression product of the at least ten genes. In any of the embodiments herein, the expression product can be a nucleic acid transcript. In any of the embodiments herein, the expression product can be a protein. In any of the embodiments herein, the step of detecting may comprise assaying the expression of the at least ten genes by hybridizing nucleic acids to oligonucleotide probes, by RT-PCR or by direct mRNA capture. In any of the embodiments herein, the step of detecting may comprise assaying the expression of the at least ten genes on one or more of: an array, a bead, and a nanoparticle. In some of the embodiments herein, the subject has an acute rejection score of Grade 0, Grade 1A, Grade 1B, Grade 2, Grade 3A, Grade 3B, or Grade 4. In any of the embodiments herein, the comparing step can aid in determining the subject has an acute rejection response with equal to or greater than 70% sensitivity. In any of the embodiments herein, the comparing step aids in determining the subject has an acute rejection response with equal to or greater than 70% specificity. In any of the embodiments herein, the comparing step can aid in determining the subject has an acute rejection response with equal to or greater than 70% positive predictive value (ppv). In any of the embodiments herein, the comparing step can aid in determining the subject has an acute rejection response with equal to or greater than 70% negative predictive value (npv).

In yet another aspect, the invention provides a method of treatment of an acute rejection in a subject who has received a solid organ allograft, comprising ordering a test comprising: a) detecting a gene expression level for at least ten genes from a sample described herein, wherein the at least ten genes comprise CFLAR, DUSP1, IFNGR1, ITGAX, MAPK9, NAMPT, NKTR, PSEN1, RNF130, and RYBP; and b) comparing the gene expression level to a reference expression level obtained from a control sample, wherein the control sample is: (i) from at least one subject with an acute rejection response to a solid organ allograft, or (ii) from at least one subject without an acute rejection response to a solid organ allograft, wherein a statistical similarity for at least five genes selected from the group consisting of CFLAR, DUSP1, IFNGR1, ITGAX, MAPK9, NAMPT, NKTR, PSEN1, RNF130, and RYBP between the sample and the control sample of (i) is indicative of an acute rejection response in a subject and the treatment therapy (e.g., immunosuppressive regimen) is increased or wherein detection of a statistical similarity for at least five genes selected from the group consisting of CFLAR, DUSP1, IFNGR1, ITGAX, MAPK9, NAMPT, NKTR, PSEN1, RNF130, and RYBP between the sample and the control sample of (ii) indicates an absence of an acute rejection response in the subject and the treatment therapy (e.g., immunosuppressive regimen) is either decreased or maintained. In some of the embodiments herein, the reference expression level is obtained from a control sample from at least one subject with an acute rejection response to a solid organ allograft. In some of the embodiments herein, the statistical similarity between the gene expression level and the reference expression level for the at least five genes determines the subject has an acute rejection response. In some of the embodiments herein, the reference expression level is obtained from a control sample from at least one subject without an acute rejection response to a solid organ allograft. In some of the embodiments herein, the statistical difference between the gene expression level and the reference expression level for the at least five genes determines the subject has an acute rejection response. In any of the embodiments herein, the sample can be a biological sample. In any of the embodiments herein, the biological sample can be selected from the group consisting of: a blood sample, a biopsy sample, a saliva sample, a cerebrospinal fluid sample, or a urine sample. In any of the embodiments herein, the biological sample can comprise peripheral blood leukocytes. In any of the embodiments herein, the biological sample can comprise peripheral blood mononuclear cells. In any of the embodiments herein, the biological sample can be a bronchoalveolar lavage sample. In some of the embodiments herein, the biological sample is circulating nucleic acids or cell-free DNA or cell-free RNA. In any of the embodiments herein, the solid organ allograft can be one or more selected from the group consisting of: heart, lung, large intestine, small intestine, liver, kidney, pancreas, stomach, and bladder. In any of the embodiments herein, the step of detecting may comprise assaying the sample for an expression product of the at least ten genes. In any of the embodiments herein, the expression product can be a nucleic acid transcript. In any of the embodiments herein, the expression product can be a protein. In any of the embodiments herein, the step of detecting may comprise assaying the expression of the at least ten genes by hybridizing nucleic acids to oligonucleotide probes, by RT-PCR or by direct mRNA capture. In any of the embodiments herein, the step of detecting may comprise assaying the expression of the at least ten genes on one or more of: an array, a bead, and a nanoparticle. In some of the embodiments herein, the subject has an acute rejection score of Grade 0, Grade 1A, Grade 1B, Grade 2, Grade 3A, Grade 3B, or Grade 4. In any of the embodiments herein, the comparing step can aid in determining the subject has an acute rejection response with equal to or greater than 70% sensitivity. In any of the embodiments herein, the comparing step aids in determining the subject has an acute rejection response with equal to or greater than 70% specificity. In any of the embodiments herein, the comparing step can aid in determining the subject has an acute rejection response with equal to or greater than 70% positive predictive value (ppv). In any of the embodiments herein, the comparing step can aid in determining the subject has an acute rejection response with equal to or greater than 70% negative predictive value (npv).

In another aspect, the invention provides for methods for preparing a gene expression profile indicative of an acute rejection response to a solid organ allograft, the method comprising: a) obtaining a gene expression product from a sample of at least one subject who has received a solid organ allograft and has an acute rejection response; b) detecting the expression of at least ten genes, wherein the at least ten genes comprise CFLAR, DUSP1, IFNGR1, ITGAX, MAPK9, NAMPT, NKTR, PSEN1, RNF130, and RYBP; and c) determining the expression level for at least five genes selected from the group consisting of CFLAR, DUSP1, IFNGR1, ITGAX, MAPK9, NAMPT, NKTR, PSEN1, RNF130, and RYBP, thereby preparing the gene expression profile indicative of an acute rejection response. In another aspect, the invention provides for methods for preparing a gene expression profile indicative of an absence of an acute rejection response to a solid organ allograft, the method comprising: a) obtaining a gene expression product from a sample of at least one subject who has received a solid organ allograft and does not have an acute rejection response; b) detecting the expression of at least ten genes, wherein the at least ten genes comprise CFLAR, DUSP1, IFNGR1, ITGAX, MAPK9, NAMPT, NKTR, PSEN1, RNF130, and RYBP; and c) determining the expression level for at least five genes selected from the group consisting of CFLAR, DUSP1, IFNGR1, ITGAX, MAPK9, NAMPT, NKTR, PSEN1, RNF130, and RYBP, thereby preparing the gene expression profile indicative of the absence of an acute rejection response. In any of the embodiments herein, the sample can be a biological sample. In any of the embodiments herein, the biological sample can be selected from the group consisting of: a blood sample, a biopsy sample, a saliva sample, a cerebrospinal fluid sample, or a urine sample. In any of the embodiments herein, the biological sample may comprise peripheral blood leukocytes. In any of the embodiments herein, the biological sample may comprise peripheral blood mononuclear cells. In any of the embodiments herein, the biological sample can be a bronchoalveolar lavage sample. In some of the embodiments herein, the biological sample is circulating nucleic acids or cell-free DNA or cell-free RNA. In any of the embodiments herein, the solid organ allograft can be one or more selected from the group consisting of: heart, lung, large intestine, small intestine, liver, kidney, pancreas, stomach, and bladder. In any of the embodiments herein, the step of detecting may comprise assaying the sample for an expression product of the at least ten genes. In any of the embodiments herein, the expression product can be a nucleic acid transcript. In any of the embodiments herein, the expression product can be a protein. In any of the embodiments herein, the step of detecting may comprise assaying the expression of the at least ten genes by hybridizing nucleic acids to oligonucleotide probes, by RT-PCR or by direct mRNA capture. In any of the embodiments herein, the step of detecting may comprise assaying the expression of the at least ten genes on one or more of: an array, a bead, and a nanoparticle. In some of the embodiments herein, the subject has an acute rejection score of Grade 0, Grade 1A, Grade 1B, Grade 2, Grade 3A, Grade 3B, or Grade 4.

In still another aspect, the invention provides methods for analysis of gene expression data obtained from a subject who has received a solid organ allograft for determination of an acute rejection response, the method comprising: a) detecting the expression level for at least ten genes in a sample from the subject, wherein the at least ten genes comprise CFLAR, DUSP1, IFNGR1, ITGAX, MAPK9, NAMPT, NKTR, PSEN1, RNF130, and RYBP, thereby obtaining gene expression data from the subject; b) comparing the gene expression data to a gene expression profile prepared by method described herein; and c) determining a statistical difference or a statistical similarity between the gene expression data and the gene expression profile of at least five genes selected from the group consisting of CFLAR, DUSP1, IFNGR1, ITGAX, MAPK9, NAMPT, NKTR, PSEN1, RNF130, and RYBP. In some of the embodiments herein, the statistical similarity between the gene expression data and the gene expression profile prepared by a method described herein for the at least five genes determines the subject will have an acute response. In some of the embodiments herein, the statistical difference between the gene expression data and the gene expression profile prepared by a method described herein for the at least five genes determines the subject will not have an acute response. In some of the embodiments herein, the statistical similarity between the gene expression data and the gene expression profile prepared by a method described herein for the at least five genes determines the subject will not have an acute response. In some of the embodiments herein, the statistical difference between the gene expression data and the gene expression profile prepared by a method described herein for the at least five genes determines the subject will have an acute response. In any of the embodiments herein, the sample can be a biological sample. In any of the embodiments herein, the biological sample can be selected from the group consisting of: a blood sample, a biopsy sample, a saliva sample, a cerebrospinal fluid sample, or a urine sample. In any of the embodiments herein, the biological sample may comprise peripheral blood leukocytes. In any of the embodiments herein, the biological sample may comprise peripheral blood mononuclear cells. In any of the embodiments herein, the biological sample can be a bronchoalveolar lavage sample. In some of the embodiments herein, the biological sample is circulating nucleic acids or cell-free DNA or cell-free RNA. In any of the embodiments herein, the solid organ allograft can be one or more selected from the group consisting of: heart, lung, large intestine, small intestine, liver, kidney, pancreas, stomach, and bladder. In any of the embodiments herein, the step of detecting may comprise assaying the sample for an expression product of the at least ten genes. In any of the embodiments herein, the expression product can be a nucleic acid transcript. In any of the embodiments herein, the expression product can be a protein. In any of the embodiments herein, the step of detecting may comprise assaying the expression of the at least ten genes by hybridizing nucleic acids to oligonucleotide probes, by RT-PCR or by direct mRNA capture. In any of the embodiments herein, the step of detecting may comprise assaying the expression of the at least ten genes on one or more of: an array, a bead, and a nanoparticle. In some of the embodiments herein, the subject has an acute rejection score of Grade 0, Grade 1A, Grade 1B, Grade 2, Grade 3A, Grade 3B, or Grade 4. In some of the embodiments herein, the comparing step aids in the analysis of gene expression data for determination of acute rejection with equal to or greater than 70% sensitivity. In some of the embodiments herein, the comparing step aids in the analysis of gene expression data for determination of acute rejection with equal to or greater than 70% specificity. In some of the embodiments herein, the comparing step aids in the analysis of gene expression data for determination of acute rejection with equal to or greater than 70% positive predictive value (ppv). In some of the embodiments herein, the comparing step aids in the analysis of gene expression data for determination of acute rejection with equal to or greater than 70% negative predictive value (npv). In some of the embodiments herein, the expression level of the at least five genes is employed to predict the likelihood of an acute rejection response within 1 to 6 months of obtaining the sample.

In another aspect, the invention provides for systems for assessing an acute rejection response in a subject who has received a solid organ allograft, the system comprising: a) a gene expression evaluation element for evaluating the expression level of at least ten genes in a sample from the subject to obtain gene expression data, wherein the at least ten genes comprise CFLAR, DUSP1, IFNGR1, ITGAX, MAPK9, NAMPT, NKTR, PSEN1, RNF130, and RYBP; b) a phenotype determination element, wherein the phenotype determination element is one or more of (i) a gene expression profile indicative of an acute rejection response or (ii) a gene expression profile expression profile indicative of an absence of an acute rejection response; and c) a comparison element for comparing the gene expression data to the gene expression profile of (i) and/or (ii), wherein the comparison element compares the expression of at least five genes selected from the group consisting of CFLAR, DUSP1, IFNGR1, ITGAX, MAPK9, NAMPT, NKTR, PSEN1, RNF130, and RYBP. In any of the embodiments herein, the gene expression evaluation element may comprise one or more of: a microarray chip, an array, a bead, and a nanoparticle. In any of the embodiments herein, the gene expression evaluation element may comprise at least one reagent for assaying the sample for an expression product of the at least ten genes. In any of the embodiments herein, the expression product can be a nucleic acid transcript. In any of the embodiments herein, the expression product can be a protein. In any of the embodiments herein, the at least one reagent can be an oligonucleotide of predetermined sequence that is specific for RNA encoded by the at least ten genes. In any of the embodiments herein, the at least one reagent can be an oligonucleotide of predetermined sequence that is specific for DNA complementary to RNA encoded by the at least 10 genes. In any of the embodiments herein, the at least one reagent can be an antibody specific for a gene expression product of the at least 10 genes. In any of the embodiments herein, the phenotype determination element may be computer-generated. In any of the embodiments herein, comparison of said gene expression data to said gene expression profile can be performed by a computer or an individual. In some of the embodiments herein, a statistical similarity between the gene expression data and the gene expression profile of (i) for the at least five genes predicts the subject will have an acute rejection response. In some of the embodiments herein, a statistical difference between the gene expression data and the gene expression profile of (i) for the at least five genes predicts the subject will not have an acute rejection response. In some of the embodiments herein, a statistical similarity between the gene expression data and the gene expression profile of (ii) for the at least five genes predicts the subject will not have an acute rejection response. In some of the embodiments herein, a statistical difference between the gene expression data and the gene expression profile of (ii) for the at least five genes predicts the subject will have an acute rejection response. In any of the embodiments herein, the sample can be a biological sample. In any of the embodiments herein, the biological sample can be selected from the group consisting of: a blood sample, a biopsy sample, a saliva sample, a cerebrospinal fluid sample, or a urine sample. In any of the embodiments herein, the biological sample may comprise peripheral blood leukocytes. In any of the embodiments herein, the biological sample may comprise peripheral blood mononuclear cells. In any of the embodiments herein, the biological sample can be a bronchoalveolar lavage sample. In some of the embodiments herein, the biological sample is circulating nucleic acids or cell-free DNA or cell-free RNA. In any of the embodiments herein, the solid organ allograft can be one or more selected from the group consisting of: heart, lung, large intestine, small intestine, liver, kidney, pancreas, stomach, and bladder. In some of the embodiments herein, the subject has a cardiac acute rejection score of Grade 0, Grade 1A, Grade 1B, Grade 2, Grade 3A, Grade 3B, or Grade 4. In some of the embodiments herein, comparison of the gene expression data and the gene expression profile assesses an acute rejection response with equal to or greater than 70% sensitivity. In some of the embodiments herein, comparison of the gene expression data and the gene expression profile assesses an acute rejection response with equal to or greater than 70% specificity. In some of the embodiments herein, comparison of the gene expression data and the gene expression profile assesses an acute rejection response with equal to or greater than 70% positive predictive value (ppv). In some of the embodiments herein, comparison of the gene expression data and the gene expression profile assesses an acute rejection response with equal to or greater than 70% negative predictive value (npv). In some of the embodiments herein, the assessment of an acute rejection response in the subject predicts the likelihood of an acute rejection response within 1 to 6 months of obtaining the sample.

In another aspect, the invention provides for kits for assessing an acute rejection response in a subject who has received a solid organ allograft, the kit comprising: a) a gene expression evaluation element for evaluating the level of at least ten genes in a sample from the subject to obtain gene expression data, wherein the at least ten genes comprise CFLAR, DUSP1, IFNGR1, ITGAX, MAPK9, NAMPT, NKTR, PSEN1, RNF130, and RYBP; b) a phenotype determination element, wherein the phenotype determination element is one or more of (i) a gene expression profile indicative of an acute rejection response or (ii) a gene expression profile expression profile indicative of an absence of an acute rejection response; c) a comparison element for comparing the gene expression data to the gene expression profile of (i) and/or (ii), wherein the comparison element compares the expression of at least five genes selected from the group consisting of CFLAR, DUSP1, IFNGR1, ITGAX, MAPK9, NAMPT, NKTR, PSEN1, RNF130, and RYBP; and d) a set of instructions for assessing acute rejection response in a subject who has received a solid organ allograft. In any of the embodiments herein, the gene expression evaluation element may comprise one or more of: a microarray chip, an array, a bead, and a nanoparticle. In any of the embodiments herein, the gene expression evaluation element may comprise at least one reagent for assaying the sample for an expression product of the at least ten genes. In any of the embodiments herein, the expression product can be a nucleic acid transcript. In any of the embodiments herein, the expression product can be a protein. In any of the embodiments herein, the at least one reagent can be an oligonucleotide of predetermined sequence that is specific for RNA encoded by the at least ten genes. In any of the embodiments herein, the at least one reagent can be an oligonucleotide of predetermined sequence that is specific for DNA complementary to RNA encoded by the at least 10 genes. In any of the embodiments herein, the at least one reagent can be an antibody specific for a gene expression product of the at least 10 genes. In some of the embodiments herein, a statistical similarity between the gene expression data and the gene expression profile of (i) for the at least five genes predicts the subject will have an acute rejection response. In some of the embodiments herein, a statistical difference between the gene expression data and the gene expression profile of (i) for the at least five genes predicts the subject will not have an acute rejection response. In some of the embodiments herein, a statistical similarity between the gene expression data and the gene expression profile of (ii) for the at least five genes predicts the subject will not have an acute rejection response. In some of the embodiments herein, a statistical difference between the gene expression data and the gene expression profile of (ii) for the at least five genes predicts the subject will have an acute rejection response. In any of the embodiments herein, the sample can be a biological sample. In any of the embodiments herein, the biological sample can be selected from the group consisting of: a blood sample, a biopsy sample, a saliva sample, a cerebrospinal fluid sample, or a urine sample. In any of the embodiments herein, the biological sample may comprise peripheral blood leukocytes. In any of the embodiments herein, the biological sample may comprise peripheral blood mononuclear cells. In any of the embodiments herein, the biological sample can be a bronchoalveolar lavage sample. In some of the embodiments herein, the biological sample is circulating nucleic acids or cell-free DNA or cell-free RNA. In any of the embodiments herein, the solid organ allograft can be one or more selected from the group consisting of: heart, lung, large intestine, small intestine, liver, kidney, pancreas, stomach, and bladder. In some of the embodiments herein, the subject has a cardiac acute rejection score of Grade 0, Grade 1A, Grade 1B, Grade 2, Grade 3A, Grade 3B, or Grade 4. In some of the embodiments herein, comparison of the gene expression data and the gene expression profile assesses an acute rejection response with equal to or greater than 70% sensitivity. In some of the embodiments herein, comparison of the gene expression data and the gene expression profile assesses an acute rejection response with equal to or greater than 70% specificity. In some of the embodiments herein, comparison of the gene expression data and the gene expression profile assesses an acute rejection response with equal to or greater than 70% positive predictive value (ppv). In some of the embodiments herein, comparison of the gene expression data and the gene expression profile assesses an acute rejection response with equal to or greater than 70% negative predictive value (npv). In some of the embodiments herein, the assessment of an acute rejection response in the subject predicts the likelihood of an acute rejection response within 1 to 6 months of obtaining the sample.

### BRIEF DESCRIPTION OF THE DRAWINGS

FIG. 1 shows the study schema for development and prediction of a peripheral blood 10-gene panel for solid organ transplant rejection in pediatric and adult age study groups. A) Diagram of the process of microarray discovery and Q-PCR validation of a 10-gene panel in 489 peripheral blood samples from pediatric and young adult renal transplant recipients, with validation of the gene biomarker panel in a prospective, randomized, multicenter trial (AUC=0.937). B) Diagram of the testing of the 10 genes by Q-PCR in 141 peripheral blood samples from adult cardiac transplant recipients. A minimal logistic regression model of 5 genes was used for independent prediction for AR diagnosis in 86 samples and AR prediction prior to biopsy diagnosis.

FIG. 2 shows the histogram of the accuracy distribution for the test set prediction using 1000-time random samplings.

FIG. 3 shows the predicted probability of a sample having a non-invasive diagnosis of AR, based on the logistic regression score on the 5-gene model shown on the Y Axis (score range 0-100%). Samples with a score >37% from this model were classified as AR and samples with a score <37% from this model were classified as non-AR. The score is shown on all 141 samples, inclusive of the training (n=32; 11 Grade 3 AR, 21 STA) and the test set samples (12 CMV, 19 STA, 31 AR-Grade 1a, 22 AR-Grade 1b, 2 AR Grade 2). The clinical sample phenotype was based on the matched biopsy histology read. The misclassified samples from the histology read and the blood gene-model read are marked by asterisks.

FIG. 4 shows the individual and group predicted probabilities for all 66 AR samples. The blood-gene model classified all AR-Grade 1b correctly (a significant finding with p=0.01, for classification of other AR grades).

FIG. 5 shows the predicted probabilities for AR for all Stable samples without any evidence of acute rejection (STA), with sampling times at different times post-transplantation.

FIG. 6 shows the predicted probabilities for AR for all 55 untreated AR samples (AR-Grades≦2), where no treatment intensification was given for the diagnosis of AR. Serial samples from these patients were collected within 1-6 months prior (n=11), or within 1-6 months after (n=12), these AR episodes. The gene-model predicts AR prior to biopsy diagnosis.

FIG. 7 shows the chromosomal copy number in patient samples at different time points post-transplantation. Increases in donor derived cell-free DNA was detected months before actual organ graft injury and distinct increases in donor derived cell-free DNA was observed following different types of injury corresponding to cytomegalovirus (CMV) infection, acute rejection, or chronic injury.

### DETAILED DESCRIPTION

### I. Definitions

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

“Acute rejection” or “AR” or “acute allograft rejection” or “transplant rejection” is the rejection by the immune system of a tissue transplant recipient when the transplanted tissue is immunologically foreign. Acute rejection is characterized by infiltration of the transplanted tissue by immune cells of the recipient, which carry out their effector function and destroy the transplanted tissue. The onset of acute rejection is rapid and generally occurs in humans within 6-12 months after transplant surgery. Generally, acute rejection can be inhibited or suppressed with immunosuppressive drugs such as rapamycin, cyclosporine A, anti-CD40L monoclonal antibodies, and the like.

The term “solid organ allograft” is a solid organ transplant from one individual to another individual.

As used herein, “gene” refers to a nucleic acid comprising an open reading frame encoding a polypeptide, including exon and (optionally) intron sequences. The term “intron” refers to a DNA sequence present in a given gene that is not translated into protein and is generally found between exons in a DNA molecule. In addition, a gene may optionally include its natural promoter (i.e., the promoter with which the exon and introns of the gene are operably linked in a non-recombinant cell), and associated regulatory sequences, and may or may not include sequences upstream of the AUG start site, untranslated leader sequences, signal sequences, downstream untranslated sequences, transcriptional start and stop sequences, polyadenylation signals, translational start and stop sequences, ribosome binding sites, and the like.

The term “reference” refers to a known value or set of known values against which an observed value may be compared. In one embodiment, the reference is the value (or level) of gene expression of a gene indicative of an absence or presence of an acute rejection response.

As used herein, “reference expression level” or “gene expression profile” refers to a reference standard or a predetermined set of values representing the expression levels of the genes of interest described herein that are previously generated using a control or reference sample. In one embodiment, the reference expression level or gene expression profile is a reference standard created for AR samples for each differentially expressed gene. In another embodiment, the reference expression level or gene expression profile is a reference standard created for non-AR samples for each differentially expressed gene.

As used herein, “gene expression data” refers to the expression of a gene or set of genes through the detection of a nucleic acid or protein from a sample. In some embodiments, the term “gene expression data” refers to gene expression data for a set of genes that is obtained from a subject or subjects who have had an organ transplant, wherein the gene expression data is compared to a “reference expression level” or “gene expression profile” to assess or determine if a subject has an allograft rejection.

A “subject” can be a “patient” or an “individual.” A “patient” refers to an “individual” or “subject” who is under the care of a treating physician. The patient can be male or female of about 1 year of age to greater than about 100 years of age, including all years in the specified age range. In one embodiment, the patient has received a solid organ transplant. In another embodiment, the patient has received a solid organ transplant and is underdoing organ rejection. In yet another embodiment, the patient has received a solid organ transplant and is undergoing acute rejection.

A “patient sub-population,” and grammatical variations thereof, as used herein, refers to a patient subset characterized as having one or more distinctive measurable and/or identifiable characteristics that distinguishes the patient subset from others in the broader disease category to which it belongs.

The term “sample,” as used herein, refers to a composition that is obtained or derived from a subject that contains genetic information. In one embodiment, the sample is blood. In another embodiment, the sample is peripheral blood leukocytes. In another embodiment, the sample is peripheral blood mononuclear cells. In another embodiment, the biological sample is circulating nucleic acids or cell-free DNA or cell-free RNA.

As used herein, “microarray” or “array” refers to an arrangement of a collection of nucleotide sequences in a centralized location. Arrays can be on a solid substrate, such as a surface composed of glass, plastic, or silicon. The nucleotide sequences can be DNA, RNA, or any permutation thereof. The nucleotide sequences can also be partial sequences from a gene, primers, whole gene sequences, non-coding sequences, coding sequences, published sequences, known sequences, or novel sequences.

“Predicting” and “prediction” as used herein does not mean that the outcome is occurring with 100% certainty. Instead, it is intended to mean that the outcome is more likely occurring than not. Acts taken to “predict” or “make a prediction” can include the determination of the likelihood that an outcome is more likely occurring than not. Assessment of multiple factors described herein can be used to make such a determination or prediction.

The term “diagnosis” is used herein to refer to the identification or classification of a molecular or pathological state, disease, or condition. For example, “diagnosis” may refer to identification of an organ rejection. “Diagnosis” may also refer to the classification of a particular sub-type of organ rejection, such as acute rejection.

By “compare” or “comparing” is meant correlating, in any way, the results of a first analysis with the results of a second and/or third analysis. For example, one may use the results of a first analysis to classify the result as more similar to a second result than to a third result. With respect to the embodiment of AR assessment of biological samples from an individual, one may use the results to determine whether the individual is undergoing an AR response.

The term “determining” can refer to any form of measurement, and include both quantitative and qualitative measurements. For example, “determining” may be relative or absolute.

The terms “assessing or “assessment” encompasses the prediction, diagnosis, monitoring, detection, or identification of an acute rejection response in a subject.

As used herein, “treatment” refers to clinical intervention in an attempt to alter the natural course of the individual being treated. Desirable effects of treatment include preventing the occurrence or recurrence of a disease or a condition or symptom thereof, alleviating a condition or symptom of the disease, diminishing any direct or indirect pathological consequences of the disease, decreasing the rate of disease progression, ameliorating or palliating the disease state, and achieving improved prognosis.

Reference to “about” a value or parameter herein includes (and describes) embodiments that are directed to that value or parameter per se. For example, description referring to “about X” includes description of “X”. The term “about” is used to provide flexibility to a numerical range endpoint by providing that a given value may be “a little above” or “a little below” the endpoint without affecting the desired result. Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited.

It is understood that aspects and embodiments of the invention described herein include “consisting of” and/or “consisting essentially of” aspects and embodiments.

As used in the specification and the appended claims, the singular forms “a,” “an,” and “the” include plural referents unless the context clearly indicates otherwise.

### II. General Techniques

Unless defined otherwise, technical and scientific terms used herein have the same meaning as commonly understood by one of ordinary skill in the art to which this invention belongs.

The practice of the present invention will employ, unless otherwise indicated, conventional techniques of protein biology, protein chemistry, molecular biology (including recombinant techniques), microbiology, cell biology, biochemistry, and immunology, which are within the skill of the art. Such techniques are explained fully in the literature, such as “Molecular Cloning: A Laboratory Manual”, second edition (Sambrook et al., 1989); “Current Protocols in Molecular Biology” (Ausubel et al., eds., 1987, periodic updates); “PCR: The Polymerase Chain Reaction”, (Mullis et al., eds., 1994); and Singleton et al., Dictionary of Microbiology and Molecular Biology, 2nd ed., J. Wiley & Sons (New York, N.Y. 1994).

### III. Collection and Processing of Biological Samples

In some aspects of the methods, compositions, systems, or kits described herein, a sample from a subject e.g., a biological sample), is assayed to monitor for an AR response to a graft (e.g., a solid organ allograft). In some embodiments, the first step of a method described herein is to obtain a suitable sample from a subject of interest, i.e., a subject who has received at least one graft (e.g., a solid organ allograft). In some embodiments, a subject of interest (e.g., a subject who has received a solid organ allograft) is a mammal. Non-limiting examples of mammals include those of the orders carnivore (e.g., dogs and cats), rodentia (e.g., mice, guinea pigs, hamsters, and rats), lagomorpha (e.g., rabbits) and non-human primates (e.g., chimpanzees, apes, prosimians, and monkeys). In certain embodiments, the subject of interest is a human. A subject of interest includes one who is to be tested, or has been tested for assessment (e.g., prediction, diagnosis, identification, etc.) of allograft rejection. The subject may have been previously assessed or diagnosed using other methods, such as those described herein or those in current clinical practice, or maybe selected as part of a general population (a control subject).

In some embodiments, the sample obtained from the subject is a biological sample. The sample obtained from the subject can derived from any suitable source. Suitable sources include, but are not limited to, cerebro-spinal fluid (CSF), urine, saliva, tears, lymph fluid, tissue derived samples (e.g., homogenates (such as biopsy samples of the transplanted tissue or organ)), and blood or derivatives thereof. In some embodiments the suitable source is a biopsy sample of a transplanted heart, kidney, lung, liver, pancreas, pancreatic islets, brain tissue, stomach, large intestine, small intestine, cornea, skin, trachea, bone, bone marrow, muscle, bladder or parts thereof. In some embodiments, the sample is a blood sample or blood-derived sample. In some embodiments, the blood-derived sample is derived from whole blood or a fraction thereof, e.g., serum, plasma, cellular fraction, etc. In some embodiments, the sample is derived from blood cells harvested from whole blood. In some embodiments, the sample is peripheral blood mononuclear cells/lymphocytes (PBMCs/PBLs). In some embodiments, the sample is peripheral blood leukocytes. In some aspects, the sample comprises an early blood stem cell (e.g., a hematopoeitic stem cell or hemangioblast), a myeloid progenitor or lymphoid progenitor, mast cells, myeloblasts, basophils, neutrophils, eosinophils, monocytes, macrophages, large granular lymphocytes (e.g., natural killer cells), T lymphocytes, B lymphocytes, or plasma cells. Any convenient protocol for obtaining such samples may be employed, where suitable protocols are well known in the art (e.g., density gradient fractionation of a whole blood sample) and a representative protocol is reported in the Experimental Section, below.

In some embodiments, samples are derived from an animal (e.g., a human) comprising different sample sources comprising biological fluids, solid tissue samples, or semi-solid tissues that can include but is not limited to, for example whole blood, sweat, tears, saliva, ear flow, sputum, lymph, bone marrow suspension, lymph, urine, saliva, semen, vaginal flow, cerebrospinal fluid, brain fluid, ascites, milk, secretions of the respiratory, intestinal or genitourinary tracts fluid, a lavage of a tissue or organ (e.g. lung) or tissue, which has been removed from organs (e.g., a tissue biopsy), such as breast, lung, intestine, skin, cervix, prostate, pancreas, heart, liver and stomach.

In some embodiments, methods of the invention provide for the non-invasive diagnostic testing of organ transplant patients by obtaining circulating nucleic acids or cell-free DNA or cell-free RNA from any of the sample sources described herein. In one aspect, circulating nucleic acids or cell-free DNA or cell-free RNA is obtained from a biological fluid. In one aspect, circulating nucleic acids or cell-free DNA or cell-free RNA is obtained from whole blood. In another aspect, circulating nucleic acids or cell-free DNA or cell-free RNA is quantitated for the diagnosis, prognosis, detection and/or treatment of a transplant or solid organ allograft status or outcome (U.S. Pat. No. 8,703,652 is incorporated by reference solely for its description thereof).

In some embodiments, when obtaining a sample from a subject (e.g., blood sample), the amount can vary depending upon subject size and the condition being screened. In some aspects, up to 50, 40, 30, 20, 10, 9, 8, 7, 6, 5, 4, 3, 2, or 1 mL of a sample is obtained. In some aspects, 1-50, 2-40, 3-30, or 4-20 mL of sample is obtained. In some aspects, more than 5, 10, 15, 20, 25, 30, 35, 40, 45, 50, 55, 60, 65, 70, 75, 80, 85, 90, 95 or 100 mL of a sample is obtained. In some aspects, less than 1 pg, 5 pg, 10 pg, 20 pg, 30 pg, 40 pg, 50 pg, 100 pg, 200 pg, 500 pg, 1 ng, 5 ng, 10 ng, 20 ng, 30 ng, 40 ng, 50 ng, 100 ng, 200 ng, 500 ng, 1 ug, 5 ug, 10 ug, 20 ug, 30 ug, 40 ug, 50 ug, 100 ug, 200 ug, 500 ug or 1 mg of nucleic acids (e.g., cell-free DNA or cell-free RNA) are obtained from the sample for further genetic analysis. In some aspects, about 1-5 pg, 5-10 pg, 10-100 pg, 100 pg-1 ng, 1-5 ng, 5-10 ng, 10-100 ng, 100 ng-1 ug of nucleic acids (e.g., cell-free DNA or cell-free RNA) are obtained from the sample for further genetic analysis.

The methods described herein may be used to monitor a variety of different types of solid organ allografts, Solid organ allografts of interest include, but are not limited to: transplanted heart, kidney, lung, liver, pancreas, pancreatic islets, brain tissue, stomach, large intestine, small intestine, cornea, skin, trachea, bone, bone marrow, muscle, bladder or parts thereof. A plurality of biological samples may be collected at any one time. A biological sample or samples may be taken from a subject at any time, including before allograft transplantation, at the time of transplantation, or at any time following transplantation.

In some embodiments, the sample obtained from the subject is prepared fir evaluation by isolating RNA from the sample using methods described herein, and deriving (obtaining) complementary DNA (cDNA) from the isolated RNA by reverse transcription techniques. However, other methods can be used to obtain RNA, and these methods are known to those of skill in the art. In some embodiments, whether the subject will have an acute rejection response is determined based upon a statistical difference or a statistical similarity between the gene expression level and the reference expression level for at least five genes selected from the group consisting of CFLAR, DUSP1, IFNGR1, ITGAX, MAPK9, NAMPT, NKTR, PSEN1, RNF130, and RYBP. In some embodiments the reference expression level is obtained from a control sample from at least one subject with an acute rejection response to a solid organ allograft. In some embodiments the reference expression level is obtained from a control sample from at least one subject without an acute rejection response to a solid organ allograft. In some embodiments, the sample Obtained from the subject is prepared for evaluation by isolating proteins or fragments thereof using methods known to those of skill in the art. In some embodiments, the proteins, or fragments thereof, encoded by any of the genes that are described herein may be detected using western blot, protein arrays, or other techniques known to those of skill in the art. In some embodiments, whether the subject will have an acute rejection response is determined based upon a statistical difference or a statistical similarity between the protein level in the subject and the protein level in a reference sample for the proteins encoded by at least five genes selected from the group consisting of CFLAR, DUSP1, IFNGR1, ITGAX, MAPK9, NAMPT, NKTR, PSEN1, RNF130, and RYBP, In some embodiments the reference protein level is obtained from a control sample from at least one subject with an acute rejection response to a solid organ allograft. In some embodiments the reference protein level is obtained from a control sample from at least one subject without an acute rejection response to a solid organ allograft. In some embodiments, protein levels are detected in a post-transplant fluid sample such as blood or urine, Normalization of protein levels may be performed in much the same way as normalization of transcript levels. One or more constitutively or universally produced proteins may be detected and used for normalization.

In some embodiments, a subject of interest belongs to a patient sub-population. For example, any of the methods described herein may have use in assessing acute rejection in a subject with a cardiac allograft acute rejection score of Grade 0, Grade 1A, Grade 1B, Grade 2, Grade 3A, Grade 3B, or Grade 4. In some embodiments, a patient sub-population assessed by a method, compositions, systems or kits described herein is a patient that does not have a cardiac allograft acute rejection score of Grade 3A, Grade 3B, or Grade 4. This sub-population of patients may or may not have a cardiac allograft acute rejection score of Grade 0, Grade 1A, Grade 1B, or Grade 2. This sub-population may or may not have had a cardiac biopsy. Use of any of the methods, compositions, systems or kits described herein can non-invasively assess an acute rejection response in a sub-population of patients that possibly has a cardiac allograft acute rejection score of Grade 0, Grade 1A, Grade 1B, or Grade 2.

Also provided herein are methods for preparing a gene expression profile indicative of an acute rejection response to a solid organ allograft, the method comprising: a) obtaining a gene expression product from a sample of at least one subject who has received a solid organ allograft and has an acute rejection response; b) detecting the expression of at least ten genes, wherein the at least ten genes comprise CFLAR, DUSP1, IFNGR1, ITGAX, MAPK9, NAMPT, NKTR, PSEN1, RNF130, and RYBP; and c) determining the expression level for at least five genes selected from the group consisting of CFLAR, DUSP1, IFNGR1, ITGAX, MAPK9, NAMPT, NKTR, PSEN1, RNF130, and RYBP, thereby preparing the gene expression profile indicative of an acute rejection response. In some embodiments, also provided is a method for preparing a gene expression profile indicative of an absence of an acute rejection response to a solid organ allograft, the method comprising: a) obtaining a gene expression product from a sample of at least one subject who has received a solid organ allograft and does not have an acute rejection response; b) detecting the expression of at least ten genes, wherein the at least ten genes comprise CFLAR, DUSP1, IFNGR1, ITGAX, MAPK9, NAMPT, NKTR, PSEN1, RNF130, and RYBP; and c) determining the expression level for at least five genes selected from the group consisting of CFLAR, DUSP1, IFNGR1, ITGAX, MAPK9, NAMPT, NKTR, PSEN1, RNF130, and RYBP, thereby preparing the gene expression profile indicative of the absence of an acute rejection response. Gene expression profiles prepared by the methods described herein can find use in any of the methods described herein for assessing an acute rejection response in a subject who has received a solid organ allograft. Such gene expression profiles described herein allow for the determination of a statistical similarity and/or statistical difference to be assessed in the methods described herein with one or more of a 70% or greater sensitivity, specificity, positive predictive value (ppv) and negative predictive value (npv), or any other explicit numerical value described herein for these parameters.

Specificity

The specificity of a model can be a measure of the proportion of subjects that are actually negative for a condition which are correctly identified as being negative for the condition by the model. The specificity of a model can be equal to the number of true negatives divided by the sum of the number of true negatives and false positives. In other words, the specificity of a model can be the probability of a negative test result given that the subject is actually negative for the condition. In some embodiments of the present invention, the specificity of the methods described herein is the number of subjects without AR that were predicted by the methods described herein to not have AR divided by the total number of subjects predicted to not have AR using the methods described herein. In some embodiments, the comparing step of the methods described herein comprises assessing (e.g., predicting, diagnosing, identifying, etc.) an acute rejection response with a specificity of about 70-100%. In some embodiments, the specificity is about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, but no more than 100%. In some embodiments, the specificity is about 70-75%, 75-80%, 80-85%, 85-90%, 90-95%, or 95-100%, but no more than 100%. In some embodiments the specificity is about 70%. In some embodiments the specificity is about 90%.

Sensitivity

The sensitivity of a model can be a measure of the proportion of subjects that are actually positive for a condition which are correctly identified as being positive for the condition by the model. The sensitivity of a model can be equal to the number of true positives divided by the sum of the number of true positives and false negatives. In other words, the sensitivity of a model can be the probability of a positive test result given that the subject is actually positive for the condition. In some embodiments of the present invention, the sensitivity of the methods herein is the number of subjects with AR that were predicted by the methods described herein to have AR divided by the total number of subjects predicted to have AR using the methods described herein. In some embodiments, the comparing step of the methods described herein comprises assessing (e.g., predicting, diagnosing, identifying, etc.) an acute rejection response with a sensitivity of about 70-100%. In some embodiments, the sensitivity is about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, but no more than 100%. In some embodiments, the sensitivity is about 70-75%, 75-80%, 80-85%, 85-90%, 90-95%, or 95-100%, but no more than 100%. In some embodiments the sensitivity is about 70%. In some embodiments the sensitivity is about 87%.

Positive Predictive Value

The positive predictive value of a model can be the proportion of positive test results that are true positives. The positive predictive value can be equal to the number of true positives divided by the sum of the number of true positives and the number of false positives. A “true positive” is the event that the model makes a positive prediction, and the subject actually has the condition. A “false positive” is the event that the model makes a positive prediction, and the subject does not have the condition. In some embodiments of the present invention, the positive predictive value is the number of subjects with AR that are prediaed to have AR based on the methods described herein, divided by the total number of subjects predicted to have AR based on the methods described herein. In some embodiments, the comparing step of the methods described herein comprises assessing (e.g., predicting, diagnosing, identifying, etc.) an acute rejection response with a positive predictive value of about 70-100%. In some embodiments, the positive predictive value is about 70%, 71%, 72%, 73%, 74%, 75%, 76%, 77%, 78%, 79%, 80%, 81%, 82%, 83%, 8.4%, 85%, 86%, 87%, 88%, 89%, 90%, 91%. 92%, 93%, 94%, 95%, 96%, 97%, 98%, 99%, or 100%, but no more than 100%. In some embodiments, the positive predictive value is about 70-75%, 75-80%, 80-85%, 85-90%, 90-95%, or 95-100%, but no more than 100%. In some embodiments the positive predictive value is about 70%. In some embodiments the positive predictive value is about 94%.

Negative Predictive Value

The negative predictive value of a model can be the proportion of negative test results that are true negatives. The negative predictive value can be equal to the number of true negatives divided by the sum of the number of true negatives and the number of false negatives. A “true negative” is the event that the model makes a negative prediction, and the subject does not have the condition. A “false negative” is the event that the model makes a negative prediction, and the subject actually has the condition. In some embodiments of the present invention, the negative predictive value is the number of subjects without AR that are predicted to not have AR based on the methods described herein, divided by the total number of subjects predicted to not have AR based on the methods described herein. In some embodiments, the comparing step of the methods herein comprises assessing (e.g., predicting, diagnosing, identifying, etc.) an acute rejection response with a negative predictive value of about 70-100%, In some embodiments, the negative predictive value is about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, but no more than 100%. In some embodiments, the negative predictive value is about 70-75%, 75-80%, 80-85%, 85-90%, 90-95%, or 95-100%, but no more than 100%. In some embodiments the negative predictive value is about 70%. In some embodiments the negative predictive value is about 80%.

### IV. Methods for Assessing an Acute Rejection Response

In some aspects, provided herein is a method for aiding in the diagnosis of an acute rejection response in a subject who has received a solid organ allograft, the method comprising: a) detecting a gene expression level for at least ten genes in a sample from the subject, wherein the at least ten genes comprise CFLAR, DUSP1, IFNGR1, ITGAX, MAPK9, NAMPT, NKTR, PSEN1, RNF130, and RYBP; and b) comparing the gene expression level to a reference expression level of the at least ten genes, wherein a statistical difference or a statistical similarity between the gene expression level and the reference expression level of at least five genes selected from the group consisting of CFLAR, DUSP1, IFNGR1, ITGAX, MAPK9, NAMPT, NKTR, PSEN1, RNF130, and RYBP, thereby aiding in the diagnosis of an acute rejection response. In some embodiments, the method for aiding in the diagnosis of an acute rejection response in a subject who has received a solid organ allograft comprises: a) detecting a gene expression level for at least ten genes in a sample from the subject, wherein the at least ten genes comprise CFLAR, DUSP1, IFNGR1, ITGAX, MAPK9, NAMPT, NKTR, PSEN1, RNF130, and RYBP; and b) comparing the gene expression level to a reference expression level obtained from a control sample, wherein the control sample is: (i) from at least one subject with an acute rejection response to a solid organ allograft, or (ii) from at least one subject without an acute rejection response to a solid organ allograft, wherein a statistical similarity for at least five genes selected from the group consisting of CFLAR, DUSP1, IFNGR1, ITGAX, MAPK9, NAMPT, NKTR, PSEN1, RNF130, and RYBP between the sample and the control sample of (i) aids in the diagnosis of an acute rejection response in the subject or wherein detection of a statistical similarity for at least five genes selected from the group consisting of CFLAR, DUSP1, IFNGR1, ITGAX, MAPK9, NAMPT, NKTR, PSEN1, RNF130, and RYBP between the sample and the control sample of (ii) aids in the diagnosis of the absence of an acute rejection response in the subject. In some embodiments, the method for aiding in the diagnosis of an acute rejection response in a subject who has received a solid organ allograft comprises: a) detecting a gene expression level for at least ten genes in the sample, wherein the at least ten genes comprise CFLAR, DUSP1, IFNGR1, ITGAX, MAPK9, NAMPT, NKTR, PSEN1, RNF130, and RYBP; and b) comparing the gene expression level to a reference expression level obtained from a control sample, wherein the control sample is: (i) from at least one subject with an acute rejection response to a solid organ allograft, or (ii) from at least one subject without an acute rejection response to a solid organ allograft, wherein a statistical difference for at least five genes selected from the group consisting of CFLAR, DUSP1, IFNGR1, ITGAX, MAPK9, NAMPT, NKTR, PSEN1, RNF130, and RYBP between the sample and the control sample of (i) aids in the diagnosis of the absence of an acute rejection response in the subject or wherein detection of a statistical difference for at least five genes selected from the group consisting of CFLAR, DUSP1, IFNGR1, ITGAX, MAPK9, NAMPT, NKTR, PSEN1, RNF130, and RYBP between the sample and the control sample of (ii) aids in the diagnosis of an acute rejection response in the subject.

Non-limiting variations of a method of aiding in the diagnosis of an acute rejection response in a subject who has received a solid organ allograft are contemplated herein. In some embodiments, a method for aiding in the diagnosis of an acute rejection response in a subject who has received a solid organ allograft may comprise: a) measuring, by hybridization assay, a gene expression level for at least ten genes in a sample from the subject, wherein the at least ten genes comprise CFLAR, DUSP1, IFNGR1, ITGAX, MAPK9, NAMPT, NKTR, PSEN1, RNF130, and RYBP; b) comparing the gene expression level to a reference expression level of the at least ten genes; and c) diagnosing an acute rejection response in the subject based upon a statistical difference or a statistical similarity between the gene expression level and the reference expression level of at least five genes selected from the group consisting of CFLAR, DUSP1, IFNGR1, ITGAX, MAPK9, NAMPT, NKTR, PSEN1, RNF130, and RYBP, thereby aiding in the diagnosis of an acute rejection response in the subject. In another embodiment, a method for aiding in the diagnosis of an acute rejection response in a subject who has received a solid organ allograft may comprise: a) for each gene of a set of genes comprising CFLAR, DUSP1, IFNGR1, ITGAX, MAPK9, NAMPT, NKTR, PSEN1, RNF130, and RYBP, detecting the level of RNA encoded by the gene in a sample from the test subject using at least one oligonucleotide of predetermined sequence which is specific for RNA encoded by the gene and/or for DNA complementary to RNA encoded by the gene, thereby obtaining a gene expression level for the gene; and b) applying logistic regression analysis to the gene expression level of at least five genes selected from the group consisting of CFLAR, DUSP1, IFNGR1, ITGAX, MAPK9, NAMPT, NKTR, PSEN1, RNF130, and RYBP to classify the subject as more likely to either have acute rejection or not have acute rejection, wherein the logistic regression analysis is performed using a logistic regression model fitted to levels of RNA encoded by the genes in a sample of subjects having acute rejection, and levels of RNA encoded by the genes in a samples of subjects not having acute rejection, thereby diagnosing the test subject as more likely to either have acute rejection or not have acute rejection. In yet another embodiment, a method for aiding in the diagnosis of an acute rejection response in a subject who has received a solid organ allograft may comprise: a) contacting a sample from the subject who has received a solid organ allograft with a nucleic acid that specifically binds each of genes CFLAR, DUSP1, IFNGR1, ITGAX, MAPK9, NAMPT, NKTR, PSEN1, RNF130, and RYBP; b) detecting a gene expression level for each of the genes; and c) comparing the gene expression level to a reference expression level of genes CFLAR, DUSP1, IFNGR1, ITGAX, MAPK9, NAMPT, NKTR, PSEN1, RNF130, and RYBP, wherein a statistical difference or a statistical similarity between the gene expression level and the reference expression level of at least five genes selected from the group consisting of CFLAR, DUSP1, IFNGR1, ITGAX, MAPK9, NAMPT, NKTR, PSEN1, RNF130, and RYBP, thereby aiding in the diagnosis of an acute rejection response in the subject.

In some embodiments herein, a method for aiding in the diagnosis comprises an additional step of procuring a sample from the subject who has received a solid organ allograft. For example, a method for aiding in the diagnosis of an acute rejection response in a subject who has received a solid organ allograft may comprise: a) obtaining a sample from the subject who has received a solid organ allograft; b) detecting a gene expression level for at least ten genes in a sample from the subject, wherein the at least ten genes comprise CFLAR, DUSP1, IFNGR1, ITGAX, MAPK9, NAMPT, NKTR, PSEN1, RNF130, and RYBP; and c) comparing the gene expression level to a reference expression level of the at least ten genes, wherein a statistical difference or a statistical similarity between the gene expression level and the reference expression level of at least five genes selected from the group consisting of CFLAR, DUSP1, IFNGR1, ITGAX, MAPK9, NAMPT, NKTR, PSEN1, RNF130, and RYBP, thereby aiding in the diagnosis of an acute rejection response.

In some embodiments of the methods described herein, the methods have use in predicting an acute rejection response. In these methods, a subject is first monitored for acute rejection according to the subject methods, and then treated using a protocol determined, at least in part, on the results of the monitoring. In one embodiment, the subject is monitored for the presence or absence of acute rejection according to one of the methods described herein. The subject may then be treated using a protocol whose suitability is determined using the results of the monitoring step. For example, where the subject is predicted to have an acute rejection response within the next 1 to 6 months, immunosuppressive therapy can be modulated, e.g., increased or drugs changed, as is known in the art for the treatment/prevention of acute rejection. Likewise, where the subject is predicted to be free of current and near-term acute rejection, the immunosuppressive therapy can be reduced in order to reduce the potential for drug toxicity. In some, embodiments of the methods described herein, a subject is monitored for acute rejection following receipt of a graft or transplant. The subject may be screened once or serially following transplant receipt, e.g., weekly, monthly, bimonthly, half-yearly, yearly, etc. In some embodiments, the subject is monitored prior to the occurrence of an acute rejection episode. In other embodiments, the subject is monitored following the occurrence of an acute rejection episode.

In some embodiments of the methods described herein, the methods have use in altering or changing a treatment paradigm or regimen of a subject in need of treatment of an allograft rejection. Exemplary non-limiting immunosuppressive therapeutics or therapeutic agents useful for the treating of a subject in need thereof comprise steroids (e.g., prednisone (Deltasone), prednisolone, methyl-prednisolone (Medrol, Solumedrol)), antibodies (e.g., muromonab-CD3 (Orthoclone-OKT3), antithymocyte immune globulin (ATGAM, Thymoglobulin), daclizumab (Zenapax), basiliximab (Simulect), Rituximab, cytomegalovirus-immune globulin (Cytogam), immune globulin (Polygam)), calcineurin inhibitors (e.g., cyclosporine (Sandimmune), tacrolimus (Prograf)), antiproliferatives (e.g., mycophenolate mofetil (Cellcept), azathioprine (Imuran)), TOR inhibitors (e.g., rapamycin (Rapamune, sirolimus), everolimus (Certican)), or a combination therapy thereof.

In some embodiments, wherein a subject is identified as not having an acute allograft rejection using the methods described herein, the subject can remain on an immunosuppressive standard of care maintenance therapy comprising the administration of an antiproliferative agent (e.g., mycophenolate mofetil and/or azathioprine), a calcineurin inhibitor (e.g., cyclosporine and/or tacrolimus), steroids (e.g., prednisone, prednisolone, and/or methyl prednisolone) or a combination thereof. For example, a subject identified as not having an acute allograft rejection using the methods described herein can be placed on a maintenance therapy comprising the administration of a low dose of prednisone (e.g., about 0.1 mg·kg⁻¹·d⁻¹ to about 1 mg·kg⁻¹·d⁻¹), a low dose of cyclosporine (e.g., about 4 mg·kg⁻¹·d⁻¹ to about 8 mg·kg⁻¹·d⁻¹), and a low dose of mycophenolate (e.g., about 1-1.5 g twice daily). In another example, a subject identified as not having an acute allograft rejection using the methods described herein can be taken off of steroid therapy and placed on a maintenance therapy comprising the administration of a low dose of cyclosporine (e.g., about 4 mg·kg⁻¹·d⁻¹ to about 8 mg·kg⁻¹·d⁻¹), and a low dose of mycophenolate (e.g., about 1-1.5 g twice daily). In another example, a subject identified as not having an acute allograft rejection using the methods described herein can be removed from all immunosuppressive therapeutics described herein.

In some embodiments, wherein a subject is identified as having an acute allograft rejection using the methods described herein, the subject may be placed on a rescue therapy or increase in immunosuppressive agents comprising the administration of a high dose of a steroid (e.g., prednisone, prednisolone, and/or methyl prednisolone), a high dose of a polyclonal or monoclonal antibody (e.g., muromonab-CD3 (OKT3), antithymocyte immune globulin, daclizumab, basiliximab, cytomegalovirus-immune globulin, and/or immune globulin), a high dose of an antiproliferative agent (e.g., mycophenolate mofetil and/or azathioprine), or a combination thereof.

In some embodiments, the course of therapy wherein a subject is identified as not having an acute allograft rejection or is identified as having an acute allograft rejection using the methods described herein is dependent upon the time after transplantation and the severity of rejection, treating physician, and the transplantation center.

In some aspects, provided herein is a method of treatment of an acute rejection in a subject who has received a solid organ allograft, comprising ordering a test comprising: a) detecting a gene expression level for at least ten genes from a sample described herein, wherein the at least ten genes comprise CFLAR, DUSP1, IFNGR1, ITGAX, MAPK9, NAMPT, NKTR, PSEN1, RNF130, and RYBP; and b) comparing the gene expression level to a reference expression level obtained from a control sample, wherein the control sample is: (i) from at least one subject with an acute rejection response to a solid organ allograft, or (ii) from at least one subject without an acute rejection response to a solid organ allograft, wherein a statistical similarity for at least five genes selected from the group consisting of CFLAR, DUSP1, IFNGR1, ITGAX, MAPK9, NAMPT, NKTR, PSEN1, RNF130, and RYBP between the sample and the control sample of (i) is indicative of an acute rejection response in a subject and the treatment therapy (e.g., immunosuppressive regimen) is increased or wherein detection of a statistical similarity for at least five genes selected from the group consisting of CFLAR, DUSP1, IFNGR1, ITGAX, MAPK9, NAMPT, NKTR, PSEN1, RNF130, and RYBP between the sample and the control sample of (ii) indicates an absence of an acute rejection response in the subject and the treatment therapy (e.g., immunosuppressive regimen) is either decreased or maintained.

In some aspects, provided herein is a method for predicting the likelihood of an acute rejection response in a subject who has received a solid organ allograft, the method comprising: a) detecting a gene expression level for at least ten genes in a sample from the subject, wherein the at least ten genes comprise CFLAR, DUSP1, IFNGR1, ITGAX, MAPK9, NAMPT, NKTR, PSEN1, RNF130, and RYBP; and b) comparing the gene expression level to a reference expression level of the at least ten genes, wherein a statistical difference or a statistical similarity between the gene expression level and the reference expression level for at least five genes selected from the group consisting of CFLAR, DUSP1, IFNGR1, ITGAX, MAPK9, NAMPT, NKTR, PSEN1, RNF130, and RYBP, thereby predicting the likelihood of an acute rejection response in the subject. In some embodiments, the expression level of the at least five genes is employed to predict the likelihood of an acute rejection response within 1 to 6 months of obtaining the sample. For example, the expression level of the at least five genes can be employed to predict the likelihood of an acute rejection response within 1, 2, 3, 4, 5, and/or 6 months of procuring (e.g., obtaining) the sample. In some embodiments herein, a method for predicting the likelihood of an acute rejection response comprises an additional step of procuring a sample from the subject who has received a solid organ allograft.

In some aspects, provided herein is a method for monitoring the progression of an acute rejection response in a subject who has received a solid organ allograft, the method comprising: a) detecting a gene expression level for at least ten genes in a sample from the subject, wherein the at least ten genes comprise CFLAR, DUSP1, IFNGR1, ITGAX, MAPK9, NAMPT, NKTR, PSEN1, RNF130, and RYBP; b) comparing the gene expression level to a reference expression level of the at least ten genes; and c) determining whether the subject has an acute rejection response based upon a statistical difference or a statistical similarity between the gene expression level and the reference expression level of at least five genes selected from the group consisting of CFLAR, DUSP1, IFNGR1, ITGAX, MAPK9, NAMPT, NKTR, PSEN1, RNF130, and RYBP, thereby monitoring the progression of an acute rejection response in the subject. For example, the method for monitoring progression of an acute rejection response can comprise the steps of: a) detecting a gene expression level for at least ten genes in a first sample from the subject at a first period of time, wherein the at least ten genes comprise CFLAR, DUSP1, IFNGR1, ITGAX, MAPK9, NAMPT, NKTR, PSEN1, RNF130, and RYBP; b) detecting a gene expression level for the at least ten genes in a second sample from the subject at a second period of time; c) comparing the gene expression level in step (a) to the amount detected in step (b), wherein the acute rejection is progressing if the gene expression level of at least five genes selected from the group consisting of CFLAR, DUSP1, IFNGR1, ITGAX, MAPK9, NAMPT, NKTR, PSEN1, RNF130, and RYBP remains constant over time. In another example, the method for monitoring progression of an acute rejection response can comprise the steps of: a) detecting a gene expression level for at least ten genes in a first sample from the subject at a first period of time, wherein the at least ten genes comprise CFLAR, DUSP1, IFNGR1, ITGAX, MAPK9, NAMPT, NKTR, PSEN1, RNF130, and RYBP; b) detecting a gene expression level for the at least ten genes in a second sample from the subject at a second period of time; c) comparing the gene expression level in step (a) to the amount detected in step (b), wherein the acute rejection is not progressing if the gene expression level of at least five genes selected from the group consisting of CFLAR, DUSP1, IFNGR1, ITGAX, MAPK9, NAMPT, NKTR, PSEN1, RNF130, and RYBP changes over time. In some embodiments, the gene expression level of the at least five genes changes over time to become statistically similar to a gene expression profile indicative of an acute rejection response. In some embodiments, the gene expression level of the at least five genes changes over time to become statistically different to a gene expression profile indicative of an absence of an acute rejection response. Serial samples can be procured and measured by the methods described herein to monitor the progression of an acute rejection response. For example, a sample can be procured and measured at a first period of time, second period of time, third period of time, fourth period of time, etc. as necessary to monitor the progression of an acute rejection in a subject of interest. It is contemplated that the serial samples can be compared to each other in any combination without limitation. The samples can be collected at any moment or time or any time during the course of treatment. For example, a sample can be collected at a first period of time before initiation of treatment for acute rejection response and at a second moment (or third moment or fourth moment, etc.) in time after initiation of an acute rejection response to monitor for any improvement in the acute rejection response upon treatment.

In some aspects, provided herein is a method for identifying a subject who has received a solid organ allograft in need of treatment of an acute rejection response, wherein the method comprises: a) detecting a gene expression level for at least ten genes in a sample from the subject, wherein the at least ten genes comprise CFLAR, DUSP1, IFNGR1, ITGAX, MAPK9, NAMPT, NKTR, PSEN1, RNF130, and RYBP; b) comparing the gene expression level to a reference expression level of the at least ten genes; and c) determining whether the subject has an acute rejection response based upon a statistical difference or a statistical similarity between the gene expression level and the reference expression level of at least five genes selected from the group consisting of CFLAR, DUSP1, IFNGR1, ITGAX, MAPK9, NAMPT, NKTR, PSEN1, RNF130, and RYBP, thereby identifying the subject in need of treatment of an acute rejection response. A subject identified in need of treatment for an acute rejection response may then seek the proper course of treatment described herein or known in the art. For example, also provided herein are methods of treating an acute rejection response in a subject who has received a solid organ allograft, wherein the method comprises: a) detecting a gene expression level of at least ten genes in a sample from the subject, wherein the at least ten genes comprise CFLAR, DUSP1, IFNGR1, ITGAX, MAPK9, NAMPT, NKTR, PSEN1, RNF130, and RYBP; b) comparing the gene expression level to a reference expression level of the at least ten genes; c) determining the subject has an acute rejection response based upon a statistical difference or a statistical similarity between the gene expression level and the reference expression level of at least five genes selected from the group consisting of CFLAR, DUSP1, IFNGR1, ITGAX, MAPK9, NAMPT, NKTR, PSEN1, RNF130, and RYBP; and d) administering a therapeutically effective amount of one or more of a therapeutic agent to treat the acute rejection response. In some embodiments herein, a method for identifying a subject who has received a solid organ allograft in need of treatment of an acute rejection response comprises an additional step of procuring a sample from the subject who has received a solid organ allograft. In some embodiments herein, a method of treating an acute rejection response in a subject who has received a solid organ allograft comprises an additional step of procuring a sample from the subject who has received a solid organ allograft.

In some aspects, provided herein is a method for analysis of gene expression data obtained from a subject who has received a solid organ allograft for determination of an acute rejection response, the method comprising: a) detecting the expression level for at least ten genes in a sample from the subject, wherein the at least ten genes comprise CFLAR, DUSP1, IFNGR1, ITGAX, MAPK9, NAMPT, NKTR, PSEN1, RNF130, and RYBP, thereby obtaining gene expression data from the subject; b) comparing the gene expression data to a gene expression profile prepared by any method described herein; and c) determining a statistical difference or a statistical similarity between the gene expression data and the gene expression profile of at least five genes selected from the group consisting of CFLAR, DUSP1, IFNGR1, ITGAX, MAPK9, NAMPT, NKTR, PSEN1, RNF130, and RYBP. Also provided herein are methods of comparing gene expression data from a subject who has received a solid organ allograft to a gene expression profile, the method comprising: a) detecting the expression level for at least ten genes, wherein the at least ten genes comprise CFLAR, DUSP1, IFNGR1, ITGAX, MAPK9, NAMPT, NKTR, PSEN1, RNF130, and RYBP in a sample from the subject, thereby obtaining gene expression data from the subject; c) comparing the gene expression data to a gene expression profile prepared by any method described herein; and d) determining a statistical difference or a statistical similarity between the gene expression data and the gene expression profile of at least five genes selected from the group consisting of CFLAR, DUSP1, IFNGR1, ITGAX, MAPK9, NAMPT, NKTR, PSEN1, RNF130, and RYBP. In some embodiments herein, a method for analysis of gene expression data obtained from a subject who has received a solid organ allograft for determination of an acute rejection response comprises an additional step of procuring a sample from the subject who has received a solid organ allograft. In some embodiments herein, a method for comparing gene expression data from a subject who has received a solid organ allograft to a gene expression profile comprises an additional step of procuring a sample from the subject who has received a solid organ allograft.

In any of the methods described herein, the gene expression level of at least 5 genes selected from the group consisting of CFLAR, DUSP1, IFNGR1, ITGAX, MAPK9, NAMPT, NKTR, PSEN1, RNF130, and RYBP) can assess (e.g., predict, diagnose, identify, etc.) an acute rejection response in a subject of interest. Any combination of a minimum set of 5 genes selected from the group consisting of CFLAR, DUSP1, IFNGR1, ITGAX, MAPK9, NAMPT, NKTR, PSEN1, RNF130, and RYBP) can assessed such as, for example, DUSP1, MAPK9, NKTR, NAMPT, and PSEN1; or DUSP1, IFNGR1, MAPK9, NAMPT, and RYBP; or ITGAX, MAPK9, NAMPT, NKTR, PSEN1, etc. as if each and every combination were explicitly written herein. In some embodiments herein, 5 genes selected from the group are assessed in a detecting step described herein. In some embodiments, at least 5, 6, 7, 8, or 9 but no more 10 genes is assessed in a detecting step described herein. In some embodiments, at least 5, 6, 7, 8, 9, 10 or up to 32,000 probes or any equivalent number thereof that can detect any combination of genes in a mammalian genome including at least 5 genes selected from the group consisting of CFLAR, DUSP1, IFNGR1, ITGAX, MAPK9, NAMPT, NKTR, PSEN1, RNF130, and RYBP is assessed in a detecting step described herein.

In some embodiments, the invention provides methods for detection and/or quantitation of circulating nucleic acids or cell-free DNA or cell-free RNA for the diagnosis, prognosis, detection, detection of transplant injury and/or treatment of a transplant status or outcome.

In some embodiments, the circulating nucleic acids or cell-free DNA or cell-free RNA originates from a solid organ allograft from the donor present in the recipient biological fluid as described herein (e.g., blood, urine, or tissue lavage). In some aspects the total circulating nucleic acids or cell-free DNA or cell-free RNA originating from a solid organ allograft from the donor is quantitated. Without being bound by any theory, it is believed that the presence of solid organ allograft cell-free DNA or RNA in biological fluid is indicative of an injury or level of injury to the solid organ allograft and the cell-free DNA or RNA originates from dieing donor organ allograft cells (e.g., apoptotic or necrotic cells). In some aspects, the levels or quantitation of cell-free DNA or cell-free RNA is indicative of the injury status of a solid organ allograft.

In some embodiments, the circulating nucleic acids or cell-free DNA or cell-free RNA originates from recipient blood cells. In some aspects, the circulating nucleic acids or cell-free DNA or cell-free RNA originates from an early blood stem cell (e.g., a hematopoeitic stem cell or hemangioblast), a myeloid progenitor or lymphoid progenitor. In some aspects, the circulating nucleic acids or cell-free DNA or cell-free RNA originates from blood cells comprising mast cells, myeloblasts, basophils, neutrophils, eosinophils, monocytes, macrophages, large granular lymphocytes (e.g., natural killer cells), T lymphocytes, B lymphocytes, or plasma cells. In some aspects, the circulating nucleic acids or cell-free DNA or cell-free RNA originating from the recipient blood cells described herein is quantitated for the expression of at least about 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more genes described herein. In some aspects, the circulating nucleic acids or cell-free DNA or cell-free RNA originating from the recipient blood cells described herein is quantitated for the expression of at least 10 genes selected from the group consisting of CFLAR, DUSP1, IFNGR1, ITGAX, MAPK9, NAMPT, NKTR, PSEN1, RNF130, and RYBP. In some aspects, the gene expression level of at least 5 genes selected from the group consisting of CFLAR, DUSP1, IFNGR1, ITGAX, MAPK9, NAMPT, NKTR, PSEN1, RNF130, and RYBP.

In some embodiments, a genetic fingerprint is generated for the donor organ. This approach allows for a reliable identification of sequences arising solely from the organ transplantation that can be made in a manner that is independent of the genders of donor and recipient.

In some embodiments, both the donor and recipient will be genotyped prior to transplantation. Examples of methods that can be used to genotype the transplant donor and the transplant recipient include, but are not limited to, whole genome sequencing, exome sequencing, or polymorphisms arrays (e.g., SNP arrays). In this way, a set of relevant and distinguishable markers between the two sources is established. In some aspects, the set of markers comprises a set of polymorphic markers. Polymorphic markers include single nucleotide polymorphisms (SNP's), restriction fragment length polymorphisms (RFLP's), short tandem repeats (STRs), variable number of tandem repeats (VNTR's), hypervariable regions, minisatellites, dinucleotide repeats, trinucleotide repeats, tetranucleotide repeats, simple sequence repeats, and insertion elements such as Alu. In some aspects, the set of markers comprises SNPs.

In some embodiments, following transplantation, biological fluids or sample sources described herein can be drawn from the patient and analyzed for specific identifying markers. In some aspects, detection, genotyping, identification and/or quantitation of the donor-specific markers (e.g. polymorphic markers such as SNPs) can be performed using digital PCR, real-time PCR, chips (e.g., SNP chips), high-throughput shotgun sequencing of circulating nucleic acids (e.g. cell-free DNA), as well as other methods known in the art including the methods described herein. The proportion of donor nucleic acids can be monitored over time and an increase in this proportion can be used to determine transplant status or outcome. In some aspects, the proportion, concentration, or percentage of donor cell-free DNA is indicative of a stable or healthy donor organ transplant. In some aspects, the proportion, concentration, or percentage of donor cell-free DNA is indicative of an allograft rejection (e.g., acute AR or chronic AR) or cytomegalovirus (CMV) infection. In some aspects, the proportion, concentration, or percentage of donor cell-free DNA is indicative of general chronic donor organ injury. In some aspects, the proportion, concentration, or percentage of donor cell-free DNA is indicative of an acute allograft rejection. In some aspects, the proportion, concentration, or percentage of donor cell-free DNA is indicative of an acute allograft rejection. In some aspects, the proportion, concentration, or percentage of donor cell-free DNA is indicative of cytomegalovirus (CMV) infection.

In another embodiment, the method to assess the allograft or organ transplant status of an individual (e.g., a human) comprises determining the copy number of Chromosome 1, Chromosome 2, Chromosome 3, Chromosome 4, Chromosome 5, Chromosome 6, Chromosome 7, Chromosome 8, Chromosome 9, Chromosome 10, Chromosome 11, Chromosome 12, Chromosome 13, Chromosome 14, Chromosome 15, Chromosome 16, Chromosome 17, Chromosome 18, Chromosome 19, Chromosome 20, Chromosome 21, Chromosome 22, Chromosome X, and/or Chromosome Y in a urine sample, and comparing the copy number of the chromosome to either a standard copy number of that chromosome in a biological fluid sample from a normal population or to an otherwise predetermined standard level or threshold value, wherein a change in the copy number is indicative of an altered allograft or organ transplant status. If the copy number of the chromosome is determined to be higher than the standard copy number or threshold value, it is indicative of compromised allograft or organ transplant status and acute allograft rejection. If the copy number of the chromosome is determined to be equal or lower than the standard copy number or threshold value, it is indicative of no acute allograft rejection

In another embodiment, the method to assess the allograft or organ transplant status of an individual comprises determining the copy number of any sex chromosome in a biological fluid sample, and comparing the copy number of the chromosome to either a standard copy number of that chromosome in a biological fluid sample from a normal population or to an otherwise pre-determined standard level, wherein a change in the copy number is indicative of an altered allograft or organ transplant status.

In one embodiment, digital PCR can be used to determine the copy number of any chromosome, or the copy number of any autosomal chromosome, or the copy number of any sex chromosome. More specifically digital PCR can be used to determine the copy number of Chromosome 1, Chromosome 2, Chromosome 3, Chromosome 4, Chromosome 5, Chromosome 6, Chromosome 7, Chromosome 8, Chromosome 9, Chromosome 10, Chromosome 11, Chromosome 12, Chromosome 13, Chromosome 14, Chromosome 15, Chromosome 16, Chromosome 17, Chromosome 18, Chromosome 19, Chromosome 20, Chromosome 21, and/or Chromosome 22. Similarly digital PCR can be used to determine the copy number of Chromosome Y or Chromosome X.

In one embodiment, digital PCR can be used to determine the copy number of Chromosome 1 with suitable primers designed to amplify a portion of the EIF2C1 locus on Chromosome 1. In another embodiment, digital PCR can be used to determine the copy number of Chromosome Y with suitable primers designed to amplify a portion of the DYS 14 locus on Chromosome Y.

In some embodiments, the detection, genotyping, identification and/or quantitation of the donor-specific nucleic acids after transplantation (e.g. polymorphic markers such as SNPs) can be performed by sequencing such as whole genome sequencing, exome sequencing, or next generation sequencing methods known in the art.

In some embodiments, the amount of one or more nucleic acids from the transplant donor in a sample from the transplant recipient is used to determine the transplant status or outcome. Thus, in some embodiments, the methods of the invention further comprise quantitating the one or more nucleic acids from the transplant donor. In some embodiments, the amount of one or more nucleic acids from the donor sample is determined as a percentage of the total of the nucleic acids in the sample. In some embodiments, the amount of one or more nucleic acids from the donor sample is determined as a ratio of the total nucleic acids in the sample. In some embodiments, the amount of one or more nucleic acids from the donor sample is determined as a ratio or percentage compared to one or more reference nucleic acids in the sample. For example, the amount of one or more nucleic acids from the transplant donor can be determined to be about 0.01% to about 10% of the total nucleic acids in the sample. Alternatively, the amount of one or more nucleic acids from the transplant donor can be at a ratio of about 1:100 to about 1:10 compared to the total of the nucleic acids in the sample. Further, the amount of one or more nucleic acids from the transplant donor can be determined to be 10% or at a ratio of 1:10 of a reference or housekeeping gene, such as beta-globin. In some embodiments, the amount of one or more nucleic acids from the transplant donor can be determined as a concentration; for example, the amount of one or more nucleic acids from the donor sample can be determined to be from about 0.1 ng/mL to about 1 ug/mL, including all iterations of nucleic acid concentrations within the specified range.

In some embodiments, the amount of one or more nucleic acids from the transplant donor above a predetermined threshold value is indicative of a transplant status or outcome. For example, the normative values for clinically stable post-transplantation patients with no evidence of graft rejection or other pathologies can be determined. An increase in the amount of one or more nucleic acids from the transplant donor above the normative values for clinically stable post-transplantation patients could indicate a change in transplant status or outcome, such as transplant rejection or transplant injury. On the other hand, an amount of one or more nucleic acids from the transplant donor below or at the normative values for clinically stable post-transplantation patients could indicate graft tolerance or graft survival.

In some aspects, provided herein is a method for aiding in the diagnosis of an acute rejection response, predicting an acute rejection response, predicting the likelihood of an acute rejection response, monitoring the progression of an acute rejection response, or identifying a subject in need of treatment of an acute rejection response in a subject who has received a solid organ allograft, the method comprising: a) detecting the ratio, concentration, or percentage of donor cell nucleic acid from a mixture of nucleic acids freely circulating in a sample source (e.g., cell-free DNA or RNA) as described herein, wherein the amount of one or more nucleic acids from the transplant donor above or below a predetermined threshold value; b) detecting a gene expression level for at least ten genes in a sample from the subject, wherein the at least ten genes comprise CFLAR, DUSP1, IFNGR1, ITGAX, MAPK9, NAMPT, NKTR, PSEN1, RNF130, and RYBP; and c) comparing the gene expression level to a reference expression level of the at least ten genes, wherein a statistical difference or a statistical similarity between the gene expression level and the reference expression level of at least five genes selected from the group consisting of CFLAR, DUSP1, IFNGR1, ITGAX, MAPK9, NAMPT, NKTR, PSEN1, RNF130, and RYBP is indicative of a transplant status or outcome.

In some embodiments, the method for aiding in the diagnosis of an acute rejection response, predicting an acute rejection response, predicting the likelihood of an acute rejection response, monitoring the progression of an acute rejection response, or identifying a subject in need of treatment of an acute rejection response in a subject who has received a solid organ allograft comprises: a) detecting the ratio, concentration, or percentage of donor cell nucleic acid from a mixture of nucleic acids freely circulating in a sample source (e.g., cell-free DNA or RNA) as described herein; b) detecting a gene expression level for at least ten genes in a sample from the subject, wherein the at least ten genes comprise CFLAR, DUSP1, IFNGR1, ITGAX, MAPK9, NAMPT, NKTR, PSEN1, RNF130, and RYBP; and c) comparing the gene expression level to a reference expression level obtained from a control sample, wherein the control sample is: (i) from at least one subject with an acute rejection response to a solid organ allograft, or (ii) from at least one subject without an acute rejection response to a solid organ allograft, wherein a statistical similarity for at least five genes selected from the group consisting of CFLAR, DUSP1, IFNGR1, ITGAX, MAPK9, NAMPT, NKTR, PSEN1, RNF130, and RYBP between the sample and the control sample of (i) is indicative of an acute rejection response in a subject and wherein the level of donor cell-free DNA/or RNA is above a threshold amount further indicates the presence of an acute rejection response in the subject; or wherein detection of a statistical similarity for at least five genes selected from the group consisting of CFLAR, DUSP1, IFNGR1, ITGAX, MAPK9, NAMPT, NKTR, PSEN1, RNF130, and RYBP between the sample and the control sample of (ii) indicates no acute rejection response and wherein the level of donor cell-free DNA/or RNA is below a threshold amount further indicates an absence of an acute rejection response in the subject.

In some embodiments, the method for aiding in the diagnosis of an acute rejection response, predicting an acute rejection response, predicting the likelihood of an acute rejection response, monitoring the progression of an acute rejection response, or identifying a subject in need of treatment of an acute rejection response in a subject who has received a solid organ allograft comprises: a) detecting the ratio, concentration, or percentage of donor cell nucleic acid from a mixture of nucleic acids freely circulating in a sample source (e.g., cell-free DNA or RNA) as described herein; b) detecting a gene expression level for at least ten genes in the sample, wherein the at least ten genes comprise CFLAR, DUSP1, IFNGR1, ITGAX, MAPK9, NAMPT, NKTR, PSEN1, RNF130, and RYBP; and c) comparing the gene expression level to a reference expression level obtained from a control sample, wherein the control sample is: (i) from at least one subject with an acute rejection response to a solid organ allograft, or (ii) from at least one subject without an acute rejection response to a solid organ allograft, wherein a statistical difference for at least five genes selected from the group consisting of CFLAR, DUSP1, IFNGR1, ITGAX, MAPK9, NAMPT, NKTR, PSEN1, RNF130, and RYBP between the sample and the control sample of (i) is indicative of an absence of an acute rejection response in a subject and wherein the level of donor cell-free DNA/or RNA is below a threshold amount further indicates an absence of an acute rejection response; or wherein detection of a statistical difference for at least five genes selected from the group consisting of CFLAR, DUSP1, IFNGR1, ITGAX, MAPK9, NAMPT, NKTR, PSEN1, RNF130, and RYBP between the sample and the control sample of (ii) and wherein the level of donor cell-free DNA/or RNA is above a threshold amount indicates an acute rejection response in the subject.

In some aspects, provided herein is a method for aiding in the diagnosis of an acute rejection response, predicting an acute rejection response, predicting the likelihood of an acute rejection response, monitoring the progression of an acute rejection response, or identifying a subject in need of treatment of an acute rejection response in a subject who has received a solid organ allograft, the method comprising: a) detecting a gene expression level for at least ten genes from a mixture of nucleic acids freely circulating in a sample source from the subject (e.g., cell-free DNA or RNA) as described herein, wherein the at least ten genes comprise CFLAR, DUSP1, IFNGR1, ITGAX, MAPK9, NAMPT, NKTR, PSEN1, RNF130, and RYBP; and b) comparing the gene expression level to a reference expression level of the at least ten genes, wherein a statistical difference or a statistical similarity between the gene expression level and the reference expression level of at least five genes selected from the group consisting of CFLAR, DUSP1, IFNGR1, ITGAX, MAPK9, NAMPT, NKTR, PSEN1, RNF130, and RYBP.

In some embodiments, the method for aiding in the diagnosis of an acute rejection response, predicting an acute rejection response, predicting the likelihood of an acute rejection response, monitoring the progression of an acute rejection response, or identifying a subject in need of treatment of an acute rejection response in a subject who has received a solid organ allograft comprises: a) detecting a gene expression level for at least ten genes from a mixture of nucleic acids freely circulating in a sample source from the subject (e.g., cell-free DNA or RNA) as described herein, wherein the at least ten genes comprise CFLAR, DUSP1, IFNGR1, ITGAX, MAPK9, NAMPT, NKTR, PSEN1, RNF130, and RYBP; and b) comparing the gene expression level to a reference expression level obtained from a control sample, wherein the control sample is: (i) from at least one subject with an acute rejection response to a solid organ allograft, or (ii) from at least one subject without an acute rejection response to a solid organ allograft, wherein a statistical similarity for at least five genes selected from the group consisting of CFLAR, DUSP1, IFNGR1, ITGAX, MAPK9, NAMPT, NKTR, PSEN1, RNF130, and RYBP between the sample and the control sample of (i) is indicative of an acute rejection response in a subject or wherein detection of a statistical similarity for at least five genes selected from the group consisting of CFLAR, DUSP1, IFNGR1, ITGAX, MAPK9, NAMPT, NKTR, PSEN1, RNF130, and RYBP between the sample and the control sample of (ii) indicates an absence of an acute rejection response in the subject.

In some embodiments, the method for aiding in the diagnosis of an acute rejection response, predicting an acute rejection response, predicting the likelihood of an acute rejection response, monitoring the progression of an acute rejection response, or identifying a subject in need of treatment of an acute rejection response in a subject who has received a solid organ allograft comprises: a) detecting a gene expression level for at least ten genes from a mixture of nucleic acids freely circulating in a sample source from the subject (e.g., cell-free DNA or RNA) as described herein, wherein the at least ten genes comprise CFLAR, DUSP1, IFNGR1, ITGAX, MAPK9, NAMPT, NKTR, PSEN1, RNF130, and RYBP; and b) comparing the gene expression level to a reference expression level obtained from a control sample, wherein the control sample is: (i) from at least one subject with an acute rejection response to a solid organ allograft, or (ii) from at least one subject without an acute rejection response to a solid organ allograft, wherein a statistical difference for at least five genes selected from the group consisting of CFLAR, DUSP1, IFNGR1, ITGAX, MAPK9, NAMPT, NKTR, PSEN1, RNF130, and RYBP between the sample and the control sample of (i) is indicative of an absence of an acute rejection response in the subject; or wherein detection of a statistical difference for at least five genes selected from the group consisting of CFLAR, DUSP1, IFNGR1, ITGAX, MAPK9, NAMPT, NKTR, PSEN1, RNF130, and RYBP between the sample and the control sample of (ii) indicates an acute rejection response in the subject.

### V. Kits for Assessing an Acute Rejection Response

In some aspects, the invention herein also provides for kits for assessing an acute rejection response in a subject who has received a solid organ allograft. The kit described herein can be useful for carrying out any of the methods described herein. In some embodiments, the kit comprises: a) a gene expression evaluation element for evaluating the level of at least ten genes in a sample from the subject to obtain gene expression data, wherein the at least ten genes comprise CFLAR, DUSP1, IFNGR1, ITGAX, MAPK9, NAMPT, NKTR, PSEN1, RNF130, and RYBP; b) a phenotype determination element, wherein the phenotype determination element is one or more of (i) a gene expression profile indicative of an acute rejection response or (ii) a gene expression profile expression profile indicative of an absence of an acute rejection response; and c) a comparison element for comparing the gene expression data to the gene expression profile of (i) and/or (ii), wherein the comparison element compares the expression of at least five genes selected from the group consisting of CFLAR, DUSP1, IFNGR1, ITGAX, MAPK9, NAMPT, NKTR, PSEN1, RNF130, and RYBP.

The gene expression evaluation described herein can comprise at least one reagent for assaying a sample (e.g., a sample procured from a subject with a solid organ allograft). In some embodiments, the reagent is one or more elected from the group consisting of: a microchip array, an array, a bead, and a nanoparticle. A variety of different array (e.g., microarray) formats or other solid substrates are known in the art. Representative arrays or solid substrates that can be used in the kits described herein include, but are not limited to, those described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; and 5,800,992. An array of probes for an expression product (e.g., a protein) or nucleic acid of the at least 10 genes described herein (e.g., CFLAR, DUSP1, IFNGR1, ITGAX, MAPK9, NAMPT, NKTR, PSEN1, RNF130, and RYBP) is contemplated. In some embodiments, the array comprises probes for at least 5 genes selected from the group consisting of CFLAR, DUSP1, IFNGR1, ITGAX, MAPK9, NAMPT, NKTR, PSEN1, RNF130, and RYBP. For example, probes for the at least 5 genes include probes that detect an expression product or nucleic acids for DUSP1, MAPK9, NKTR, NAMPT, and PSEN1; or DUSP1, IFNGR1, MAPK9, NAMPT, and RYBP; or ITGAX, MAPK9, NAMPT, NKTR, PSEN1, etc. Any combination of probes for the at least 5 genes selected from the group consisting of CFLAR, DUSP1, IFNGR1, ITGAX, MAPK9, NAMPT, NKTR, PSEN1, RNF130, and RYBP is contemplated herein as if it were explicitly written. In some embodiments, the array comprises at least 5, 6, 7, 8, or 9, but no more than 10 probes. In some embodiments, the array comprising at least 5, 6, 7, 8, 9, 10 or up to 32,000 probes or any equivalent number thereof that can detect any combination of genes in a mammalian genome including at least 5 genes selected from the group consisting of CFLAR, DUSP1, IFNGR1, ITGAX, MAPK9, NAMPT, NKTR, PSEN1, RNF130, and RYBP. In some embodiments, the mammalian genome is a non-human genome (e.g., a dog genome, a cat genome, a rat genome, a mouse genome, a primate genome, etc.). In some embodiments, the mammalian genome is a human genome.

In some embodiments, the at least one reagent is one or more of an oligonucleotide of predetermined sequence (e.g., a primer) that is specific for RNA encoded by at least 5 genes selected from the group consisting of: CFLAR, DUSP1, IFNGR1, ITGAX, MAPK9, NAMPT, NKTR, PSEN1, RNF130, and RYBP. In some embodiments, the reagent is one or more of an oligonucleotide of predetermined sequence (e.g., a primer) that is specific for DNA complementary to RNA encoded by at least 5 genes selected from the group consisting of: CFLAR, DUSP1, IFNGR1, ITGAX, MAPK9, NAMPT, NKTR, PSEN1, RNF130, and RYBP. In some embodiments, the reagent is one or more of an antibody specific for a gene expression product (e.g., a protein) by at least 5 genes selected from the group consisting of: CFLAR, DUSP1, IFNGR1, ITGAX, MAPK9, NAMPT, NKTR, PSEN1, RNF130, and RYBP. For example, a panel of antibodies can be used to detect the expression of proteins that are encoded by at least 5 genes selected from the group consisting of: CFLAR, DUSP1, IFNGR1, ITGAX, MAPK9, NAMPT, NKTR, PSEN1, RNF130, and RYBP. In some embodiments, the one or more reagent is a primer for generating target nucleic acids, dNTPs and/or rNTPs which may be provide premixed or separately, gold or silver particles with a characteristic scattering spectra, a labeling reagent (e.g., a fluorescent dye, a biotinylation tag, etc.), a buffer (e.g., a hybridization buffer, washing buffer, etc.), a probe purification reagent (e.g., a spin column), a signal generation and detection reagent (e.g., a chemiluminescence substrate), and other reagents known in the art for detection of nucleic acids or expression products of the genes of interest (e.g., at least 5 of CFLAR, DUSP1, IFNGR1, ITGAX, MAPK9, NAMPT, NKTR, PSEN1, RNF130, and RYBP). In some embodiments, a gene expression evaluation element comprises one or more of any combination of reagents described herein. For example, the gene expression evaluation element can comprise any number of combinations of reagents such as an array, a probe, a buffer, and a signal detection agent. In some embodiments, reagents described herein can be used in a kit described herein for nucleic acid amplification techniques well known in the art such as, but not limited to, PCR, Q-PCR, and RT-PCR.

In some embodiments, one of either the gene specific primers or dNTPs, preferably the dNTPs, will be labeled such that the synthesized cDNAs are labeled. By labeled is meant that the entities comprise a member of a signal producing system and are thus detectable, either directly or through combined action with one or more additional members of a signal producing system. Examples of directly detectable labels include isotopic and fluorescent moieties incorporated into, usually covalently bonded to, a nucleotide monomeric unit, e.g. dNTP or monomeric unit of the primer. Isotopic moieties or labels of interest include 32 P, 33 P, 35 S, 125 I, and the like. Fluorescent moieties or labels of interest include coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g. texas red, tetramethylrhodamine, eosins and erythrosins, cyanine dyes, e.g. Cy3 and Cy5, macrocyclic chelates of lanthanide ions, e.g. quantum Dye™, fluorescent energy transfer dyes, such as thiazole orange-ethidium heterodimer, TOTAB, etc. Labels may also be members of a signal producing system that act in concert with one or more additional members of the same system to provide a detectable signal. Illustrative of such labels are members of a specific binding pair, such as ligands, e.g. biotin, fluorescein, digoxigenin, antigen, polyvalent cations, chelator groups and the like, where the members specifically bind to additional members of the signal producing system, where the additional members provide a detectable signal either directly or indirectly, e.g. antibody conjugated to a fluorescent moiety or an enzymatic moiety capable of converting a substrate to a chromogenic product, e.g. alkaline phosphatase conjugate antibody; and the like. Labeled nucleic acid can also be produced by carrying out PCR in the presence of labeled primers. U.S. Pat. No. 5,994,076 is incorporated by reference solely for its teachings of modified primers and dNTPs thereof.

In some embodiments, the kit comprises a phenotype determination element. As used herein the term phenotype determination element includes a gene expression profile that can be used a reference for determination or comparing gene expression data or gene expression levels. The gene expression profile can be any one of those described herein or obtained (e.g., prepared) by a method described herein. In some embodiments, the gene expression profile is obtained from a sample of at least one subject who has received a solid organ allograft and does not have an acute rejection response. In some embodiments, the gene expression profile is obtained from a sample of at least one subject who has received a solid organ allograft and has an acute rejection response. The phenotype determination element can be used for comparison to the gene expression data from a solid organ allograft recipient in order to assess (e.g., predict the likelihood of) an acute rejection response in the subject who has received a solid organ allograft. In some embodiments, the phenotype determination element is computer-generated. In some embodiments, the comparison of the gene expression data to the gene expression profile is performed by a computer. In some embodiments, the comparison of the gene expression data to the gene expression profile is performed by an individual.

In some embodiments, the kit comprises a comparison element for comparing gene expression data to a gene expression profile described herein, can result in the determination of a statistical similarity or statistical difference between the gene expression data and gene expression profile. For example, comparison of gene expression data of the at least ten genes (e.g., CFLAR, DUSP1, IFNGR1, ITGAX, MAPK9, NAMPT, NKTR, PSEN1, RNF130, and RYBP) described herein from a sample of a subject who has received a solid organ allograft and has biopsy-proven acute rejection response will demonstrate a statistical similarity for at least five genes to a gene expression profile that is indicative of an acute rejection response. Conversely, comparison of gene expression data of the at least ten genes (e.g., CFLAR, DUSP1, IFNGR1, ITGAX, MAPK9, NAMPT, NKTR, PSEN1, RNF130, and RYBP) described herein from a sample of a subject who has received a solid organ allograft and has biopsy-proven acute rejection response will demonstrate a statistical difference for at least five genes to a gene expression profile for the at least ten genes that is indicative of an absence of an acute rejection response. In some aspects, a subject does not need to have a biopsy-proven acute rejection response. The kits contemplated herein can be used to assess an acute rejection response in a subject that has not undergone a biopsy for detection of acute rejection of the transplanted organ. A statistical similarity and/or statistical difference can be assessed with one or more of a 70% or greater sensitivity, specificity, positive predictive value (ppv) and negative predictive value (npv), or any other explicit numerical value described herein for these parameters.

As amenable, kit components described herein may be packaged in a manner customary for use by those of skill in the art. For example, the kit components may be provided in solution or as a liquid dispersion or the like. The different reagents included in an inventive kit may be supplied in a solid (e.g., lyophilized) or liquid form. The kits of the present invention may optionally comprise different containers (e.g., vial, ampoule, test tube, flask or bottle) for each individual buffer and/or reagent. Each component will generally be suitable as an aliquot (e.g., a diluted reagent) in its respective container or provided in a concentrated form. Other containers suitable for conducting certain steps of the disclosed methods may also be provided. The individual containers of the kit are preferably maintained in close confinement for commercial sale.

In some embodiments, the kit further comprises a set of instructions for assessing acute rejection response in a subject who has received a solid organ allograft. In certain embodiments, a kit further comprises instructions for using its components for the diagnosis of solid organ status, solid organ transplant status, solid organ disease, solid organ injury, or solid organ graft rejection in a subject according to a method of the invention. Instructions for using the kit according to methods of the invention may comprise instructions for processing the biological sample from a subject of interest (e.g., subject who has received a solid organ allograft) and/or for performing the test, and/or instructions for interpreting the results.

A kit may also contain a notice in the form prescribed by a governmental agency regulating the manufacture, use or sale of pharmaceuticals or biological products.

### VI. Systems for Assessing an Acute Rejection Response

In some aspects, the invention herein also provides for systems for assessing an acute rejection response in a subject who has received a solid organ allograft. The system described herein can be useful for carrying out any of the methods described herein. In some embodiments, the system comprises: a) a gene expression evaluation element for evaluating the level of at least ten genes in a sample from the subject to obtain gene expression data, wherein the at least ten genes comprise CFLAR, DUSP1, IFNGR1, ITGAX, MAPK9, NAMPT, NKTR, PSEN1, RNF130, and RYBP; b) a phenotype determination element, wherein the phenotype determination element is one or more of (i) a gene expression profile indicative of an acute rejection response or (ii) a gene expression profile expression profile indicative of an absence of an acute rejection response; and c) a comparison element for comparing the gene expression data to the gene expression profile of (i) and/or (ii), wherein the comparison element compares the expression of at least five genes selected from the group consisting of CFLAR, DUSP1, IFNGR1, ITGAX, MAPK9, NAMPT, NKTR, PSEN1, RNF130, and RYBP.

The gene expression evaluation described herein can comprise at least one reagent for assaying a sample (e.g., a sample procured from a subject with a solid organ allograft). In some embodiments, the reagent is one or more elected from the group consisting of: a microchip array, an array, a bead, and a nanoparticle. In some embodiments, an array or solid substrate is one described herein. An array of probes for an expression product (e.g., a protein) or nucleic acid of the at least 10 genes described herein (e.g., CFLAR, DUSP1, IFNGR1, ITGAX, MAPK9, NAMPT, NKTR, PSEN1, RNF130, and RYBP) is contemplated. In some embodiments, the array comprises probes for at least 5 genes selected from the group consisting of CFLAR, DUSP1, IFNGR1, ITGAX, MAPK9, NAMPT, NKTR, PSEN1, RNF130, and RYBP. For example, probes for the at least 5 genes include probes that detect an expression product or nucleic acids for DUSP1, MAPK9, NKTR, NAMPT, and PSEN1; or DUSP1, IFNGR1, MAPK9, NAMPT, and RYBP; or ITGAX, MAPK9, NAMPT, NKTR, PSEN1, etc. Any combination of probes for the at least 5 genes selected from the group consisting of CFLAR, DUSP1, IFNGR1, ITGAX, MAPK9, NAMPT, NKTR, PSEN1, RNF130, and RYBP is contemplated herein as if it were explicitly written. In some embodiments, the array comprises at least 5, 6, 7, 8, or 9, but no more than 10 probes. In some embodiments, the array comprising at least 5, 6, 7, 8, 9, 10 or up to 32,000 probes or any equivalent number thereof that can detect any combination of genes in a mammalian genome including at least 5 genes selected from the group consisting of CFLAR, DUSP1, IFNGR1, ITGAX, MAPK9, NAMPT, NKTR, PSEN1, RNF130, and RYBP. In some embodiments, the mammalian genome is a non-human genome (e.g., a dog genome, a cat genome, a rat genome, a mouse genome, a primate genome, etc.). In some embodiments, the mammalian genome is a human genome.

In some embodiments, the at least one reagent is one or more of an oligonucleotide of predetermined sequence (e.g., a primer) that is specific for RNA encoded by at least 5 genes selected from the group consisting of: CFLAR, DUSP1, IFNGR1, ITGAX, MAPK9, NAMPT, NKTR, PSEN1, RNF130, and RYBP. In some embodiments, the reagent is one or more of an oligonucleotide of predetermined sequence (e.g., a primer) that is specific for DNA complementary to RNA encoded by at least 5 genes selected from the group consisting of: CFLAR, DUSP1, IFNGR1, ITGAX, MAPK9, NAMPT, NKTR, PSEN1, RNF130, and RYBP. In some embodiments, the reagent is one or more of an antibody specific for a gene expression product (e.g., a protein) of at least 5 genes selected from the group consisting of: CFLAR, DUSP1, IFNGR1, ITGAX, MAPK9, NAMPT, NKTR, PSEN1, RNF130, and RYBP. For example, a panel of antibodies can be used to detect the expression of proteins that are encoded by at least 5 genes selected from the group consisting of: CFLAR, DUSP1, IFNGR1, ITGAX, MAPK9, NAMPT, NKTR, PSEN1, RNF130, and RYBP. In some embodiments, a gene expression evaluation element comprises one or more of any combination of reagents described above. For example, the gene expression evaluation element can comprise any number of combinations of reagents such as an array, a probe, a buffer, and a signal detection agent. In some embodiments, reagents described herein can be used in a system described herein for nucleic acid amplification techniques well known in the art such as, but not limited to, PCR, Q-PCR, and RT-PCR.

In some embodiments, the system comprises a phenotype determination element. As used herein the term phenotype determination element includes a gene expression profile that can be used a reference for determination or comparing gene expression data or gene expression levels. The gene expression profile can be any one of those described herein or obtained (e.g., prepared) by a method described herein. In some embodiments, the gene expression profile is obtained from a sample of at least one subject who has received a solid organ allograft and does not have an acute rejection response. In some embodiments, the gene expression profile is obtained from a sample of at least one subject who has received a solid organ allograft and has an acute rejection response. The phenotype determination element can be used for comparison to the gene expression data from a solid organ allograft recipient in order to assess (e.g., predict the likelihood of) an acute rejection response in the subject who has received a solid organ allograft. In some embodiments, the phenotype determination element is computer-generated. In some embodiments, the comparison of the gene expression data to the gene expression profile is performed by a computer. In some embodiments, the comparison of the gene expression data to the gene expression profile is performed by an individual.

In some embodiments, the system comprises a comparison element for comparing gene expression data to a gene expression profile described herein, can result in the determination of a statistical similarity or statistical difference between the gene expression data and gene expression profile. For example, comparison of gene expression data of the at least ten genes (e.g., CFLAR, DUSP1, IFNGR1, ITGAX, MAPK9, NAMPT, NKTR, PSEN1, RNF130, and RYBP) described herein from a sample of a subject who has received a solid organ allograft and has biopsy-proven acute rejection response will demonstrate a statistical similarity for at least five genes to a gene expression profile that is indicative of an acute rejection response. Conversely, comparison of gene expression data of the at least ten genes (e.g., CFLAR, DUSP1, IFNGR1, ITGAX, MAPK9, NAMPT, NKTR, PSEN1, RNF130, and RYBP) described herein from a sample of a subject who has received a solid organ allograft and has biopsy-proven acute rejection response will demonstrate a statistical difference for at least five genes to a gene expression profile for the at least ten genes that is indicative of an absence of an acute rejection response. In some aspects, a subject does not need to have a biopsy-proven acute rejection response. The systems contemplated herein can be used to assess an acute rejection response in a subject that has not undergone a biopsy for detection of acute rejection of the transplanted organ. A statistical similarity and/or statistical difference can be assessed with one or more of a 70% or greater sensitivity, specificity, positive predictive value (ppv) and negative predictive value (npv), or any other explicit numerical value described herein for these parameters.

In some embodiments, the system comprises a computing system. In some embodiments, the computing system comprises one or more computer executable logic (e.g., one or more computer program) that is recorded on a computer readable medium. For example, the computing system can execute some or all of the following functions: (i) controlling isolation of nucleic acids from a sample, (ii) pre-amplifying nucleic acids from the sample, (iii) amplifying specific regions in the sample, (iv) identifying and quantifying nucleic acids in the sample, (v) comparing data as detected from the sample with a reference standard (e.g., a gene expression profile), (vi) determining a solid organ status or clinical outcome, (vi) declaring normal (e.g., absence of an acute rejection response) or abnormal solid organ status (e.g., presence of an cut rejection response) or clinical outcome.

The computer executable logic can work in any computer that may be any of a variety of types of general-purpose computers such as a personal computer, network server, workstation, or other computer platform now or later developed. In some embodiments, a computing system is described comprising a computer usable medium having the computer executable logic (computer software program, including program code) stored therein. The computer executable logic can be executed by a processor, causing the processor to perform functions described herein. In other embodiments, some functions are implemented primarily in hardware using, for example, a hardware state machine. Implementation of the hardware state machine so as to perform the functions described herein will be apparent to those skilled in the relevant arts.

The computing system can be configured to perform any one of the methods described herein. For example, the computing system can provide a method of assessing a solid organ status or clinical outcome in an individual at risk for developing, or suffering from solid organ disease, solid organ injury, solid organ graft injury, or solid organ graft rejection (e.g., acute rejection).

### VII. Compositions for Assessing an Acute Rejection Response

In some aspects, the invention herein also provides for compositions comprising one or more solid surfaces for measuring the level of differentially expressed genes associated with acute rejection in a sample from a subject who has received a solid organ allograft. In some embodiments, the solid surfaces provide for the attachment of RNA of the differentially expressed genes. In some embodiments, the solid surfaces provide for the attachment of cDNA of the differentially expressed genes. In other embodiments, the solid surfaces provide for the attachment of primers for amplification of the differentially expressed genes. In some embodiments, the solid surfaces provide for the attachment of protein encoded by the differentially expressed genes. In certain embodiments, the solid surface allows measurement of at least 1, at least 2, at least 3, at least 4, at least 5, at least 6, at least 7, at least 8, at least 9, but no more than 10 differentially expressed genes. In some embodiments, the solid surface allows measurement of at least 1, 2, 3, 4, 5, 6, 7, 8, 9, 10, 15, 20, 25, 30, 35, 40, 45, 50, 55, 60, 65, 75, 80, 85, 90, 95, 100, 105, or 110 differentially expressed genes. In some embodiments, the solid surface allows for measurement of at least 5, 6, 7, 8, 9, 10 or up to 32,000 probes or any equivalent number thereof that can detect any combination of genes in a mammalian genome including at least 5 genes selected from the group consisting of CFLAR, DUSP1, IFNGR1, ITGAX, MAPK9, NAMPT, NKTR, PSEN1, RNF130, and RYBP. In some embodiments, the solid surface allows measurement of a minimum of 5 genes for assessment of an acute rejection response in a subject of interest (e.g., a subject who has received a solid organ allograft). In some embodiments, the solid surface allows measurement of a minimum of 10 genes for assessment of an acute rejection response in a subject of interest (e.g., a subject who has received a solid organ allograft).

In some embodiments, the invention provides a composition which includes one or more solid surfaces for measurement the gene expression level of at least 5 genes selected from the group consisting of CFLAR, DUSP1, IFNGR1, ITGAX, MAPK9, NAMPT, NKTR, PSEN1, RNF130, and RYBP. In some embodiments, the invention provides a composition which includes one or more solid surfaces for measurement the gene expression level of at least 6 genes selected from the group consisting of CFLAR, DUSP1, IFNGR1, ITGAX, MAPK9, NAMPT, NKTR, PSEN1, RNF130, and RYBP. In some embodiments, the invention provides a composition which includes one or more solid surfaces for measurement the gene expression level of at least 7 genes selected from the group consisting of CFLAR, DUSP1, IFNGR1, ITGAX, MAPK9, NAMPT, NKTR, PSEN1, RNF130, and RYBP. In some embodiments, the invention provides a composition which includes one or more solid surfaces for measurement the gene expression level of at least 8 genes selected from the group consisting of CFLAR, DUSP1, IFNGR1, ITGAX, MAPK9, NAMPT, NKTR, PSEN1, RNF130, and RYBP. In some embodiments, the invention provides a composition which includes one or more solid surfaces for measurement the gene expression level of at least 9 genes selected from the group consisting of CFLAR, DUSP1, IFNGR1, ITGAX, MAPK9, NAMPT, NKTR, PSEN1, RNF130, and RYBP. In some embodiments, the invention provides a composition which includes one or more solid surfaces for measurement the gene expression level of at least 10 genes (i.e., all the genes) selected from the group consisting of CFLAR, DUSP1, IFNGR1, ITGAX, MAPK9, NAMPT, NKTR, PSEN1, RNF130, and RYBP. Any combination of the genes selected from the group consisting of CFLAR, DUSP1, IFNGR1, ITGAX, MAPK9, NAMPT, NKTR, PSEN1, RNF130, and RYBP can be used in any of the embodiments described herein. For example, embodiments that contemplate the use of at least 5 genes include one or more solid surfaces that can measure the gene expression level of DUSP1, MAPK9, NKTR, NAMPT, and PSEN1; or DUSP1, IFNGR1, MAPK9, NAMPT, and RYBP; or ITGAX, MAPK9, NAMPT, NKTR, PSEN1, etc. In this exemplary embodiment, any combination of 5 genes selected from the group consisting of CFLAR, DUSP1, IFNGR1, ITGAX, MAPK9, NAMPT, NKTR, PSEN1, RNF130, and RYBP is contemplated herein as if it were explicitly written herein. In some aspects, the invention provides a composition which includes one or more solid surfaces for the measurement of the gene expression level of at least 5 genes comprising DUSP1, IFNGR1, MAPK9, NAMPT, and RYBP.

The following examples are provided for illustrative purposes. These are intended to show certain aspects and embodiments of the present invention but are not intended to limit the invention in any manner.

### EXAMPLES

From the genes listed in Table 1, a subset of 10 genes was identified that can classify patients as AR or no-AR. The genes disclosed in Table 1 can be used for various methods of diagnosing AR in an individual who has received a solid organ allograft, for selecting patients for treatment, as well as for other uses described herein. In some embodiments, at least about 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, or 43 genes from Table 1 are quantitated in the methods described herein for determining whether a subject has an acute allograft rejection.

TABLE 1
43 Genes identified as significantly differentially altered in AR

| Gene Symbol | Ensembl ID | Entrez ID | Definition | TaqMan assay ID |
|---|---|---|---|---|
| RYBP | ENSG00000163602 | 23429 | RING1 and YY1 binding | Hs00171928_m1 |
| | | | protein | |
| RNF130 | ENSG00000113269 | 55819 | Ring finger protein 130 | Hs00218335_m1 |
| PSEN1 | ENSG00000080815 | 5663 | presenilin 1 | Hs00997789_m1 |
| NKTR | ENSG00000114857 | 4820 | natural killer-tumor | Hs00234637_m1 |
| | | | recognition sequence | |
| NAMPT | ENSG00000105835 | 10135 | Nicotinamide | Hs00237184_m1 |
| | | | phosphoribosyltransferase | |
| MAPK9 | ENSG00000050748 | 5601 | mitogen-activated protein | Hs00177102_m1 |
| | | | kinase 9 | |
| ITGAX | ENSG00000140678 | 3687 | integrin, alpha X | Hs00174217_m1 |
| | | | (complement component | |
| | | | 3 receptor 4subunit) | |
| IFNGR1 | ENSG00000027697 \| | 3459 | interferon gamma | Hs00166223_m1 |
| | LRG_66 | | receptor 1 | |
| DUSP1 | ENSG00000120129 | 1843 | dual specificity | Hs00610256_g1 |
| | | | phosphatase 1 | |
| CFLAR | ENSG00000003402 | 8837 | CASH and FADD-like | Hs00236002_m 1 |
| | | | apoptosis regulator | |
| 5LC25A37 | ENSG00000147454 | 51312 | solute carrier family 25, | Hs00249769_m1 |
| | | | member 37 | |
| RXRA | ENSG00000186350 | 6256 | retinoid X receptor, alpha | Hs01067640_m1 |
| RHEB | ENSG00000106615 | 6009 | Ras homolog enriched in | Hs02858186_m1 |
| | | | brain | |
| RARA | ENSG00000131759 | 5914 | retinoic acid receptor, | Hs00940446_m1 |
| | | | alpha | |
| GZMK | ENSG00000113088 | 3003 | granzyme K (granzyme | Hs00157875_m1 |
| | | | 3; tryptase II) | |
| EPOR | ENSG00000187266 | 2057 | erythropoietin receptor | Hs00959427_m1 |
| CEACAM4 | ENSG00000105352 | 1089 | carcinoembryonic | Hs00156509_m1 |
| | | | antigen-related cell | |
| | | | adhesion molecule 4 | |
| NFE2 | ENSG00000123405 | 4778 | nuclear factor (erythroid- | Hs00232351_m1 |
| | | | derived 2), 45 kDa | |
| MPP1 | ENSG00000130830 | 4354 | membrane protein, | Hs00609971_m1 |
| | | | palmitoylated 1, 55 kDa | |
| MAP2K3 | ENSG00000034152 | 5606 | mitogen-activated protein | Hs00177127_m1 |
| | | | kinase kinase 3 | |
| IL2RB | ENSG00000100385 | 3560 | interleukin 2 receptor, | Hs01081697_m1 |
| | | | beta | |
| FOXP3 | ENSG00000049768 \| | 50943 | forkhead box P3 | Hs00203958_m1 |
| | LRG_62 | | | |
| CXCL10 | ENSG00000169245 | 3627 | chemokine (C-X-C | Hs00171042_m1 |
| | | | motif) ligand 10 | |
| C1orf38 | ENSG00000130775 | 9473 | chromosome 1 open | Hs00985482_m1 |
| | | | reading frame 38 | |
| GZMB | ENSG00000100453 | 3002 | Granzyme B | Hs00188051_m1 |
| ABTB1 | ENSG00000114626 | 80325 | ankyrin repeat and BTB | Hs00261395_m1 |
| | | | (P02) domain containing 1 | |
| IL7R | ENSG00000168685 \| | 3575 | interleukin 7 receptor | Hs00233682_m1 |
| | LRG_74 | | | |
| STAT3 | ENS000000168610 | 6774 | signal transducer and | Hs01047580_m1 |
| | | | activator of transcription | |
| | | | 3 (acute-phase response | |
| | | | factor) | |
| YPEL3 | ENSG00000090238 | 83719 | yippee-like 3 | Hs00368883_m1 |
| | | | (Drosophila) | |
| PFN1 | ENSG00000108518 | 5216 | profilin 1 | Hs00748915 s1 |
| IL7 | ENSG00000104432 | 3574 | interleukin 7 | Hs00174202_m1 |
| PCTP | ENSG00000141179 | 58488 | phosphatidylcholine | Hs00221886_m1 |
| | | | transfer protein | |
| GBP2 | ENSG00000162645 | 2634 | guanylate binding protein | Hs00894837_m1 |
| | | | 2, interferon-inducible | |
| GBP1 | ENSG00000117228 | 2633 | guanylate binding protein | Hs00977005_m1 |
| | | | 1, interferon-inducible, | |
| | | | 67 kDa | |
| ANK1 | ENSG00000029534 | 286 | ankyrin 1, erythrocytic | Hs00986657_m1 |
| INPP5D | ENSG00000168918 | 3635 | inositol polyphosphate-5- | Hs00183290_m1 |
| | | | phosphatase, 145 kDa | |
| CHST11 | ENSG00000171310 | 50515 | Carbohydrate | Hs00218229_m1 |
| | | | (chondroitin 4) | |
| | | | sulfotransferase 11 | |
| TNFRSF1A | ENSG00000067182 \| | 7132 | tumor necrosis factor | Hs01042313_m1 |
| | LRG_193 | | receptor superfamily, | |
| | | | member 1A | |
| LYST | ENSG00000143669 | 1130 | lysosomal trafficking | Hs00915897_m1 |
| | | | regulator | |
| ADAMS | ENSG00000151651 | 101 | ADAM metallopeptidase | Hs00923282_g1 |
| | | | domain 8 | |
| RUNX3 | ENSG00000020633 | 864 | runt-related transcription | Hs00231709_m1 |
| | | | factor 3 | |
| PSMB9 | ENSG00000240065 \| | 5698 | proteasome (prosome, | Hs00544762_m1 |
| | ENSG00000239836 \| | | macropain) subunit, beta | |
| | ENSG00000243958 \| | | type, 9 (large multi- | |
| | ENSG00000243594 \| | | functional peptidase 2) | |
| | ENSG00000243067 \| | | | |
| | ENSG00000243067 \| | | | |
| | ENSG00000242711 \| | | | |
| | ENSG00000240508 \| | | | |
| | ENSG00000240118 | | | |
| ISG20 | ENSG00000172183 | 3669 | interferon stimulated | Hs00158122 ml |
| | | | exonuclease gene 20 kDa | |

### Example 1

### Diagnosis and Prediction of Acute Rejection of Heart Transplant

To determine if the same gene panel that was recently discovered as pertinent for diagnosis of renal transplant rejection could also detect and predict transplant rejection across different solid organs, the 10-gene panel was validated by Q-PCR in 141 blood samples from 45 heart transplant recipients with stable graft function (STA, n=41), acute rejection (AR, n=66), cytomegalovirus infection (CMV, n=12) and samples drawn within 6 months of AR (n=23). A QPCR logistic regression model was built on 32 samples and tested for AR prediction in an independent set of 109 samples. Cardiac allograft vasculopathy (CAV) was scored at serial times up to 4 years post-transplant.

Methods

### Study Population

This study utilized a cohort of 45 consecutive patients undergoing first heart transplantation between January 2002 and May 2005. The clinical profile of the 45 study patients is summarized in Table 2. This cohort was assembled prospectively to study the relationship between cytomegalovirus (CMV) infection and the development of cardiac allograft vasculopathy. Age younger than 10 years, renal dysfunction requiring prolonged dialysis, and inability or unwillingness to provide signed informed consent represented exclusion criteria for study enrollment. All patients gave informed consent to the protocol approved by an institutional review board for studies in human subjects.

TABLE 2
Clinical profile of 45 study patients

| Patient Clinical Variables | | |
|---|---|---|
| Age (years, mean ± SD) | 48.2 ± 17.3 | |
| Sex (% male) | 73% | |
| Race/ethnicity, n (%) | | |
| Caucasian | 36 | (80%) |
| Asian | 1 | (2%) |
| Hispanic | 4 | (9%) |
| African-American | 3 | (7%) |
| Other | 1 | (2%) |
| Primary disease, n (%) | | |
| Ischemic CM | 16 | (36%) |
| Dilated CM | 26 | (58%) |
| Other | 3 | (7%) |
| Diabetes, n (%) | 13 | (29%) |
| Hypertension, n (%) | 45 | (100%) |
| History of Smoking, n (%) | 7 | (16%) |

| | |
|---|---|
| Sample time in months post-transplant | 15.0 ± 10.9 |
| (mean ± SD) | |

### Sample Grading and Collection

All study patients were monitored for acute cellular rejection by surveillance endomyocardial biopsy (EMB) performed at scheduled intervals after transplant: weekly during the first month, biweekly until the 3rd month, monthly until the 6th month, and then at months 9 and 12. Biopsies were graded according to the 1990 International Society for Heart and Lung Transplantation (ISHLT) classification system as 0, 1A, 1B, 2, 3A, and 3B (Table 3). See Billingham et al., J. Heart Transplant, 1990, 9(6):587-93.

TABLE 3
1990 ISHLT Standardized Cardiac Biopsy Grading
Scheme for Acute Cellular Rejection and Corresponding
Number of Samples Studied

| Grade | N = 141 | Histological features |
|---|---|---|
| 0 | 75 (40 + | No rejection |
| | 23* + 12**) | |
| 1, mild | 53 | |
| A- Focal | 31 | Focal perivascular and/or interstitial |
| | | infiltrate without myocyte damage |
| B- Diffuse | 22 | Diffuse infiltrate without myocyte damage |
| 2, moderate | 2 | One focus of infiltrate with associated |
| (focal) | | myocyte damage |
| 3, moderate | 11 | |
| A-Focal | 7 | Multifocal infiltrate with myocyte damage |
| B- Diffuse | 4 | Diffuse infiltrate with myocyte damage |

*23 samples drawn within 6 months prior to or after episodes of acute rejection;
**12 samples drawn from patients with CMV infection (>100 copies of CMV DNA amplified from peripheral blood mononuclear cells)

Whole blood samples were collected and stored at the following time-points post-transplant in the 5P01AI050153-02 Program Project Grant (PPG): day 14; months 1, 2, 3, 4, 5, 6, 9, 12, 16, 20, 24, 28, 32, 36, 40, 44, 48, 52, 56, and 60. From this large pool of samples, only those samples were selected that had adequate RNA quantity (>500 mcg total RNA) and quality (RIN>7) and met one of the following clinical phenotypes: (1) acute rejection, CMV− (AR group); (2) no rejection, CMV− (STA group); and (3) no rejection, CMV+ (CMV group). RIN (RNA integrity Number), was determined by the Agilent Bioanalyzer NanoChip (Agilent, Santa Clara, Calif.). All of the AR blood samples were drawn on the day of the biopsy, just prior to the biopsy procedure. Treatment for AR with pulse corticosteroids+/−anti-thymocyte globulin (ATG) was started on the day after the biopsy. All AR blood samples were thus obtained prior to any treatment intensification of AR. For the AR samples, available samples within a 6 month time frame prior to (pre-) and after (post-) the rejection episode were pulled, based on a previous study on kidney transplant rejection that suggested that the rejection gene signature could identify pre-acute rejection samples within a 6 month time-frame prior to AR. See Sarwal et al., Am J Transplant, 2012, 12(10):2719-29; Naesens et al., Am J Transplant, 2012, 12(10):2730-43; and Le et al., Am J Transplant, 2012, 12(10):2710-8. Multiple samples from a single patient were utilized as long as they had a matched biopsy with conclusive phenotypic diagnosis of AR or STA, with the caveat that the STA sample had to be >1 year distant from the AR episode, so that there was no overlap between STA and pre- and post-AR samples which were only collected within the 6 month timeframe of AR.

Stored blood samples were utilized for this study as follows: 40 samples drawn when EMB showed no evidence of cellular rejection (Grade 0), 31 samples drawn when the EMB was classified as Grade 1A, 22 samples drawn when EMB was classified as Grade 1B, 2 samples drawn when EMB was classified as Grade 2, and 11 samples drawn when EMB was classified as Grade ≧3A. In addition, 12 blood samples were drawn during episodes of CMV reactivation (defined as >100 copies of CMV DNA amplified from peripheral blood mononuclear cells), and 23 samples were drawn within 6 months prior to (n=11), or after an episode of cellular rejection (n=12). For the purposes of this study, stable (STA) was defined as EMB showing no evidence of lymphocytic infiltrate (Grade 0), while acute rejection (AR) was defined as EMB showing evidence of mild-severe lymphocytic infiltrate (Grade 1A-3B). A total of 141 blood samples were drawn from 45 heart transplant recipients.

### Immunosuppressive Drug Regimen

Post-transplant immunosuppression consisted of daclizumab (1 mg/kg IV) administered at the time of transplant surgery and on alternate weeks for a total of five doses; cyclosporine (3-5 mg/kg/day); prednisone initiated at 1 mg/kg/day and tapered to <0.1 mg/kg/day by the 6th post-operative month; and either mycophenolate mofetil 1000-3000 mg daily, or Sirolimus 1-4 mg daily. Changes to this standard immunosuppressive regimen were made on an individualized basis. All patients in whom either donor or recipient was CMV antibody positive received standard CMV prophylaxis consisting of 4 weeks of intravenous ganciclovir. Those recipients who were CMV antibody negative and received a heart from a CMV antibody positive donor received an additional 3 month course of CMV hyperimmune serum and up to 80 days of valganciclovir.

### Total RNA Extraction and Quantitative Real-Time PCR

Peripheral blood (2.5 mL) was collected into PAXgene™ Blood RNA tube (PreAnalytiX/Qiagen, Valencia, Calif., USA) containing lysis buffer and RNA stabilizing solution. Total RNA was extracted with the PAXgene™ Blood RNA System (PreAnalytix/Qiagen, Valencia, Calif., USA) following the manufacturer's instructions, yielding a final concentration of 50-300 ng/μl. A total of 500 ng RNA were reverse transcribed in a 20 μl reaction using the RT² First Strand Kit (SAbioscience), followed by quantitative real-time polymerase chain reaction (Q-PCR) in 384-well plates using the Q-PCR Master Mix (RT² SYBR Green/ROX)(SAbioscience). 5 ng cDNA were added to each 10 μl Q-PCR reaction in duplicated wells. 18s ribosomal RNA was selected as a housekeeping gene and Universal RNA (Stratagene) was used as a plate control. The FoxP3 gene, a previously reported AR biomarker, was included in each plate run to serve as a known gene control. Q-PCR reactions were run in the ABI PRISM 7900HT Sequence Detection System. The relative amount of RNA expression was calculated using a comparative C_{T} method.

### Study Design, Conduct and Statistical Analysis

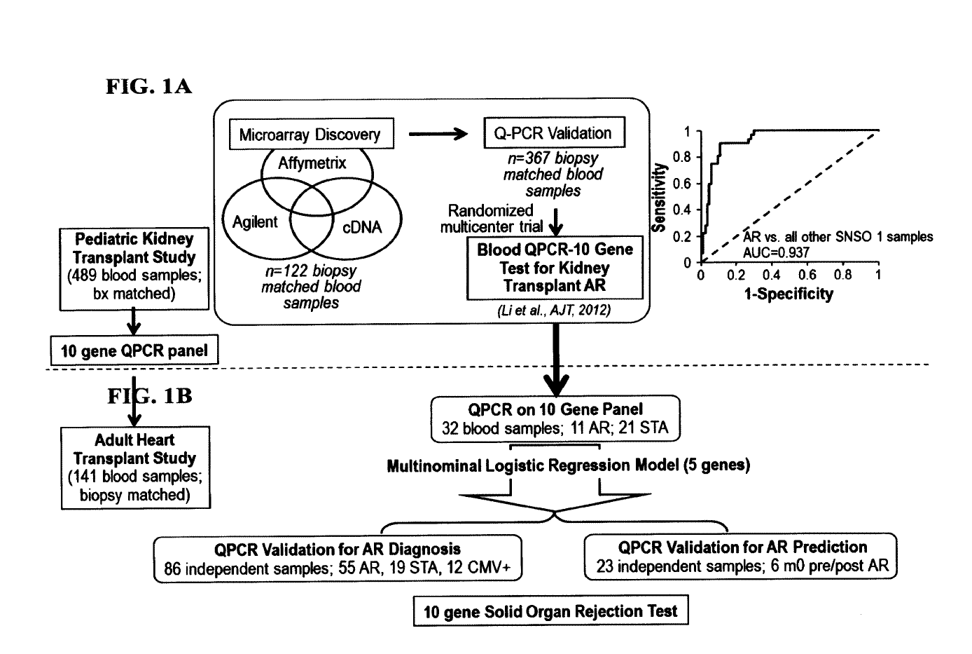

Previous microarray discovery and validation studies were conducted on 489 unique peripheral blood samples from pediatric kidney transplant recipients, with and without biopsy proven acute allograft rejection. See Li et al., Am J Transplant., 2012, 12(10):2710-8. Correlation studies of gene expression profiles in peripheral blood samples of pediatric and young adult renal transplant patients with biopsy-proven acute rejection identified a highly regulated set of 10 genes by microarray analysis (CFLAR, DUSP1, IFNGR1, ITGAX, NAMPT, PSEN1, RNF130, RYBP, MAPK9, and NKTR), and was subsequently validated by Q-PCR (FIG. 1A), which by logistic regression analysis yielded a probability score for acute kidney transplant rejection.

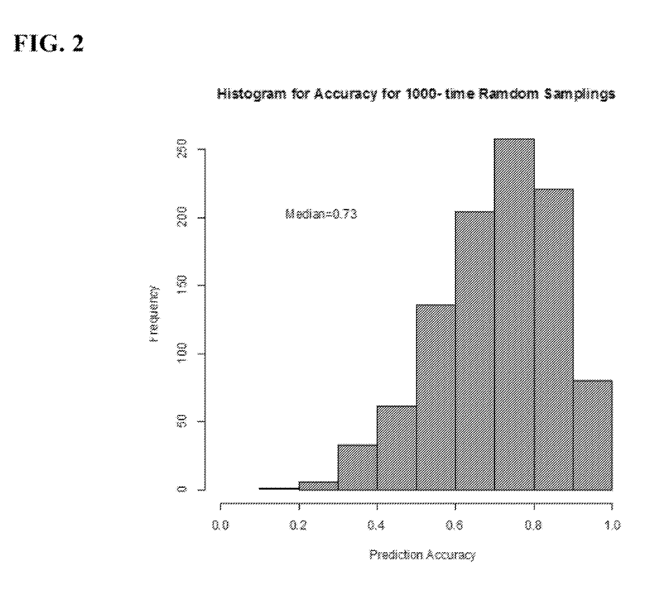

The expression of these 10 genes in peripheral blood samples was assessed to determine if they were also differentially modulated in acute heart transplant rejection. To investigate this, 141 peripheral blood samples were collected from heart transplant recipients at the time of endomyocardial biopsy (FIG. 1B). Histological diagnosis of acute rejection was assessed and graded as previously described. See Billingham et al., J. Heart Transplant, 1990, 9(6):587-93. Given the current clinical practice in most heart transplant centers of only treating Grade 3 AR, only rejection with Grade 3 was included in the discovery set. To confirm the robustness of the signature, the following analytical steps were performed. Firstly, the 32 samples were randomly assigned into training (2/3) and test (1/3) sets for rejection and stable phenotypes; secondly, a logistic regression model was built based on the training set alone; thirdly, the independent test set was classified based on the logistic regression model developed. Using a multinomial logistic regression model, a minimum set of 5 genes was identified that could accurately classify acute rejection blood samples from samples without acute rejection (stable, STA). This procedure was repeated 1000 times and generated a histogram of the accuracy distribution for the test set prediction (FIG. 2). This model was then tested in an independent set of blood samples, again all drawn at the time of endomyocardial biopsy (Q-PCR Prediction for AR Diagnosis; n=86, FIG. 1B), including 55 AR samples (31 Grade 1A, 22 Grade 1B, and 2 Grade 2), 19 samples drawn from patients with no evidence of rejection on biopsy (STA), and 12 blood samples from patients with PCR-confirmed CMV reactivation who had no evidence of cellular rejection (Grade 0). The model was then tested for its ability to segregate samples with acute rejection from those without any evidence of rejection. To evaluate the performance of this model for discriminating acute rejection from CMV infection, (an important cause of graft injury in heart transplant recipients), Q-PCR was performed on the 12 blood samples from patients with documented CMV infection. Finally, serial blood samples were available from 23 patients that were drawn within 6 months prior to or after an episode of biopsy-confirmed acute rejection (Q-PCR Prediction for AR Prediction; n=23; FIG. 1B). The 5-gene model was tested on these samples to ascertain the “rejection score”, to determine whether the gene expression score rose prior to episodes of biopsy-proven acute rejection, and whether the score declined after treatment of the rejection event.

Mean±standard deviations were calculated for patient demographic variables, and mean±standard errors of the means were determined for Q-PCR results. T-tests, chi-square tests, Spearman correlation or Kendall correlation coefficients, and logistic regression models were performed using SAS version 9.2 (SAS institute, Cary, N.C.). The model was built on binary variables of AR or STA based on the fold change of the delta delta Q-PCR CT values which were normalized against 18S and universal RNA. The model was done by SAS 9.2 and reproduced by R 2.15, with likelihood p value of 0.008. All p values were two-sided, and those less than 0.05 were considered significant in all statistical tests. Pearson correlation coefficients were used to evaluate the potential association between continuous variables and gene expression of the 5 genes from Q-PCR and T tests were used to evaluate gene expression levels for the binary variables such as gender and donor age. The hypergeometric test was used to determine whether the proportion of the highly expressed genes in each cell type was statistically significant or not. See Sahai et al. Computers in biology and medicine. 1995, 25(1):35-8. The p-values from hypergeometric test were corrected for multiple hypotheses using Benjamini-Hochberg correction. See Ferreira et al., The International Journal of Biostatistics. 2007; 3(1):Article 11.

Cardiac Allograft Vasculopathy Correlation with AR Prediction

Yearly coronary angiograms were performed with intravascular ultrasound (IVUS), which enables highly accurate measurements of vessel wall thickness, to assess the presence of cardiac allograft vasculopathy (CAV), a common form of chronic rejection after heart transplantation that is characterized by diffuse intimal thickening of the graft coronary arteries. See St Goar et al., Circulation. 1992, 85(3):979-97. Cardiac AR is a known important risk factor for development of CAV. To investigate whether a high peripheral gene-based prediction score of AR would also predict CAV, all study participants were assigned a CAV score from 0-4: 0=no evidence of CAV by angiography or IVUS; 1=coronary artery intimal thickening by IVUS without angiographic disease; 2=coronary artery stenosis<30% by angiography; 3=coronary artery stenosis of 30-70% by angiography; 4=coronary artery stenosis>70% by angiography or placement of an intra-coronary stent. Spearman correlation coefficients were calculated between the gene-based probability scores for AR and subsequent CAV scores to determine whether a high peripheral gene-based prediction score for cardiac AR predicted the subsequent development of CAV.

Results

Development of a 5-Gene Model for Prediction of Acute Cellular Rejection after Heart Transplantation

Selection of the 10 genes for gene expression analysis in this study was done through a multi-platform microarray discovery followed by Q-PCR validation in kidney transplantation (see Li et al., Am J Transplant, 2012, 12(10):2710-8). Among 10,412 common genes probed in all the platforms, 32 genes were selected based on FDR of <5% for differential expression in acute rejection and biological relevance to the immune response; this resulted in a selection of 32 genes (see Li et al., Am J Transplant. 2012, 12(10):2710-8). Validation of an independent set of samples by Q-PCR resulted in the identification of 10 genes that were found to be significantly differentially expressed between rejection and stable graft groups, which were subsequently used for building a classification model by logistic regression. Q-PCR-generated gene expression data for the same set of 10 genes (CFLAR, DUSP1, IFNGR1, ITGAX, NAMPT, PSEN1, RNF130, RYBP, MAPK9, and NKTR), on heart transplant blood samples demonstrated a significant difference between the rejection and non-rejection groups (Table 4). Logistic regression with best subset selection was applied in order to find the minimum number of genes necessary for the proper classification of AR and STA samples. Chi-square score for logistic regression models built using the 10 genes showed that in the dataset used, a model using five genes would have had the same performance as a model using six or more genes (Chi-square of the 5 genes and 10 genes are 9.57 vs. 9.79, respectively). Using only rejection with Grade 3 in the discovery set and by randomly assigning the stable phenotypes, a logistic regression model was built based on the training set alone which was later applied to an independent test set. Using a multinomial logistic regression model, a minimum set of 5 genes of the 10 genes were identified that could accurately classify acute rejection blood samples from samples without acute rejection (stable, STA) with a median accuracy of 0.73 (FIG. 2). The model from the published 5 kidney genes (e.g., DUSP1, MAPK9, NKTR, NAMPT, and PSEN1) did not achieve better performance than one of the best subset of 5 genes selected in the heart dataset (e.g., DUSP1, IFNGR1, MAPK9, NAMPT, and RYBP) which had a chi-square score of 9.57, indicating that different subsets of genes can be chosen from the initial set of 10 genes with equal predictive value for AR.

TABLE 4
Q-PCR-generated gene expression data from heart transplant blood.

| sampleID | ISHLT_ GRADE_VALUE | phenotype | Dataset | CFLAR | DUSP1 | IFNGR1 | ITGAX |
|---|---|---|---|---|---|---|---|
| B1089 | 3A | AR | Training | 7.711152 | 24.70386 | 10.22496 | 116.9991 |
| B1071 | 3A | AR | Training | 11.42395 | 9.805509 | 3.505419 | 31.56389 |
| B1067 | 3A | AR | Training | 20.84984 | 13.47134 | 2.581059 | 92.99754 |
| B1070 | 3B | AR | Training | 3.61112 | 3.648789 | 1.016424 | 8.948424 |
| B1066 | 3A | AR | Training | 1.162941 | 10.26569 | 3.13665 | 4.56094 |
| B1085 | 3A | AR | Training | 18.04705 | 38.57929 | 6.368204 | 92.92482 |
| B1106 | 3B | AR | Training | 4.120875 | 10.16046 | 2.281123 | 22.28621 |
| B1083 | 3A | AR | Training | 11.72964 | 7.24662 | 4.034156 | 60.78818 |
| B1082 | 3A | AR | Training | 4.052988 | 2.368155 | 1.067259 | 11.91928 |
| B1081 | 3B | AR | Training | 13.13089 | 11.93058 | 3.686592 | 50.01764 |
| B1131 | 3B | AR | Training | 34.95491 | 16.95597 | 7.691076 | 176.3681 |
| B1220 | 1B | AR | Test | 3.337111 | 11.06278 | 4.57641 | 84.37681 |
| B1200 | 1B | AR | Test | 5.88386 | 10.22044 | 8.085856 | 91.12854 |
| B1237 | 1B | AR | Test | 5.186804 | 8.926812 | 4.652206 | 49.3076 |
| B1221 | 1B | AR | Test | 4.435522 | 3.334091 | 3.366293 | 31.38964 |
| B1223 | 1B | AR | Test | 8.584576 | 12.90507 | 6.127419 | 79.61683 |
| B1206 | 1B | AR | Test | 2.770918 | 10.52858 | 2.560746 | 51.63528 |
| B1226 | 1B | AR | Test | 13.24892 | 22.99231 | 14.28003 | 59.1821 |
| B1217 | 1B | AR | Test | 5.328016 | 5.164936 | 2.972509 | 32.05989 |
| B1244 | 1B | AR | Test | 6.801862 | 4.756161 | 3.9052 | 36.28177 |
| B1211 | 1B | AR | Test | 4.60678 | 3.226322 | 1.833539 | 22.30024 |
| B1229 | 1B | AR | Test | 11.35244 | 7.452524 | 3.861753 | 50.56433 |
| B1234 | 1B | AR | Test | 7.241413 | 3.874727 | 2.009663 | 16.60642 |
| B1222 | 1B | AR | Test | 16.57769 | 10.93821 | 7.750925 | 82.13767 |
| B1209 | 1B | AR | Test | 31.54585 | 14.2228 | 10.26 | 100.4558 |
| B1134 | 1B | AR | Test | 16.62447 | 17.81617 | 7.322199 | 153.3019 |
| B1105 | 1B | AR | Test | 0.633245 | 3.65855 | 1.235447 | 12.63567 |
| B1224 | 1B | AR | Test | 3.712871 | 10.05851 | 6.27729 | 68.0445 |
| B1240 | 1B | AR | Test | 6.177627 | 7.466606 | 5.694839 | 62.16004 |
| B1233 | 1B | AR | Test | 0.245039 | 2.225863 | 1.166281 | 20.99855 |
| B1219 | 1B | AR | Test | 6.907029 | 30.9417 | 8.336521 | 73.9696 |
| B1202 | 1B | AR | Test | 3.820468 | 9.07593 | 3.394087 | 51.61533 |
| B1194 | 1B | AR | Test | 3.072669 | 3.43038 | 1.173214 | 22.52254 |
| B1155 | 1A | AR | Test | 7.779675 | 4.54562 | 5.068853 | 44.23734 |
| B1095 | 1A | AR | Test | 10.1912 | 0.811043 | 2.556661 | 27.45907 |
| B1114 | 1A | AR | Test | 74.11056 | 58.44576 | 17.05965 | 173.2206 |
| B1133 | 1A | AR | Test | 44.69475 | 30.89833 | 6.652047 | 183.2169 |
| B1116 | 1A | AR | Test | 35.78131 | 32.19526 | 4.020653 | 63.95863 |
| B1135 | 1A | AR | Test | 11.54564 | 9.705924 | 1.400786 | 21.1469 |
| B1147 | 1A | AR | Test | 10.92119 | 10.17443 | 2.635358 | 28.38341 |
| B1110 | 1A | AR | Test | 12.77068 | 18.37126 | 2.748576 | 36.79255 |
| B1126 | 1A | AR | Test | 14.12097 | 12.47887 | 6.395004 | 59.21574 |
| B1120 | 1A | AR | Test | 13.68652 | 20.5327 | 3.757681 | 47.12139 |
| B1197 | 1A | AR | Test | 6.882121 | 9.697999 | 6.697654 | 72.06716 |
| B1203 | 1A | AR | Test | 1.934487 | 3.782179 | 1.683133 | 64.96501 |
| B1242 | 1A | AR | Test | 7.475786 | 4.515006 | 3.689105 | 33.55016 |
| B1239 | 1A | AR | Test | 11.17138 | 4.732797 | 3.991956 | 35.51787 |
| B1245 | 1A | AR | Test | 14.24886 | 20.70765 | 5.07578 | 40.16485 |
| B1232 | 1A | AR | Test | 6.442836 | 4.446694 | 6.466296 | 54.83927 |
| B1235 | 1A | AR | Test | 11.66024 | 7.152108 | 5.106672 | 80.27248 |
| B1195 | 1A | AR | Test | 18.08233 | 13.67336 | 6.099048 | 81.63023 |
| B1241 | 1A | AR | Test | 5.565571 | 5.341549 | 3.864104 | 22.66132 |
| B1201 | 1A | AR | Test | 6.19168 | 9.93457 | 2.651732 | 105.54 |
| B1204 | 1A | AR | Test | 7.390819 | 5.112927 | 3.717346 | 170.155 |
| B1215 | 1A | AR | Test | 7.605912 | 7.837573 | 10.27233 | 41.87237 |
| B1231 | 1A | AR | Test | 13.94697 | 14.27215 | 8.238553 | 170.2605 |
| B1230 | 1A | AR | Test | 33.51527 | 25.27233 | 6.575328 | 502.2144 |
| B1236 | 1A | AR | Test | 13.44536 | 9.415714 | 3.922447 | 160.0577 |
| B1243 | 1A | AR | Test | 15.87759 | 18.43939 | 7.942631 | 395.0459 |
| B1218 | 1A | AR | Test | 14.73143 | 14.64351 | 5.520445 | 272.901 |
| B1213 | 1A | AR | Test | 4.174427 | 3.145508 | 1.80822 | 99.55778 |
| B1207 | 1A | AR | Test | 15.8274 | 15.13008 | 7.737451 | 375.2138 |
| B1199 | 1A | AR | Test | 5.050867 | 2.077487 | 1.549909 | 113.9826 |
| B1210 | 1A | AR | Test | 22.13119 | 21.55806 | 10.41656 | 414.2641 |
| B1122 | 2 | AR | Test | 0.784776 | 2.797989 | 0.681671 | 213.7027 |
| B1127 | 2 | AR | Test | 3.327326 | 7.505811 | 1.412977 | 14.73504 |
| B1118 | non | STA | Training | 8.779946 | 5.618998 | 2.249942 | 27.63918 |
| B1159 | non | STA | Training | 2.62158 | 3.60964 | 1.126775 | 12.45246 |
| B1178 | non | STA | Training | 0.425642 | 4.259667 | 1.13048 | 2.115642 |
| B1164 | non | STA | Training | 0.155839 | 0.082072 | 3.542066 | 8.552627 |
| B1163 | non | STA | Training | 0.317723 | 6.340654 | 0.573934 | 39.26122 |
| B1180 | non | STA | Training | 2.234396 | 6.310838 | 1.255138 | 2.78667 |
| B1145 | non | STA | Training | 15.28875 | 20.29824 | 0.10637 | 44.00985 |
| B1172 | non | STA | Training | 11.98267 | 52.43083 | 15.72334 | 85.73477 |
| B1139 | non | STA | Training | 9.667108 | 14.17632 | 3.677703 | 31.68436 |
| B1142 | non | STA | Training | 21.9079 | 27.79951 | 5.954259 | 50.15595 |
| B1160 | non | STA | Training | 8.274857 | 7.8144 | 8.216873 | 175.6161 |
| B1182 | non | STA | Training | 21.54984 | 29.84839 | 6.408346 | 58.79948 |
| B1161 | non | STA | Training | 3.037298 | 4.896813 | 0.434703 | 9.901112 |
| B1143 | non | STA | Training | 24.69773 | 26.4105 | 3.346916 | 53.21028 |
| B1157 | non | STA | Training | 10.55745 | 10.46182 | 8.070368 | 290.7426 |
| B1186 | non | STA | Training | 30.06354 | 42.25236 | 18.75126 | 125.5779 |
| B1062 | non | STA | Training | 11.1436 | 16.27189 | 7.197909 | 37.06163 |
| B1174 | non | STA | Training | 10.67154 | 39.08776 | 3.336284 | 11.8118 |
| B1179 | non | STA | Training | 4.271833 | 0.168905 | 11.94789 | 2.285093 |
| B1185 | non | STA | Training | 6.332156 | 67.34155 | 10.97247 | 519.5645 |
| B1176 | non | STA | Training | 6.935211 | 10.31786 | 3.490088 | 343.1695 |
| B1115 | non | STA | Test | 1.404346 | 9.293813 | 2.105308 | 11.63071 |
| B1151 | non | STA | Test | 15.43739 | 7.848404 | 4.526447 | 30.71739 |
| B1130 | non | STA | Test | 9.923928 | 19.25282 | 2.438241 | 26.41593 |
| B1140 | non | STA | Test | 6.525045 | 3.82099 | 0.960687 | 16.15546 |
| B1162 | non | STA | Test | 16.16215 | 13.64395 | 13.94932 | 384.3146 |
| B1158 | non | STA | Test | 6.33209 | 8.900347 | 3.861188 | 52.7679 |
| B1165 | non | STA | Test | 0.122285 | 12.59227 | 5.431923 | 0.125025 |
| B1170 | non | STA | Test | 0.840344 | 16.12298 | 1.20499 | 12.30505 |
| B1166 | non | STA | Test | 3.732384 | 9.971459 | 4.001248 | 0.969732 |
| B1169 | non | STA | Test | 3.251895 | 8.552591 | 13.90065 | 115.8845 |
| B1181 | non | STA | Test | 5.250914 | 10.30825 | 3.264001 | 182.7749 |
| B1156 | non | STA | Test | 24.23744 | 36.46069 | 32.80154 | 207.7268 |
| B1077 | non | STA | Test | 33.57873 | 35.74105 | 11.34111 | 120.8858 |
| B1183 | non | STA | Test | 5.069566 | 9.482277 | 2.369708 | 0.69514 |
| B1171 | non | STA | Test | 7.875609 | 5.957243 | 2.96278 | 314.5556 |
| B1173 | non | STA | Test | 6.706174 | 43.68772 | 12.47679 | 89.18778 |
| B1177 | non | STA | Test | 17.02786 | 45.27149 | 18.08943 | 137.6108 |
| B1175 | non | STA | Test | 26.59734 | 30.17961 | 11.87879 | 92.75281 |
| B1144 | non | STA | Test | 27.61815 | 48.02947 | 5.940836 | 40.11563 |
| B1091 | non | CMV | Test | 4.535554 | 3.76773 | 1.10987 | 18.01923 |
| B1119 | non | CMV | Test | 3.294743 | 8.587594 | 1.354155 | 13.34769 |
| B1093 | non | CMV | Test | 5.749424 | 6.849858 | 3.417045 | 35.21701 |
| B1090 | non | CMV | Test | 12.23422 | 14.08388 | 6.941803 | 27.40016 |
| B1086 | non | CMV | Test | 8.879896 | 10.07079 | 6.211017 | 28.7456 |
| B1088 | non | CMV | Test | 6.828065 | 10.98423 | 3.241551 | 17.68619 |
| B1080 | non | CMV | Test | 7.598403 | 13.05664 | 4.732203 | 37.27238 |
| B1069 | non | CMV | Test | 1.464771 | 1.785848 | 0.82992 | 5.918853 |
| B1167 | non | CMV | Test | 9.719102 | 34.9588 | 11.5351 | 111.1138 |
| B1146 | non | CMV | Test | 9.677315 | 12.7265 | 5.151273 | 18.84634 |
| B1148 | non | CMV | Test | 36.10418 | 89.08459 | 27.61556 | 335.7691 |
| B1128 | non | CMV | Test | 9.006395 | 13.94879 | 4.202084 | 27.60979 |
| | non | 3Apost2m | Test | 6.495557 | 4.735271 | 3.463484 | 13.82141 |
| B1098 | non | 3Apost1m | Test | 5.269329 | 4.561621 | 6.064818 | 77.78228 |
| B1097 | non | 3Apost2m | Test | 2.450185 | 2.615375 | 0.91808 | 12.61666 |
| B1099 | non | 1Apost2m | Test | 37.25369 | 32.99281 | 8.119866 | 136.0892 |
| B1150 | non | 3Apost2m | Test | 2.323293 | 4.015981 | 1.062502 | 19.64132 |
| B1113 | non | 2post1m | Test | 14.33143 | 19.35439 | 3.97587 | 64.87308 |
| B1109 | non | 3Apre5m | Test | 37.89388 | 101.46 | 20.07613 | 107.8008 |
| B1096 | non | 1Apost1m | Test | 10.8244 | 5.440954 | 1.993404 | 28.53948 |
| B1123 | non | 1Apost6m | Test | 7.703055 | 1.30126 | 0.065519 | 0.348803 |
| B1104 | non | 3Apre4m | Test | 109.7267 | 65.95292 | 20.61507 | 260.7134 |
| B1184 | non | 1Bpost3m | Test | 7.162972 | 8.000133 | 5.429576 | 50.48208 |
| B1205 | non | 1Bpost1m | Test | 6.330102 | 4.959804 | 3.166318 | 66.87319 |
| B1196 | non | 1Bpost1m | Test | 5.688352 | 3.860921 | 2.910451 | 27.13006 |
| B1225 | non | 1Bpost4m | Test | 7.395552 | 6.708234 | 4.044025 | 48.49051 |
| B1212 | non | 1Apre1m | Test | 1.032623 | 10.05205 | 6.461437 | 83.04633 |
| B1214 | non | 1Bpre2m | Test | 7.8796 | 13.19149 | 6.36475 | 76.01916 |
| B1198 | non | 1Bpre6m | Test | 12.10062 | 5.516329 | 4.825472 | 51.45915 |
| B1238 | non | 1Bpre3m | Test | 18.49784 | 9.348754 | 8.34005 | 77.84714 |
| B1208 | non | 1Apost3m | Test | 37.75445 | 24.05617 | 17.48718 | 358.4977 |
| B1141 | non | 1Apost3m | Test | 3.609079 | 4.861566 | 3.133145 | 66.63832 |
| B1227 | non | 1Apost5m | Test | 5.117155 | 4.065135 | 3.05792 | 79.2832 |
| B1228 | non | 1Apost6m | Test | 1.878948 | 1.334083 | 1.692222 | 82.89953 |
| B1216 | non | 1Apost3m | Test | 5.457277 | 4.21851 | 3.151294 | 121.2605 |
| | | mean STA | | 11.45756 | 19.74302 | 8.07921 | 96.71535 |
| | | mean AR | | 11.84784 | 12.01163 | 4.906848 | 93.05438 |
| | | mean CMV | | 9.591005 | 18.32544 | 6.361798 | 56.41218 |
| | | mean nonAR | | 10.45581 | 19.05426 | 6.679994 | 85.5129 |
| | | (cmv + sta) | | | | | |
| | | ttest | | 0.471776 | 0.014707 | 0.085174 | 0.711451 |
| | | | | CFLAR | DUSP1 | IFNGR1 | ITGAX |
| | | difference | | 0.390284 | −7.7314 | −3.17236 | −3.66097 |
| | | AR-STA | | | | | |

| sampleID | MAPK9 | NKTR | NAMPT | PSEN1 | RNF130 | RYBP | AR_probe |
|---|---|---|---|---|---|---|---|
| B1089 | 1.674933 | 2.801741 | 3.102845 | 10.80642 | 12.33167 | 1.465284 | 89% |
| B1071 | 0.193249 | 0.311341 | 1.802807 | 2.09308 | 2.714305 | 0.427623 | 69% |
| B1067 | 0.187717 | 1.022812 | 3.441238 | 4.871492 | 5.959209 | 0.94576 | 89% |
| B1070 | 0.056196 | 0.173694 | 1.169138 | 1.666177 | 3.97221 | 0.274871 | 73% |
| B1066 | 0.029604 | 0.102522 | 0.117336 | 0.442164 | 1.632985 | 1.255556 | 3% |
| B1085 | 0.064669 | 0.023349 | 5.190473 | 5.735718 | 7.972854 | 0.721641 | 63% |
| B1106 | 0.087121 | 0.171503 | 0.519171 | 0.883 | 0.603275 | 0.227636 | 29% |
| B1083 | 0.185573 | 0.801212 | 3.531784 | 3.409291 | 5.524546 | 0.447922 | 97% |
| B1082 | 0.096893 | 0.27995 | 0.22072 | 0.807174 | 2.113674 | 0.189948 | 51% |
| B1081 | 0.117203 | 0.35048 | 1.105446 | 2.397201 | 3.980212 | 0.262945 | 38% |
| B1131 | 0.62788 | 3.594386 | 7.039901 | 13.21925 | 16.74023 | 1.253352 | 100% |
| B1220 | 0.065339 | 0.3831 | 2.896725 | 4.828726 | 2.298822 | 0.580885 | 80% |
| B1200 | 0.295495 | 1.059941 | 13.71986 | 5.379414 | 6.899014 | 1.424889 | 100% |
| B1237 | 0.183377 | 0.804959 | 6.931884 | 3.689638 | 3.646079 | 0.437771 | 100% |
| B1221 | 0.2166 | 1.416668 | 2.215015 | 3.953927 | 3.766223 | 0.561163 | 89% |
| B1223 | 0.165469 | 2.011847 | 9.302794 | 5.21686 | 6.380408 | 0.847735 | 100% |
| B1206 | 0.21061 | 1.387356 | 10.57022 | 4.941249 | 7.580496 | 0.853772 | 100% |
| B1226 | 0.13936 | 1.116707 | 30.24007 | 4.52065 | 7.211944 | 1.163931 | 100% |
| B1217 | 0.119423 | 0.477548 | 4.45498 | 1.900324 | 2.477495 | 0.443315 | 99% |
| B1244 | 0.261319 | 1.379051 | 8.053492 | 2.358955 | 3.917345 | 0.629449 | 100% |
| B1211 | 0.103964 | 0.715572 | 2.509284 | 1.621571 | 2.85924 | 0.402797 | 94% |
| B1229 | 0.182201 | 1.54986 | 4.877818 | 3.486457 | 3.99708 | 0.636361 | 99% |
| B1234 | 0.093899 | 0.451868 | 1.236953 | 2.165573 | 2.456455 | 0.220184 | 75% |
| B1222 | 0.604752 | 2.38648 | 6.690915 | 4.405292 | 9.3181 | 0.95576 | 100% |
| B1209 | 0.609233 | 4.054422 | 12.31935 | 7.768627 | 8.779369 | 1.538447 | 100% |
| B1134 | 0.718396 | 1.189255 | 7.422185 | 5.291569 | 6.380501 | 0.516357 | 100% |
| B1105 | 0.032149 | 0.169637 | 1.555372 | 0.86863 | 2.600172 | 0.188698 | 83% |
| B1224 | 0.102305 | 0.907375 | 4.887554 | 5.09327 | 3.066878 | 0.902708 | 98% |
| B1240 | 0.229469 | 1.463394 | 8.06585 | 5.307777 | 5.718072 | 0.626419 | 100% |
| B1233 | 0.040129 | 0.143358 | 2.219198 | 0.422343 | 0.417062 | 0.131119 | 95% |
| B1219 | 0.13579 | 0.948996 | 9.17464 | 6.254138 | 6.795067 | 0.784857 | 100% |
| B1202 | 0.113117 | 0.650353 | 8.725798 | 2.62008 | 5.434404 | 0.557015 | 100% |
| B1194 | 0.057946 | 0.306943 | 0.697936 | 1.734175 | 1.390005 | 0.42308 | 52% |
| B1155 | 0.266679 | 1.99169 | 7.114526 | 3.633415 | 6.542948 | 3.457728 | 99% |
| B1095 | 0.091937 | 0.576727 | 4.061557 | 3.45835 | 3.576206 | 0.39988 | 99% |
| B1114 | 0.350025 | 1.619936 | 2.640083 | 12.38961 | 0.598067 | 2.838161 | 0% |
| B1133 | 0.282317 | 1.088561 | 7.109689 | 6.266792 | 6.180516 | 0.651574 | 99% |
| B1116 | 0.312836 | 0.435426 | 1.291749 | 6.774339 | 4.351762 | 0.785233 | 4% |
| B1135 | 0.087794 | 0.804519 | 0.559041 | 2.0421 | 4.072297 | 0.34713 | 32% |
| B1147 | 0.439447 | 1.176272 | 1.366379 | 3.170187 | 3.85941 | 0.540329 | 70% |
| B1110 | 0.090154 | 0.299248 | 0.526747 | 1.87669 | 2.763173 | 0.290647 | 11% |
| B1126 | 0.194205 | 0.76816 | 1.813038 | 3.06684 | 5.392172 | 0.283654 | 52% |
| B1120 | 0.082379 | 0.469954 | 3.225065 | 2.798383 | 5.59856 | 0.632928 | 68% |
| B1197 | 0.213682 | 1.26693 | 9.611213 | 4.636799 | 5.035586 | 1.228775 | 100% |
| B1203 | 0.056729 | 0.257763 | 11.26579 | 1.863178 | 3.729574 | 0.199616 | 100% |
| B1242 | 0.33463 | 1.360524 | 5.726105 | 2.725303 | 2.804933 | 0.549919 | 100% |
| B1239 | 0.272001 | 1.38267 | 4.408472 | 1.968016 | 3.413822 | 0.45998 | 100% |
| B1245 | 0.160828 | 0.55877 | 4.181216 | 3.528767 | 3.757407 | 0.593078 | 90% |
| B1232 | 0.386602 | 2.165579 | 7.937268 | 5.50935 | 6.591214 | 0.941226 | 100% |
| B1235 | 0.246318 | 1.491911 | 10.99251 | 3.865124 | 4.845716 | 0.779828 | 100% |
| B1195 | 0.454413 | 0.982837 | 4.691867 | 5.431336 | 6.572762 | 1.241292 | 97% |
| B1241 | 0.076785 | 0.504676 | 3.20627 | 2.107103 | 3.704716 | 0.292445 | 96% |
| B1201 | 0.122763 | 0.311888 | 3.692038 | 2.305488 | 4.519657 | 0.603352 | 96% |
| B1204 | 0.131953 | 0.622812 | 2.606338 | 2.037879 | 4.758998 | 0.255518 | 93% |
| B1215 | 0.094256 | 0.707699 | 7.553933 | 1.855572 | 4.39615 | 0.705368 | 100% |
| B1231 | 0.145421 | 0.923382 | 2.082673 | 4.776579 | 7.980089 | 0.620764 | 31% |
| B1230 | 0.191789 | 0.901034 | 4.327394 | 10.313 | 10.44203 | 0.776194 | 78% |
| B1236 | 0.060997 | 0.310513 | 1.846733 | 2.902449 | 2.41469 | 0.362371 | 63% |
| B1243 | 0.119948 | 0.448339 | 6.583241 | 3.31519 | 4.44368 | 0.212017 | 100% |
| B1218 | 0.176993 | 0.709509 | 8.301143 | 4.893001 | 7.04047 | 0.78362 | 100% |
| B1213 | 0.053424 | 0.388625 | 4.295702 | 1.231234 | 1.877519 | 0.232473 | 100% |
| B1207 | 0.196815 | 1.459636 | 1.524464 | 4.263445 | 4.105232 | 1.005568 | 11% |
| B1199 | 0.083422 | 0.476263 | 3.603988 | 1.799176 | 1.236151 | 0.23052 | 99% |
| B1210 | 0.254883 | 0.155408 | 7.097006 | 4.364342 | 6.502833 | 0.439009 | 100% |
| B1122 | 0.050992 | 0.138074 | 0.919313 | 6.594689 | 12.24026 | 4.501962 | 0% |
| B1127 | 0.02052 | 0.106878 | 0.965577 | 0.712192 | 0.588974 | 0.108207 | 57% |
| B1118 | 0.160556 | 0.806407 | 1.131171 | 1.632763 | 3.022538 | 0.360029 | 65% |
| B1159 | 0.084 | 0.122634 | 0.127249 | 2.065511 | 3.142104 | 0.465611 | 32% |
| B1178 | 0.021765 | 0.024619 | 0.000206 | 1.591036 | 0.95686 | 0.241694 | 30% |
| B1164 | 0.005407 | 0.003669 | 0.021865 | 4.920702 | 3.604779 | 0.429215 | 25% |
| B1163 | 0.044549 | 0.059261 | 0.105614 | 0.791999 | 2.177583 | 0.235183 | 32% |
| B1180 | 0.020015 | 0.011538 | 0.075668 | 0.800815 | 0.746158 | 0.153863 | 29% |
| B1145 | 0.245783 | 0.639387 | 1.560376 | 4.062387 | 6.565499 | 0.602846 | 41% |
| B1172 | 0.162751 | 0.47059 | 8.064967 | 9.299493 | 15.62499 | 2.198017 | 26% |
| B1139 | 0.103818 | 0.575037 | 0.99134 | 1.978451 | 3.669211 | 0.179232 | 29% |
| B1142 | 0.145809 | 0.42915 | 2.011309 | 5.5933 | 1.755129 | 0.294175 | 15% |
| B1160 | 0.503628 | 2.881909 | 2.82356 | 5.863384 | 11.15493 | 6.08565 | 0% |
| B1182 | 0.154347 | 0.032242 | 2.883126 | 9.675499 | 13.78015 | 1.360512 | 8% |
| B1161 | 0.164245 | 0.139738 | 0.098116 | 1.463826 | 4.259019 | 0.504756 | 35% |
| B1143 | 0.135212 | 0.324109 | 0.372247 | 5.233656 | 6.09623 | 0.509488 | 2% |
| B1157 | 0.275696 | 1.687899 | 6.499076 | 5.868894 | 12.11262 | 6.805742 | 4% |
| B1186 | 0.411365 | 2.05024 | 8.10102 | 21.44188 | 18.84054 | 10.52496 | 0% |
| B1062 | 0.42324 | 0.462708 | 0.939028 | 4.059568 | 7.088249 | 0.528961 | 18% |
| B1174 | 0.139464 | 0.013101 | 0.02865 | 4.605733 | 5.128944 | 0.436957 | 0% |
| B1179 | 0.035694 | 0.012051 | 0.463515 | 5.568106 | 7.187314 | 0.978635 | 5% |
| B1185 | 0.309813 | 0.04515 | 0.437537 | 14.40698 | 39.26787 | 2.530794 | 0% |
| B1176 | 0.121472 | 0.624136 | 2.15097 | 3.24173 | 6.452212 | 3.382903 | 3% |
| B1115 | 0.136667 | 0.085789 | 0.161923 | 3.129635 | 3.826509 | 0.412866 | 20% |
| B1151 | 0.391941 | 0.468229 | 0.520322 | 4.469729 | 3.102957 | 0.682666 | 30% |
| B1130 | 0.0399 | 0.065142 | 0.141022 | 2.047363 | 1.865712 | 0.360232 | 5% |
| B1140 | 0.054809 | 0.193735 | 0.279461 | 1.414274 | 1.242459 | 0.122068 | 49% |
| B1162 | 0.845385 | 2.514363 | 5.297522 | 6.479206 | 11.88172 | 7.303862 | 0% |
| B1158 | 0.194648 | 0.021575 | 0.002181 | 5.301383 | 4.570718 | 0.867642 | 8% |
| B1165 | 0.026556 | 0.009037 | 0.142758 | 1.100014 | 1.915991 | 0.670915 | 4% |
| B1170 | 0.027755 | 0.002945 | 0.085162 | 2.402569 | 0.951581 | 0.133502 | 11% |
| B1166 | 0.016785 | 0.007146 | 0.116701 | 0.685067 | 1.738388 | 0.222897 | 12% |
| B1169 | 0.551674 | 1.122881 | 7.158117 | 6.25612 | 15.81405 | 1.86794 | 100% |
| B1181 | 0.158415 | 0.669712 | 2.341749 | 2.442243 | 7.922128 | 4.335333 | 1% |
| B1156 | 0.385409 | 2.436252 | 10.60516 | 11.90949 | 18.02418 | 11.3668 | 0% |
| B1077 | 0.186693 | 0.554774 | 1.924883 | 3.828849 | 6.595334 | 0.548293 | 1% |
| B1183 | 0.077487 | 0.013783 | 0.066009 | 2.989644 | 2.486809 | 0.435026 | 14% |
| B1171 | 0.121519 | 0.651149 | 1.6993 | 4.134024 | 6.984474 | 3.987267 | 1% |
| B1173 | 0.321653 | 0.590683 | 7.989787 | 8.788349 | 13.53742 | 1.043001 | 95% |
| B1177 | 0.389519 | 1.618576 | 6.919049 | 6.363478 | 12.67268 | 7.254963 | 0% |
| B1175 | 0.309556 | 1.024369 | 6.99008 | 8.446225 | 0.269706 | 7.198733 | 0% |
| B1144 | 0.124363 | 0.358677 | 2.757126 | 6.353329 | 8.419335 | 0.767167 | 2% |
| B1091 | 0.138649 | 0.488198 | 0.488367 | 2.316347 | 0.273047 | 1.89974 | 9% |
| B1119 | 0.086862 | 0.143832 | 0.310711 | 1.881186 | 0.504469 | 3.914304 | 0% |
| B1093 | 0.47004 | 0.651684 | 1.988406 | 3.888647 | 0.664054 | 5.681559 | 0% |
| B1090 | 0.228293 | 0.46156 | 0.434122 | 3.131433 | 0.606167 | 6.921811 | 0% |
| B1086 | 0.110698 | 0.333536 | 4.234953 | 9.227758 | 19.17408 | 3.971247 | 11% |
| B1088 | 0.08022 | 0.060692 | 0.439678 | 2.390222 | 2.880489 | 1.58442 | 3% |
| B1080 | 0.234844 | 0.075704 | 0.560799 | 2.414828 | 4.349285 | 1.876422 | 2% |
| B1069 | 0.058175 | 0.267825 | 0.6692 | 2.444818 | 3.897105 | 1.62294 | 17% |
| B1167 | 0.256362 | 1.545363 | 6.605418 | 7.087196 | 14.9399 | 7.923752 | 0% |
| B1146 | 0.304291 | 0.213407 | 0.650135 | 1.252683 | 1.823005 | 1.132733 | 8% |
| B1148 | 0.59862 | 3.071946 | 0.324258 | 1.945204 | 14.67802 | 9.625597 | 0% |
| B1128 | 0.203423 | 0.019272 | 0.09785 | 0.269751 | 0.773724 | 0.458157 | 8% |
| | 0.495427 | 0.389433 | 0.544696 | 4.151956 | 4.982279 | 0.594993 | 57% |
| B1098 | 0.511561 | 1.556267 | 5.473809 | 6.111935 | 15.25576 | 1.128555 | 100% |
| B1097 | 0.011705 | 0.028368 | 0.151111 | 0.59118 | 0.401502 | 0.033452 | 48% |
| B1099 | 0.29597 | 1.194941 | 0.120174 | 5.332556 | 13.75178 | 1.712032 | 0% |
| B1150 | 0.075059 | 0.301157 | 0.656832 | 1.017529 | 2.758191 | 0.184487 | 60% |
| B1113 | 0.134593 | 0.155909 | 1.356737 | 4.632578 | 1.384212 | 4.812153 | 0% |
| B1109 | 3.321575 | 0.520585 | 6.241235 | 31.49144 | 2.328687 | 3.130623 | 4% |
| B1096 | 0.07738 | 0.343346 | 1.814182 | 1.955658 | 5.553138 | 0.555758 | 76% |
| B1123 | 0.145683 | 0.482514 | 0.949824 | 1.702649 | 2.869734 | 0.282746 | 81% |
| B1104 | 0.806964 | 1.57114 | 14.61101 | 12.89248 | 17.89394 | 1.686985 | 100% |
| B1184 | 0.221308 | 2.254178 | 5.506958 | 4.54257 | 3.548859 | 0.833887 | 100% |
| B1205 | 0.106993 | 0.934333 | 6.083491 | 2.522991 | 3.017094 | 0.621039 | 100% |
| B1196 | 0.210903 | 1.016147 | 5.027299 | 2.150913 | 3.617493 | 0.466242 | 100% |
| B1225 | 0.113026 | 0.352238 | 3.054674 | 2.703137 | 3.470859 | 0.275564 | 95% |
| B1212 | 0.059815 | 0.30924 | 5.644955 | 2.360243 | 0.834177 | 1.401678 | 98% |
| B1214 | 0.17825 | 1.780498 | 9.633003 | 5.229102 | 6.850583 | 1.122241 | 100% |
| B1198 | 0.388126 | 2.435847 | 8.054816 | 5.488663 | 5.151745 | 0.738789 | 100% |
| B1238 | 0.357251 | 0.973025 | 10.29985 | 4.027003 | 4.412272 | 0.936932 | 100% |
| B1208 | 0.314877 | 1.129433 | 7.674666 | 7.07746 | 10.87933 | 1.2411 | 98% |
| B1141 | 0.059981 | 0.21044 | 4.222828 | 1.677524 | 1.573974 | 0.17079 | 99% |
| B1227 | 0.130221 | 0.598613 | 9.607954 | 1.509017 | 3.030802 | 0.578554 | 100% |
| B1228 | 0.104252 | 0.434244 | 2.17247 | 0.59609 | 0.842983 | 0.183714 | 95% |
| B1216 | 0.117852 | 0.845669 | 7.460972 | 1.470069 | 2.175548 | 0.314144 | 100% |
| | 0.229512 | 0.653096 | 2.905174 | 4.660052 | 6.516955 | 2.609535 | |
| | 0.213717 | 0.931362 | 5.047529 | 3.918729 | 4.892015 | 0.752267 | |
| | 0.230873 | 0.611085 | 1.400325 | 3.187506 | 5.380279 | 3.88439 | |
| | 0.207689 | 0.599181 | 2.132477 | 4.633784 | 6.942662 | 2.596213 | |
| | 0.874121 | 0.025039 | 4.74E−05 | 0.256176 | 0.057363 | 8.65E−05 | |
| | MAPK9 | NKTR | NAMPT | PSEN1 | RNF130 | RYBP | |
| | −0.0158 | 0.278266 | 2.142354 | −0.74132 | −1.62494 | −1.85727 | |

### Diagnostic Capability of 5-Gene Model

The logistic regression model selected is shown below, where 0 is the predicted probability for a sample to be classified as AR.

$$θ = \frac{{e}^{0.27 + ( - 0.13 * DUSP 1 ) + ( - 0.2 * IFNGR 1 ) + ( 2.96 * MAPK 9 ) + ( 1.4 \; 6 * PBEF 1 ) + ( - 1.58 * RYBP )}}{\begin{matrix}1 \div \\ {e}^{0.27 + ( - 0.13 * DUSP 1 ) + ( - 0.2 * IFNGR 1 ) + ( 2.96 * MAPK 9 ) + ( 1.4 \; 6 * PBEF 1 ) + ( - 1.58 * RYBP )}\end{matrix}}$$

Based on the Receiver Operating Characteristic (ROC) curve, a cutoff of θ=0.37 was selected to have the best sensitivity and specificity to discriminate between AR and STA. In this model, each of the regression coefficients describes the size of the contribution of that gene as a risk factor for diagnosing AR, where the larger the coefficient, the greater the influence of that gene in AR. A positive coefficient suggests that the explanatory variable increases the probability of AR, where a negative coefficient decreases the probability of AR.

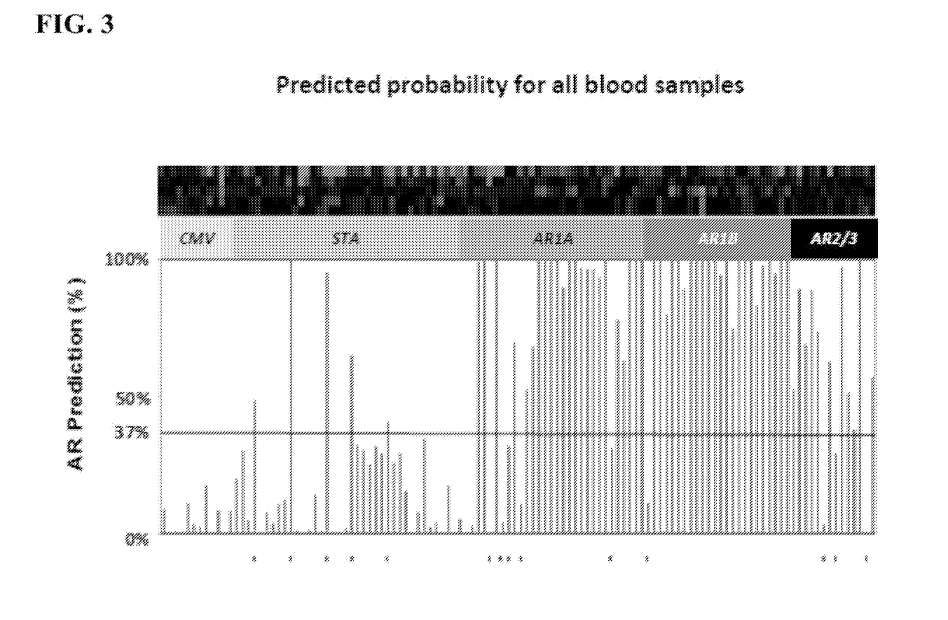

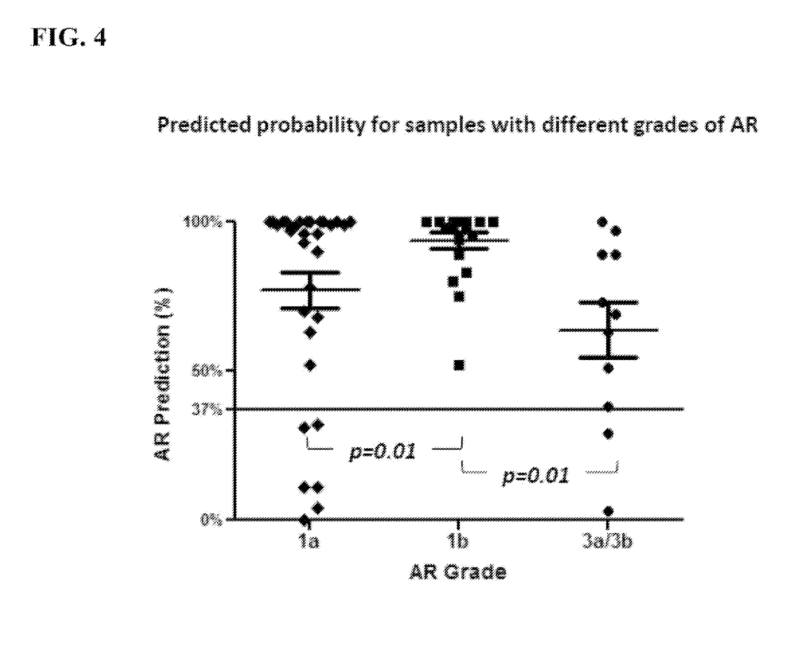

A threshold 0 of 0.37 was selected for the best sensitivity and specificity, based on the Receiver Operating Characteristic (ROC) curve with an AUC of 0.89, to determine whether the predictive class was AR or STA (the asterisk shows the samples in each class that were misclassified; FIG. 3). The 5-gene set was subsequently tested in 86 independent samples and identified the AR phenotype with 88% accuracy (with misclassification of 6 AR grade 1A and 3 STA samples; FIG. 3, Table 5). For the 86 samples in the prediction set, the overall sensitivity was 87%, specificity was 90%, PPV is 94%, and NPV was 80%. The individual prediction scores for the different AR grades are shown in Table 5. The sensitivity for prediction of acute rejection was highest for acute rejection of Grade 1B (100%), and was 82% for prediction of Grades 3A/B and 81% for Grade 1A. Sensitivity for prediction of Grade 2 events was not calculated as there were only 2 samples in this category and both classified correctly. The 5-gene prediction score could not segregate tissue samples that had fibrosis (Grade 3B; p=0.21) and myocyte damage (Grades 3A and 3B; p=0.07) from those with lesser grades of AR (Grades ≦2). As the prediction probability of detecting Grade 1B rejection in the blood sample was the highest, it is possible that the signal for the blood gene expression profile reflects the extent of the inflammatory response in the graft, which is greatest in acute rejections of Grade 1B (there was statistically significantly better prediction of Grade 1B vs Grade 1A; p=0.01; Grade 1B vs Grade 3A/B, p=0.01) (FIG. 4).

TABLE 5
Prediction Performance of the 5-Gene Model on Different
Clinical Phenotypes (Biopsy Confirmed)

| | AR | STA | | |
|---|---|---|---|---|
| Prediction | (predic- | (predic- | | Sensitivity (AR) or |
| Sets | tion) | tion) | Total | Specificity (Non-AR) |
| AR (N = 55) | 49 | 6 | 55 | 89% Sensitivity |
| 1A (N = 31) | 25 | 5 | 31 | 81% Sensitivity |
| 1B (N = 22) | 22 | 0 | 22 | 100% Sensitivity |
| 2 (N = 2) | 1 | 1 | 2 | Not calculated |
| Non-AR (N = 31) | 3 | 28 | 31 | 90% Specificity |
| STA (N = 19) | 3 | 16 | 19 | 84% Specificity |
| CMV+ (N = 12) | 0 | 12 | 12 | 100% Specificity |

AR: acute rejection (Grades 1-3);
STA: stable (Grade 0)

### Evaluation of Confounders Effects

To determine if demographic or clinical variables could be confounders of the chosen 5-gene model, Pearson correlation coefficients, T tests, and chi-square tests were used, as appropriate, to evaluate the association of 16 variables with the presence or absence of cellular rejection on biopsy (Table 5). The 16 variables included white blood cells (WBC), neutrophils (NEUT), lymphocytes (LYM), monocytes (MONO), eosinophils (EOS), basophils (BASO), sample time, recipient age at transplantation, recipient age at sample time, gender of recipient, and donor blood. These analyses did not identify any significant confounders (maximum |r|<0.4 or p>0.05), and specifically time-post transplant for sampling did not confound the score, which has been an issue in other biomarker studies of this nature. See Deng et al., Am J Transplant, 2006, 6(1):150-60. All CMV-positive samples were predicted correctly to have no acute rejection, suggesting that there is no concern for innate immune activation in CMV confounding the blood gene expression panel for acute rejection.

TABLE 6
Analysis of patient demographic variables

| r² | wbc % | NEUT % | LYM % | MONO % | EOS % | BASO % | NEUT_abs | LYM_abs | MONO_abs |
|---|---|---|---|---|---|---|---|---|---|
| DUSP1 | 0.046 | 0.226 | −0.239 | −0.143 | −0.036 | −0.187 | 0.078 | −0.102 | −0.107 |
| IFNGR1 | 0.137 | 0.098 | −0.113 | −0.043 | −0.085 | −0.253 | 0.120 | 0.012 | 0.040 |
| MAPK9 | 0.025 | −0.263 | 0.200 | 0.109 | 0.302 | −0.075 | −0.118 | 0.325 | 0.135 |
| NAMPT | 0.128 | 0.136 | −0.099 | −0.164 | −0.040 | −0.203 | 0.166 | −0.006 | −0.102 |
| RYBP | −0.090 | 0.171 | −0.118 | −0.173 | −0.211 | −0.255 | −0.065 | −0.112 | −0.213 |

| r² | EOS_abs | BASO_abs | sample_time | age_txp | age_sample | gender | donor_BLD |
|---|---|---|---|---|---|---|---|
| DUSP1 | 0.043 | 0.007 | −0.035 | −0.057 | −0.060 | 0.63 | 0.34 |
| IFNGR1 | 0.022 | −0.099 | −0.035 | −0.030 | −0.032 | 0.95 | 0.16 |
| MAPK9 | 0.371 | −0.042 | 0.327 | −0.174 | −0.159 | 0.55 | 0.61 |
| NAMPT | 0.031 | −0.130 | −0.099 | 0.021 | 0.015 | 0.9 | 0.76 |
| RYBP | −0.147 | −0.201 | −0.086 | 0.116 | 0.113 | 0.33 | 0.88 |

### Prediction of Acute Cellular Rejection Prior to Diagnosis by Endomyocardial Biopsy

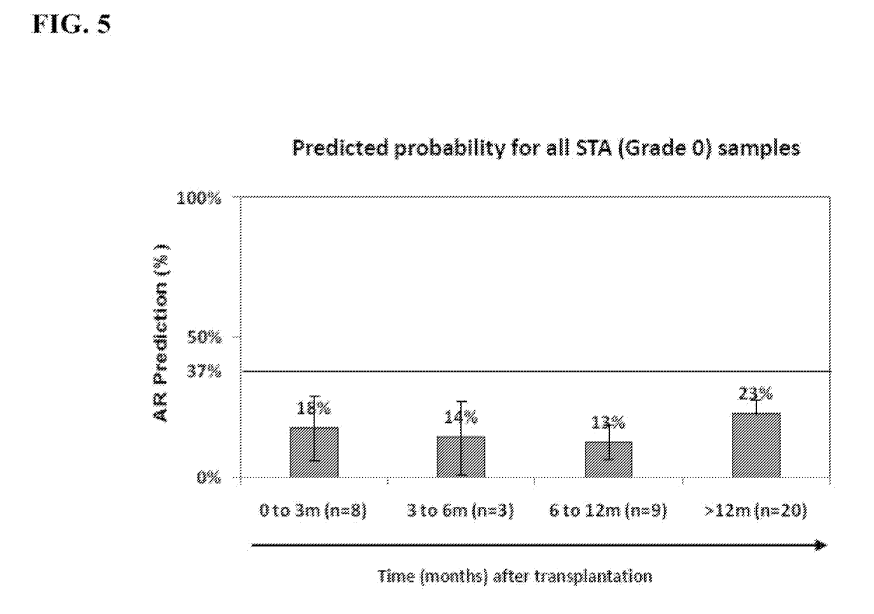

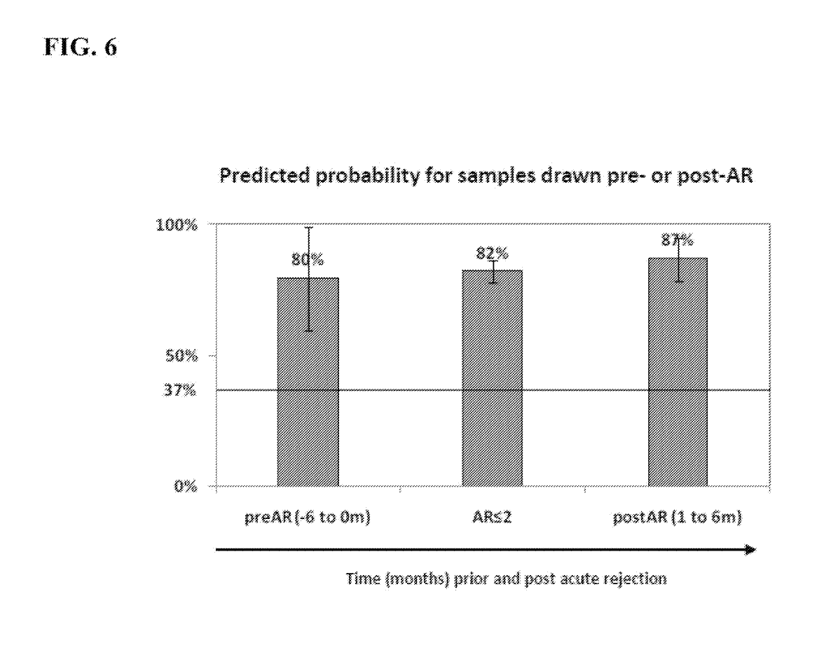

The 5-gene model was examined for its ability to predict acute rejection from a blood sample drawn within 1-6 months prior to the biopsy proven acute rejection event. This analysis was done to evaluate if there was a greater chance of predicting an upcoming AR episode, prior to its detection by biopsy. The prediction score from blood samples drawn within a period of 6 months prior to a biopsy proven AR event (grades 1A, 1B, or 2) or absence of acute rejection was assessed (FIG. 1). There was a statistically higher likelihood (p<0.0001) of a high prediction score for AR (mean prediction score 80%; FIG. 6) in the blood samples drawn prior to an acute rejection episode than a blood sample drawn prior to a negative biopsy (mean prediction score 17%; FIG. 5). The 5-gene probability score for acute rejection in many blood samples drawn within 1-6 months after treatment of acute rejection varied between (0%-100%), with an average prediction score of 87% (n=12 samples; FIG. 6).

Acute Rejection Prediction Score is Significantly Associated with Development of Cardiac Allograft Vasculopathy

There was a significant positive correlation between the probability score for prediction of AR in a blood sample drawn at 1 year post-transplantation, and the subsequent development of CAV in that same patient at 2 years (r=0.73, p=0.02) and at 4 years (r=0.82, p=0.01) post-transplantation. Furthermore, predicted probabilities of AR at 1 year were significantly higher in patients with higher grades of CAV (CAV score≧3) vs. mild grades of CAV (CAV score ≦2) at 4 years post-transplantation (99%±1% vs. 32%±14%, p=0.001), which indicate that patients with higher predicted AR probability at the early follow-up may be at greater risk to develop more severe CAV at subsequent follow-up.

Donor Derived Cell-Free DNA (cfDNA) is a Marker of Transplant Injury Burden

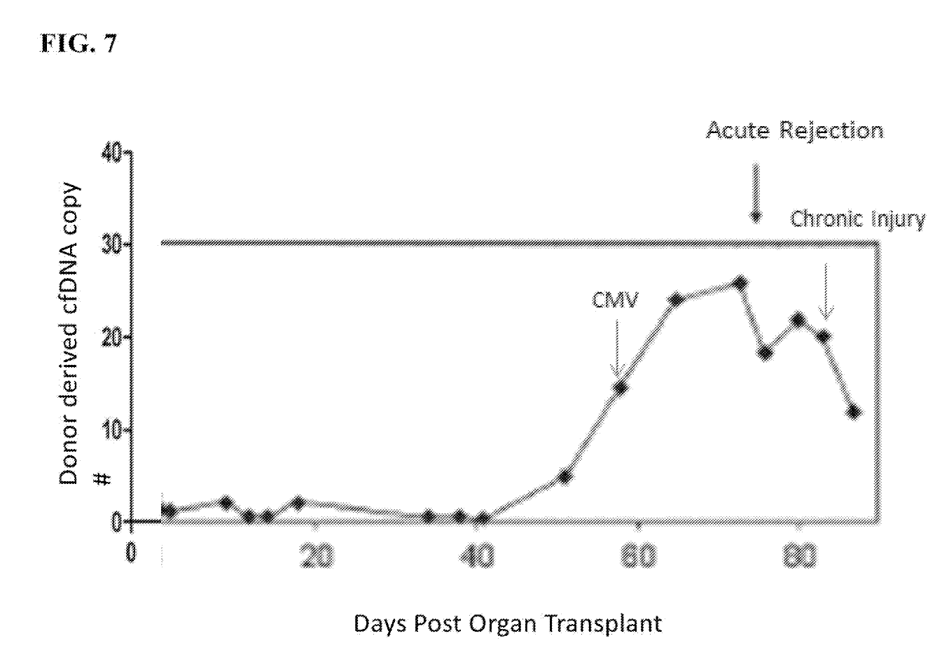

Chromosomal copy number was determined from patients at different time points post-transplantation. Increases in donor derived cell-free DNA was detected months before actual organ graft injury. Further increases in donor derived cell-free DNA was observed following different types of injury corresponding to cytomegalovirus (CMV) infection, acute rejection, or chronic injury with each type of donor organ injury corresponding to a different chromosomal copy number (FIG. 7).

Discussion

This is the first study to cross-validate a gene expression panel that detects acute rejection after kidney transplantation for detection and prediction of acute rejection in heart transplant recipients. The 10-gene panel is differentially regulated in the periphery at the time of histologically confirmed acute rejection irrespective of tissue source. Additionally these genes are indicative of histological acute rejection in both children and adults, as the kidney PCR data (see Li et al., Am J Transplant, 2012, 12(10):2710-8) was discovered and validated in pediatric and young adult renal allograft recipients and the heart PCR data in this paper has been validated in adult heart transplant recipients. Due to the tight correlation between individual genes in the panel, it was possible to narrow the original 10-gene panel to an even smaller set of 5-genes that is not confounded by clinical variables, such as transplant recipient age and sex, time post-transplant, or the presence of concomitant CMV infection. The lack of any confounding effect from active CMV infection suggests that the gene expression signature reflects the identification of a specific alloimmune trafficking response that is independent of the heightened innate immune response seen in CMV infection.

This peripheral blood gene expression signature correlates strongly with the activation profile of the inflammatory infiltrate, rather than the grade of rejection or the extent of fibrosis or myocyte damage. These genes have been shown to be highly expressed in cells of the monocyte and macrophage lineage (see Li et al., Am J Transplant, 2012, 12(10):2710-8; Bromberg et al., Am J Transplant, 2012, 12(10):2573-4), suggesting that the gene expression panel is detecting trafficking of activated monocyte lineage cells. These cells may be common to the inflammatory injury of acute rejection in kidney and heart transplantation. Other markers of immune activation and inflammation have been identified in blood and tissue as biomarkers of acute rejection. CD27, CD40, TIRC7, cytokines (interferon-γ, interleukin [IL]-2, IL-4, IL-6, IL-8), and cytotoxic T-cell effector molecules (perforin, granzyme B, FasL) have been found to be elevated in rejecting biopsy samples (see Alpert et al., Transplantation, 1995, 60(12):1478-85; Baan et al., Clin Exp Immunol., 1994, 97(2):293-8; de Groot-Kruseman et al., Heart, 2002, 87(4):363-7; Shulzhenko et al., Braz J Med Biol Res., 2001, 34(6):779-84; Shulzhenko et al., Hum Immunol., 2001, 62(4):342-7; Shulzhenko et al., Transplantation, 2001, 72(10):1705-8; van Emmerik et al., Transpl Int., 1994, 7 Suppl 1:S623-6) and peripheral in blood (see Kimball et al., Transplantation, 1996, 61(6):909-15; Lagoo et al., J Heart Lung Transplant, 1996, 15(2):206-17; Morgun et al., Transplant Proc., 2001, 33(1-2):1610-1) at the time of cardiac allograft rejection. Microarray technologies offer the option of simultaneously screening thousands of novel candidate genes in an unbiased fashion, while controlling for multiple clinical confounders, enabling the identification of panels of genes in peripheral blood that may be very sensitive and specific for histological acute rejection (see Sarwal et al., N Engl J Med., 2003, 349(2):125-38; Khatri et al., Curr Opin Organ Transplant, 2009, 14(1):34-9) and provide more robust performance than any single gene analysis (see Deng et al., Am J Transplant, 2006, 6(1):150-60; Horwitz et al., Circulation, 2004, 110(25):3815-21).

The discovery of the robust 10 gene-set in this study came from global gene expression analysis of ˜54,000 genes on different microarray platforms using peripheral blood samples from pediatric kidney transplant recipients (see Ying et al., American Journal of Transplantation, 2008, 8(S2):248) was validated in a prospective, randomized multicenter clinical trial. The same biomarkers can detect AR in adult heart transplant recipients, which highlights the power of this gene-set to detect biopsy confirmed AR, not only in different solid organs but also across the span of gender, post-transplant time, differences in immunosuppression, transplant centers and recipient age. The Cardiac Allograft Rejection Gene expression Observational (CARGO) study (see Deng et al., Am J Transplant, 2006, 6(1):150-60), identified an 11-gene PCR classifier, largely from the literature, that was subsequently commercialized into the AlloMap Molecular Expression Test (XDx, Brisbane, Calif.). This test provides a negative predictive value (NPV) of 99% for moderate-severe cellular rejection by EMB, providing a means for ruling-out the presence of rejection but has low positive predictive value and sensitivity for detection of AR. The clinical utility of a blood gene profiling approach for ruling out acute rejection was subsequently demonstrated in a randomized study on 600 heart transplant recipients, where there was non-inferiority of an Allomap-based rejection monitoring strategy, compared to EMB, with respect to a composite endpoint of acute rejection, graft failure and death, and a reduction in the number of EMBs performed in this study by almost 70%, consistent with the high negative predictive value associated with the Allomap test (see Pham et al., N Engl J Med., 362(20):1890-900). However, the positive predictive value of 20-40% for the Allomap test for detecting the presence of acute rejection suggests that complementary approaches for the diagnosis and prediction of acute rejection, such as the use of the gene-panel in this study, are needed.

Although management of heart transplant recipients often varies between centers, most transplant programs only consider rejection of Grade 3A or 3B (showing myocyte damage) as clinically relevant, and therefore warranting treatment. Currently, acute rejection of grades of 1A, 1B and 2 are frequently dismissed, without any additional treatment delivery, perhaps because these lower histological grades of rejection are observed so commonly in the protocol biopsies performed. The inflammatory infiltrate that is common to all histological grades (1-4) of acute rejection and is singularly absent in the non-rejection biopsies (Grade 0), suggests that the presence of an infiltrate is a very common finding, and in the absence of myocyte damage its clinical relevance in heart transplantation remains unclear. Nevertheless, the presence of an inflammatory infiltrate of predominantly mononuclear cells is the hallmark of acute rejection in other solid organ transplants such as kidney (see Solez et al., Kidney Int., 1993, 44(2):411-22), lung (see Stewart et al., J Heart Lung Transplant, 2007, 26(12):1229-42) and small intestine (see Wu et al., Transplantation, 2003, 75(8):1241-8), where the infiltrate is believed to be pathologically and clinically relevant, and triggers a treatment response of bolus immunosuppression. The ISHLT 1990 classification scheme for acute cardiac allograft rejection distinguished 3 grades of mild-moderate cellular rejection: Grades 1A, 1B, and 2, based on absence (Grades 1A and 1B) or presence of myocyte damage (Grade 2), and focal (Grade 1A) versus diffuse (Grade 1B) nature of the lymphocytic infiltrate (Table 2). Subsequent clinical investigations of these mild-moderate rejection grades focused on their temporal occurrence, requirement for therapy, and progression to more severe grades of rejection, (see Delgado et al., Clin Transplant, 2002, 16(3):217-21; Fishbein et al., J Heart Lung Transplant, 1994, 13(6):1051-7; Nielsen et al., J Heart Lung Transplant, 1993, 12(2):239-43; Winters et al., J Heart Lung Transplant, 1996, 15(7):728-35; Yeoh et al., Circulation, 1992, 86(5 Suppl):II267-71) and ultimately led to a revision of the ISHLT classification scheme in 2004, which included a single mild grade of rejection (1R), which subsumed the original Grades 1A, 1B, and 2 (see Stewart et al., J Heart Lung Transplant, 2005, 24(11):1710-20).

The 5-gene model tested in this study can diagnose acute rejection of Grades 1A-3B (no Grade 4 samples were available for this study), with the highest confidence for diagnosing Grade 1B rejection. Molecular subtyping has demonstrated evidence of myocyte apoptosis in Grade 1B biopsies that is a feature of myocyte damage typical of Grade 3A biopsies, but not of less severe (Grade 1A) rejection (see Laguens et al., J Heart Lung Transplant, 1996, 15(9):911-8). Such data suggests that Grade 1B biopsies may share molecular similarities with Grades ≧3A, and that molecular approaches may provide novel insights into tissue injury that may complement the light-microscopic criteria traditionally used for biopsy grading. Bernstein et al (see Bernstein et al., J Heart Lung Transplant, 2007, 26(12):1270-80) recently performed a post hoc analysis of the CARGO data, specifically examining gene expression scores for blood samples accompanying endomyocardial biopsies of varying grades. They demonstrated that the mean gene expression scores for Grades 1B and ≧3A were indistinguishable, once again suggesting their potential overlap along a molecular spectrum of rejection severity. A recent study by Holweg et al. (see Holweg et al., Circulation, 2011, 123(20):2236-43) profiled endomyocardial biopsies of patients with different cardiac transplant rejection grades. Although grade 1B was found to be distinct from the clinically relevant AR grades 3A and 3B, all of these grades were found to share a number of overlapping pathways consistent with common physiological underpinnings. The mean gene expression score for Grade 1B also suggests its molecular distinction from other Grades (1A and 2) classified as mild rejection in the 2004 revised grading scheme (see Stewart et al., J Heart Lung Transplant, 2005, 24(11):1710-20). The results herein are consistent with those of Bernstein, and suggest that combining Grades 1A, 1B, and 2 in the 2004 revised grading scheme may undermine the independent value and distinct inflammatory nature of different rejection grades. The gene expression similarities identified here in grade 1B and grade 3 AR have the potential to revise the clinical perspective on acute graft rejection, pending the results of additional prospective studies.

The 5-gene model developed in this study can also predict the onset of acute rejection, months before it is diagnosed by protocol biopsy. Importantly, the score decreases after augmented immunosuppressive therapy in patients with rejection grades 3A/B, and remains elevated in untreated cases of acute rejection of grades ≦2.

Recent work in kidney transplantation (see Li et al., Am J Transplant, 2012, 12(10):2710-8; Sarwal et al., N Engl J Med., 2003, 349(2):125-38; Ying et al., American Journal of Transplantation, 2008; 8(S2):248; Shen-Orr et al., Nat Methods, 7(4):287-9) has highlighted the fact that the 10 selected genes in the original model are highly expressed in cells of the monocyte lineage. The statistical approach of deconvolution (see Shen-Orr et al., Nat Methods, 7(4):287-9), now available as cell-specific Significance Analysis of Microarrays or cSAM (see Tusher et al., Proc Natl Acad Sci USA, 2001, 98(9):5116-21), also demonstrates that the monocyte-specific signal in peripheral blood (see Li et al., Am J Transplant, 2012, 12(10):2710-8; Bromberg et al., Am J Transplant, 2012, 12(10):2573-4) drives the differential expression of peripheral genes in acute renal transplant rejection. As the previous studies in kidney transplant rejection (see Shen-Orr et al., Nat Methods, 7(4):287-9) have not identified any differences in the numbers of circulating monocytes, the gene signature likely reflects an activation status of this cell lineage. As this same gene set also displays differential regulation in all grades of acute heart transplant rejection, this work highlights a novel, and hitherto unrecognized role for the activated monocyte as the key peripheral trafficking cell in acute rejection, both within the graft and as a biomarker for acute rejection in the periphery.

CAV, the leading cause of late morbidity and mortality after heart transplantation, is a complex multifactorial process mediated by both immune and non-immune factors. The diffuse nature of CAV, which usually involves the entire coronary arterial tree (see Russell et al., Transplantation, 1993, 56(6):1599-601) suggests primarily an immune etiology. Prior observational studies suggest that cellular AR and CAV are closely related processes (see Stoica et al., J Heart Lung Transplant, 2006, 25(4):420-5; Hornick et al., Circulation, 1997, 96(9 Suppl):II-148-53). The finding of a positive association between AR prediction scores and subsequent development of CAV further supports this theory. A similar finding was also noted by the an association of the AlloMap with cardiac vasculopathy, as a higher AlloMap score was found in 20 cardiac recipients with EMB confirmed vasculopathy and compared to 49 control patients (see Yamani et al., J Heart Lung Transplant, 2007, 26(4):403-6). Thus the finding herein also supports that gene expression testing could be used to determine a patient's future risk of CAV—and to potentially tailor prophylactic strategies to prevent CAV development. The strong correlation seen for the AR prediction score of the current 5-gene model with the development of subsequent CAV suggests that this inflammatory infiltrate, even independent of rejection grade and similar to its downstream effect in other solid organs (see Horwitz et al., Circulation, 2004, 110(25):3815-21; Ying et al., American Journal of Transplantation, 2008, 8(S2):248; Pham et al., N Engl J Med., 2010, 362(20):1890-900), may not be benign and likely accelerates the evolution of chronic injury, and is therefore potentially deserving of clinical vigilance and treatment.

In conclusion, an internally validated 5-gene classifier panel, from a larger set of 10 genes, has been developed to non-invasively screen for the presence of acute cellular rejection after heart transplantation. The high specificity and positive predictive value of the 5-gene panel in peripheral blood samples fulfills a critical unmet need for acute rejection monitoring in heart transplantation. As mentioned above, the currently-available AlloMap test has very high negative predictive value, and therefore enables clinicians to rule out the presence of rejection. This assay, with a high positive predictive value, would therefore be complementary by concurrently enabling clinicians to rule in the presence of rejection and can additionally predict a risk-read out for acute rejection prior to any clinical graft dysfunction. A strategy that combines both non-invasive tests could therefore enable biopsy avoidance in a larger number of patients than either test alone. The observed gene expression patterns in this study challenge the current paradigm of classifying certain rejection grades, such as Grade 1B, as “mild” and therefore not requiring intensification of immunosuppressive therapy.

### Example 2

### Diagnosis and Prediction of Acute Rejection of Lung Transplant

Similar to the study described in Example 1, correlation studies of gene expression profiles in 10 peripheral blood samples of lung transplant patients with biopsy-proven acute rejection as compared to 10 peripheral blood samples of lung transplant patients without acute rejection results in the identification of all 10 genes (i.e., CFLAR, DUSP1, IFNGR1, ITGAX, NAMPT, PSEN1, RNF130, RYBP, MAPK9, and NKTR). Differential expression analysis is further conducted in bronchoalveolar lavage (BAL) samples and further confirms the differential gene expression for the 10 genes.

### Example 3

### Diagnosis and Prediction of Acute Rejection of Liver Transplant

A similar study as described in Example 1 is done with subjects who have received a liver transplant. Correlation studies of gene expression profiles in 15 peripheral blood samples of liver transplant patients with biopsy-proven acute rejection as compared to 45 peripheral blood samples of liver transplant patients without acute rejection results in the identification of all 10 genes (i.e., CFLAR, DUSP1, IFNGR1, ITGAX, NAMPT, PSEN1, RNF130, RYBP, MAPK9, and NKTR).

### Example 4

### Diagnosis and Prediction of Acute Rejection of Intestinal Transplants

Similar to the study described in Example 1, correlation studies of gene expression profiles in 5 peripheral blood samples of intestinal transplant patients with biopsy-proven acute rejection as compared to 5 peripheral blood samples of intestinal transplant patients without acute rejection results in the identification of all 10 genes (i.e., CFLAR, DUSP1, IFNGR1, ITGAX, NAMPT, PSEN1, RNF130, RYBP, MAPK9, and NKTR) to be significant for diagnosing and predicting acute rejection of intestinal transplant.

1. A method for aiding in the diagnosis of an acute rejection response in a subject who has received a solid organ allograft, the method comprising:
a) detecting a gene expression level for at least ten genes in a sample from the subject, wherein the at least ten genes comprise CFLAR, DUSP1, IFNGR1, ITGAX, MAPK9, NAMPT, NKTR, PSEN1, RNF130, and RYBP; and
b) comparing the gene expression level to a reference expression level of the at least ten genes, wherein a statistical difference or a statistical similarity between the gene expression level and the reference expression level of at least five genes selected from the group consisting of CFLAR, DUSP1, IFNGR1, ITGAX, MAPK9, NAMPT, NKTR, PSEN1, RNF130, and RYBP, thereby aiding in the diagnosis of an acute rejection response.

2. The method of claim 1, wherein the reference expression level is obtained from a control sample from at least one subject with an acute rejection response to a solid organ allograft.

3. The method of claim 2, wherein the statistical similarity between the gene expression level and the reference expression level for the at least five genes aids in the diagnosis of an acute rejection response in the subject.

4. The method of claim 2, wherein the statistical difference between the gene expression level and the reference expression level for the at least five genes aids in the diagnosis of the absence of an acute rejection response in the subject.

5. The method of claim 1, wherein the reference expression level is obtained from a control sample from at least one subject without an acute rejection response to a solid organ allograft.

6. The method of claim 5, wherein the statistical similarity between the gene expression level and the reference expression level for the at least five genes aids in the diagnosis of the absence of an acute rejection response in the subject.

7. The method of claim 5, wherein the statistical difference between the gene expression level and the reference expression level for the at least five genes aids in the diagnosis of an acute rejection response in the subject.

8. The method as in any one of claims 1-7, wherein the sample is a biological sample.

9. The method of claim 8, wherein the biological sample is selected from the group consisting of: a blood sample, a biopsy sample, a saliva sample, a cerebrospinal fluid sample, or a urine sample.

10. The method of claim 8, wherein the biological sample comprises peripheral blood leukocytes.

11. The method of claim 8, wherein the biological sample comprises peripheral blood mononuclear cells.

12. The method of claim 8, wherein the biological sample is a bronchoalveolar lavage sample.

13. The method as in any one of claims 1-12, wherein the solid organ allograft is one or more selected from the group consisting of: heart, lung, large intestine, small intestine, liver, kidney, pancreas, stomach, and bladder.

14. The method as in any one of claims 1-13, wherein the step of detecting comprises assaying the sample for an expression product of the at least ten genes.

15. The method of claim 14, wherein the expression product is a nucleic acid transcript.

16. The method of claim 14, wherein the expression product is a protein.

17. The method as in any one of claims 1-13, wherein the step of detecting comprises assaying the expression of the at least ten genes by hybridizing nucleic acids to oligonucleotide probes, by RT-PCR or by direct mRNA capture.

18. The method as in any one of claims 1-13, wherein the step of detecting comprises assaying the expression of the at least ten genes on one or more of: an array, a bead, and a nanoparticle.

19. The method as in any one of claims 1-18, wherein the subject has a cardiac acute rejection score of Grade 0, Grade 1A, Grade 1B, Grade 2, Grade 3A, Grade 3B, or Grade 4.

20. The method as in any one of claims 1-19, wherein the comparing step aids in the diagnosis of acute rejection with equal to or greater than 70% sensitivity.

21. The method as in any one of claims 1-20, wherein the comparing step aids in the diagnosis of acute rejection with equal to or greater than 70% specificity.

22. The method as in any one of claims 1-21, wherein the comparing step aids in the diagnosis of acute rejection with equal to or greater than 70% positive predictive value (ppv).

23. The method as in any one of claims 1-22, wherein the comparing step aids in the diagnosis of acute rejection with equal to or greater than 70% negative predictive value (npv).

24. A method for predicting the likelihood of an acute rejection response in a subject who has received a solid organ allograft, the method comprising:
a) detecting a gene expression level for at least ten genes in a sample from the subject, wherein the at least ten genes comprise CFLAR, DUSP1, IFNGR1, ITGAX, MAPK9, NAMPT, NKTR, PSEN1, RNF130, and RYBP; and
b) comparing the gene expression level to a reference expression level of the at least ten genes,
wherein a statistical difference or a statistical similarity between the gene expression level and the reference expression level for at least five genes selected from the group consisting of CFLAR, DUSP1, IFNGR1, ITGAX, MAPK9, NAMPT, NKTR, PSEN1, RNF130, and RYBP, thereby predicting the likelihood of an acute rejection response in the subject.

25. The method of claim 24, wherein the reference expression level is obtained from a control sample from at least one subject with an acute rejection response to a solid organ allograft.

26. The method of claim 25, wherein the statistical similarity between the gene expression level and the reference expression level for the at least five genes predicts the likelihood of an acute rejection response in the subject.

27. The method of claim 25, wherein the statistical difference between the gene expression level and the reference expression level for the at least five genes predicts the likelihood of the absence of an acute rejection response in the subject.

28. The method of claim 24, wherein the reference expression level is obtained from a control sample from at least one subject without an acute rejection response to a solid organ allograft.

29. The method of claim 28, wherein the statistical similarity between the gene expression level and the reference expression level for the at least five genes predicts the likelihood of the absence of an acute rejection response in the subject.

30. The method of claim 28, wherein the statistical difference between the gene expression level and the reference expression level for the at least five genes predicts the likelihood of an acute rejection response in the subject.

31. The method as in any one of claims 24-30, wherein the sample is a biological sample.

32. The method of claim 31, wherein the biological sample is selected from the group consisting of: a blood sample, a biopsy sample, a saliva sample, a cerebrospinal fluid sample, or a urine sample.

33. The method of claim 31, wherein the biological sample comprises peripheral blood leukocytes.

34. The method of claim 31, wherein the biological sample comprises peripheral blood mononuclear cells.

35. The method of claim 31, wherein the biological sample is a bronchoalveolar lavage sample.

36. The method as in any one of claims 24-35, wherein the solid organ allograft is one or more selected from the group consisting of: heart, lung, large intestine, small intestine, liver, kidney, pancreas, stomach, and bladder.

37. The method as in any one of claims 24-36, wherein the step of detecting comprises assaying the sample for an expression product of the at least ten genes.

38. The method of claim 37, wherein the expression product is a nucleic acid transcript.

39. The method of claim 37, wherein the expression product is a protein.

40. The method as in any one of claims 24-36, wherein the step of detecting comprises assaying the expression of the at least ten genes by hybridizing nucleic acids to oligonucleotide probes, by RT-PCR or by direct mRNA capture.

41. The method as in any one of claims 24-36, wherein the step of detecting comprises assaying the expression of the at least ten genes on one or more of: an array, a bead, and a nanoparticle.

42. The method as in any one of claims 24-41, wherein the subject has a cardiac acute rejection score of Grade 0, Grade 1A, Grade 1B, Grade 2, Grade 3A, Grade 3B, or Grade 4.

43. The method as in any one of claims 24-42, wherein the comparing step predicts the likelihood of acute rejection response with equal to or greater than 70% sensitivity.

44. The method as in any one of claims 24-43, wherein the comparing step predicts the likelihood of acute rejection response with equal to or greater than 70% specificity.

45. The method as in any one of claims 24-44, wherein the comparing step predicts the likelihood of acute rejection response with equal to or greater than 70% positive predictive value (ppv).

46. The method as in any one of claims 24-45, wherein the comparing step predicts the likelihood of acute rejection response with equal to or greater than 70% negative predictive value (npv).

47. The method as in any one of claims 24-46, wherein the expression level of the at least five genes is employed to predict the likelihood of an acute rejection response within 1 to 6 months of obtaining the sample.

48. A method for monitoring the progression an acute rejection response in a subject who has received a solid organ allograft, the method comprising:
a) detecting a gene expression level for at least ten genes in a sample from the subject, wherein the at least ten genes comprise CFLAR, DUSP1, IFNGR1, ITGAX, MAPK9, NAMPT, NKTR, PSEN1, RNF130, and RYBP; and
b) comparing the gene expression level to a reference expression level of the at least ten genes; and
c) determining whether the subject has an acute rejection response based upon a statistical difference or a statistical similarity between the gene expression level and the reference expression level of at least five genes selected from the group consisting of CFLAR, DUSP1, IFNGR1, ITGAX, MAPK9, NAMPT, NKTR, PSEN1, RNF130, and RYBP, thereby monitoring the progression of an acute rejection response in the subject.

49. The method of claim 48, wherein the reference expression level is obtained from a control sample from at least one subject with an acute rejection response to a solid organ allograft.

50. The method of claim 49, wherein the statistical similarity between the gene expression level and the reference expression level for the at least five genes determines the subject has an acute rejection response.

51. The method of claim 49, wherein the statistical difference between the gene expression level and the reference expression level for the at least five genes determines the subject does not have an acute rejection response.

52. The method of claim 48, wherein the reference expression level is obtained from a control sample from at least one subject without an acute rejection response to a solid organ allograft.

53. The method of claim 52, wherein the statistical similarity between the gene expression level and the reference expression level for the at least five genes determines the subject does not have an acute rejection response.

54. The method of claim 52, wherein the statistical difference between the gene expression level and the reference expression level for the at least five genes determines the subject has an acute rejection response.

55. The method as in any one of claims 48-54, wherein the sample is a biological sample.

56. The method of claim 55, wherein the biological sample is selected from the group consisting of: a blood sample, a biopsy sample, a saliva sample, a cerebrospinal fluid sample, or a urine sample.

57. The method of claim 55, wherein the biological sample comprises peripheral blood leukocytes.

58. The method of claim 55, wherein the biological sample comprises peripheral blood mononuclear cells.

59. The method of claim 55, wherein the biological sample is a bronchoalveolar lavage sample.

60. The method as in any one of claims 48-59, wherein the solid organ allograft is one or more selected from the group consisting of: heart, lung, large intestine, small intestine, liver, kidney, pancreas, stomach, and bladder.

61. The method as in any one of claims 48-60, wherein the step of detecting comprises assaying the sample for an expression product of the at least ten genes.

62. The method of claim 61, wherein the expression product is a nucleic acid transcript.

63. The method of claim 61, wherein the expression product is a protein.

64. The method as in any one of claims 48-60, wherein the step of detecting comprises assaying the expression of the at least ten genes by hybridizing nucleic acids to oligonucleotide probes, by RT-PCR or by direct mRNA capture.

65. The method as in any one of claims 48-60, wherein the step of detecting comprises assaying the expression of the at least ten genes on one or more of: an array, a bead, and a nanoparticle.

66. The method as in any one of claims 48-65, wherein the subject has an acute rejection score of Grade 0, Grade 1A, Grade 1B, Grade 2, Grade 3A, Grade 3B, or Grade 4.

67. The method as in any one of claims 48-66, wherein the comparing step allows monitoring the progression of an acute rejection with equal to or greater than 70% sensitivity.

68. The method as in any one of claims 48-67, wherein the comparing step allows monitoring the progression of an acute rejection with equal to or greater than 70% specificity.

69. The method as in any one of claims 48-68, wherein the comparing step allows monitoring the progression of an acute rejection with equal to or greater than 70% positive predictive value (ppv).

70. The method as in any one of claims 48-69, wherein the comparing step allows monitoring the progression of an acute rejection with equal to or greater than 70% negative predictive value (npv).

71. A method for identifying a subject who has received a solid organ allograft in need of treatment of an acute rejection response, the method comprising:
a) detecting a gene expression level for at least ten genes in a sample from the subject, wherein the at least ten genes comprise CFLAR, DUSP1, IFNGR1, ITGAX, MAPK9, NAMPT, NKTR, PSEN1, RNF130, and RYBP;
b) comparing the gene expression level to a reference expression level of the at least ten genes; and
c) determining whether the subject has an acute rejection response based upon a statistical difference or a statistical similarity between the gene expression level and the reference expression level of at least five genes selected from the group consisting of CFLAR, DUSP1, IFNGR1, ITGAX, MAPK9, NAMPT, NKTR, PSEN1, RNF130, and RYBP, thereby identifying the subject in need of treatment of an acute rejection response.

72. The method of claim 71, wherein the reference expression level is obtained from a control sample from at least one subject with an acute rejection response to a solid organ allograft.

73. The method of claim 72, wherein the statistical similarity between the gene expression level and the reference expression level for the at least five genes identifies the subject in need of treatment for an acute rejection response.

74. The method of claim 72, wherein the statistical difference between the gene expression level and the reference expression level for the at least five genes identifies the subject as not requiring treatment for an acute rejection response.

75. The method of claim 71, wherein the reference expression level is obtained from a control sample from at least one subject without an acute rejection response to a solid organ allograft.

76. The method of claim 75, wherein the statistical similarity between the gene expression level and the reference expression level for the at least five genes identifies the subject as not requiring treatment for an acute rejection response.

77. The method of claim 75, wherein the statistical difference between the gene expression level and the reference expression level for the at least five genes identifies the subject in need of treatment for an acute rejection response.

78. The method as in any one of claims 71-77, wherein the sample is a biological sample.

79. The method of claim 78, wherein the biological sample is selected from the group consisting of: a blood sample, a biopsy sample, a saliva sample, a cerebrospinal fluid sample, or a urine sample.

80. The method of claim 78, wherein the biological sample comprises peripheral blood leukocytes.

81. The method of claim 78, wherein the biological sample comprises peripheral blood mononuclear cells.

82. The method of claim 78, wherein the biological sample is a bronchoalveolar lavage sample.

83. The method as in any one of claims 71-82, wherein the solid organ allograft is one or more selected from the group consisting of: heart, lung, large intestine, small intestine, liver, kidney, pancreas, stomach, and bladder.

84. The method as in any one of claims 71-83, wherein the step of detecting comprises assaying the sample for an expression product of the at least ten genes.

85. The method of claim 84, wherein the expression product is a nucleic acid transcript.

86. The method of claim 84, wherein the expression product is a protein.

87. The method as in any one of claims 71-83, wherein the step of detecting comprises assaying the expression of the at least ten genes by hybridizing nucleic acids to oligonucleotide probes, by RT-PCR or by direct mRNA capture.

88. The method as in any one of claims 71-83, wherein the step of detecting comprises assaying the expression of the at least ten genes on one or more of: an array, a bead, and a nanoparticle.

89. The method as in any one of claims 71-88, wherein the subject has an acute rejection score of Grade 0, Grade 1A, Grade 1B, Grade 2, Grade 3A, Grade 3B, or Grade 4.

90. The method as in any one of claims 71-89, wherein the comparing step identifies a subject who has received a solid organ allograft for treatment of an acute rejection response with equal to or greater than 70% sensitivity.

91. The method as in any one of claims 71-90, wherein the comparing step identifies a subject who has received a solid organ allograft for treatment of an acute rejection response with equal to or greater than 70% specificity.

92. The method as in any one of claims 71-91, wherein the comparing step identifies a subject who has received a solid organ allograft for treatment of an acute rejection response with equal to or greater than 70% positive predictive value (ppv).

93. The method as in any one of claims 71-92, wherein the comparing step identifies a subject who has received a solid organ allograft for treatment of an acute rejection response with equal to or greater than 70% negative predictive value (npv).

94. A method for treating an acute rejection (AR) response in a subject who has received a solid organ allograft, the method comprising:
a) detecting a gene expression level of at least ten genes in a sample from the subject, wherein the at least ten genes comprise CFLAR, DUSP1, IFNGR1, ITGAX, MAPK9, NAMPT, NKTR, PSEN1, RNF130, and RYBP;
b) comparing the gene expression level to a reference expression level of the at least ten genes;
c) determining the subject has an acute rejection response based upon a statistical difference or a statistical similarity between the gene expression level and the reference expression level of at least five genes selected from the group consisting of CFLAR, DUSP1, IFNGR1, ITGAX, MAPK9, NAMPT, NKTR, PSEN1, RNF130, and RYBP; and
d) administering a therapeutically effective amount of one or more of a therapeutic agent to treat the acute rejection response.

95. The method of claim 94, wherein the reference expression level is obtained from a control sample from at least one subject with an acute rejection response to a solid organ allograft.

96. The method of claim 95, wherein the statistical similarity between the gene expression level and the reference expression level for the at least five genes determines the subject has an acute rejection response.

97. The method of claim 94, wherein the reference expression level is obtained from a control sample from at least one subject without an acute rejection response to a solid organ allograft.

98. The method of claim 97, wherein the statistical difference between the gene expression level and the reference expression level for the at least five genes determines the subject has an acute rejection response.

99. The method as in any one of claims 94-98, wherein the sample is a biological sample.

100. The method of claim 99, wherein the biological sample is selected from the group consisting of: a blood sample, a biopsy sample, a saliva sample, a cerebrospinal fluid sample, or a urine sample.

101. The method of claim 99, wherein the biological sample comprises peripheral blood leukocytes.

102. The method of claim 99, wherein the biological sample comprises peripheral blood mononuclear cells.

103. The method of claim 99, wherein the biological sample is a bronchoalveolar lavage sample.

104. The method as in any one of claims 94-103, wherein the solid organ allograft is one or more selected from the group consisting of: heart, lung, large intestine, small intestine, liver, kidney, pancreas, stomach, and bladder.

105. The method as in any one of claims 94-104, wherein the step of detecting comprises assaying the sample for an expression product of the at least ten genes.

106. The method of claim 105, wherein the expression product is a nucleic acid transcript.

107. The method of claim 105, wherein the expression product is a protein.

108. The method as in any one of claims 94-104, wherein the step of detecting comprises assaying the expression of the at least ten genes by hybridizing nucleic acids to oligonucleotide probes, by RT-PCR or by direct mRNA capture.

109. The method as in any one of claims 94-104, wherein the step of detecting comprises assaying the expression of the at least ten genes on one or more of: an array, a bead, and a nanoparticle.

110. The method as in any one of claims 94-109, wherein the subject has an acute rejection score of Grade 0, Grade 1A, Grade 1B, Grade 2, Grade 3A, Grade 3B, or Grade 4.

111. The method as in any one of claims 94-110, wherein the comparing step aids in determining the subject has an acute rejection response with equal to or greater than 70% sensitivity.

112. The method as in any one of claims 94-111, wherein the comparing step aids in determining the subject has an acute rejection response with equal to or greater than 70% specificity.

113. The method as in any one of claims 94-112, wherein the comparing step aids in determining the subject has an acute rejection response with equal to or greater than 70% positive predictive value (ppv).

114. The method as in any one of claims 94-113, wherein the comparing step aids in determining the subject has an acute rejection response with equal to or greater than 70% negative predictive value (npv).

115. A method of treatment of an acute rejection in a subject who has received a solid organ allograft, comprising ordering a test comprising:
a) detecting a gene expression level of at least ten genes in a sample from the subject, wherein the at least ten genes comprise CFLAR, DUSP1, IFNGR1, ITGAX, MAPK9, NAMPT, NKTR, PSEN1, RNF130, and RYBP;
b) comparing the gene expression level to a reference expression level of the at least ten genes;
c) determining the subject has an acute rejection response based upon a statistical difference or a statistical similarity between the gene expression level and the reference expression level of at least five genes selected from the group consisting of CFLAR, DUSP1, IFNGR1, ITGAX, MAPK9, NAMPT, NKTR, PSEN1, RNF130, and RYBP; and
d) increasing the administration of a therapeutically effective amount of one or more of a therapeutic agent in a subject with an acute rejection response, maintaining the administration of a therapeutically effective amount of one or more of a therapeutic agent in a subject without an acute rejection response, or decreasing the administration of a therapeutically effective amount of one or more of a therapeutic agent in a subject without an acute rejection response.

116. The method of claim 115, wherein the reference expression level is obtained from a control sample from at least one subject with an acute rejection response to a solid organ allograft.

117. The method of claim 116, wherein the statistical similarity between the gene expression level and the reference expression level for the at least five genes determines the subject has an acute rejection response.

118. The method of claim 115, wherein the reference expression level is obtained from a control sample from at least one subject without an acute rejection response to a solid organ allograft.

119. The method of claim 117, wherein the statistical difference between the gene expression level and the reference expression level for the at least five genes determines the subject has an acute rejection response.

120. The method as in any one of claims 115-119, wherein the sample is a biological sample.

121. The method of claim 120, wherein the biological sample is selected from the group consisting of: a blood sample, a biopsy sample, a saliva sample, a cerebrospinal fluid sample, or a urine sample.

122. The method of claim 120, wherein the biological sample comprises peripheral blood leukocytes.

123. The method of claim 120, wherein the biological sample comprises peripheral blood mononuclear cells.

124. The method of claim 120, wherein the biological sample is a bronchoalveolar lavage sample.

125. The method as in any one of claims 115-124, wherein the solid organ allograft is one or more selected from the group consisting of: heart, lung, large intestine, small intestine, liver, kidney, pancreas, stomach, and bladder.

126. The method as in any one of claims 115-125, wherein the step of detecting comprises assaying the sample for an expression product of the at least ten genes.

127. The method of claim 126, wherein the expression product is a nucleic acid transcript.

128. The method of claim 126, wherein the expression product is a protein.

129. The method as in any one of claims 115-125, wherein the step of detecting comprises assaying the expression of the at least ten genes by hybridizing nucleic acids to oligonucleotide probes, by RT-PCR or by direct mRNA capture.

130. The method as in any one of claims 115-125, wherein the step of detecting comprises assaying the expression of the at least ten genes on one or more of: an array, a bead, and a nanoparticle.

131. The method as in any one of claims 115-130, wherein the subject has an acute rejection score of Grade 0, Grade 1A, Grade 1B, Grade 2, Grade 3A, Grade 3B, or Grade 4.

132. The method as in any one of claims 115-131, wherein the comparing step aids in determining the subject has an acute rejection response with equal to or greater than 70% sensitivity.

133. The method as in any one of claims 115-132, wherein the comparing step aids in determining the subject has an acute rejection response with equal to or greater than 70% specificity.

134. The method as in any one of claims 115-133, wherein the comparing step aids in determining the subject has an acute rejection response with equal to or greater than 70% positive predictive value (ppv).

135. The method as in any one of claims 115-134, wherein the comparing step aids in determining the subject has an acute rejection response with equal to or greater than 70% negative predictive value (npv).

136. A method for preparing a gene expression profile indicative of an acute rejection response to a solid organ allograft, the method comprising:
a) obtaining a gene expression product from a sample of at least one subject who has received a solid organ allograft and has an acute rejection response;
b) detecting the expression of at least ten genes, wherein the at least ten genes comprise CFLAR, DUSP1, IFNGR1, ITGAX, MAPK9, NAMPT, NKTR, PSEN1, RNF130, and RYBP; and
c) determining the expression level for at least five genes selected from the group consisting of CFLAR, DUSP1, IFNGR1, ITGAX, MAPK9, NAMPT, NKTR, PSEN1, RNF130, and RYBP, thereby preparing the gene expression profile indicative of an acute rejection response.

137. A method for preparing a gene expression profile indicative of an absence of an acute rejection response to a solid organ allograft, the method comprising:
a) obtaining a gene expression product from a sample of at least one subject who has received a solid organ allograft and does not have an acute rejection response;
b) detecting the expression of at least ten genes, wherein the at least ten genes comprise CFLAR, DUSP1, IFNGR1, ITGAX, MAPK9, NAMPT, NKTR, PSEN1, RNF130, and RYBP; and
c) determining the expression level for at least five genes selected from the group consisting of CFLAR, DUSP1, IFNGR1, ITGAX, MAPK9, NAMPT, NKTR, PSEN1, RNF130, and RYBP, thereby preparing the gene expression profile indicative of the absence of an acute rejection response.

138. The method as in any one of claims 136 and 137, wherein the sample is a biological sample.

139. The method of claim 138, wherein the biological sample is selected from the group consisting of: a blood sample, a biopsy sample, a saliva sample, a cerebrospinal fluid sample, or a urine sample.

140. The method of claim 138, wherein the biological sample comprises peripheral blood leukocytes.

141. The method of claim 138, wherein the biological sample comprises peripheral blood mononuclear cells.

142. The method of claim 138, wherein the biological sample is a bronchoalveolar lavage sample.

143. The method as in any one of claims 136-142, wherein the solid organ allograft is one or more selected from the group consisting of: heart, lung, large intestine, small intestine, liver, kidney, pancreas, stomach, and bladder.

144. The method as in any one of claims 136-143, wherein the step of detecting comprises assaying the sample for an expression product of the at least ten genes.

145. The method of claim 144, wherein the expression product is a nucleic acid transcript.

146. The method of claim 144, wherein the expression product is a protein.

147. The method as in any one of claims 136-143, wherein the step of detecting comprises assaying the expression of the at least ten genes by hybridizing nucleic acids to oligonucleotide probes, by RT-PCR or by direct mRNA capture.

148. The method as in any one of claims 136-143, wherein the step of detecting comprises assaying the expression of the at least ten genes on one or more of: an array, a bead, and a nanoparticle.

149. The method as in any one of claims 136-148, wherein the subject has an acute rejection score of Grade 0, Grade 1A, Grade 1B, Grade 2, Grade 3A, Grade 3B, or Grade 4.

150. A method for analysis of gene expression data obtained from a subject who has received a solid organ allograft for determination of an acute rejection response, the method comprising:
a) detecting the expression level for at least ten genes in a sample from the subject, wherein the at least ten genes comprise CFLAR, DUSP1, IFNGR1, ITGAX, MAPK9, NAMPT, NKTR, PSEN1, RNF130, and RYBP, thereby obtaining gene expression data from the subject;
b) comparing the gene expression data to a gene expression profile prepared by the method of claim 115 or 116; and
c) determining a statistical difference or a statistical similarity between the gene expression data and the gene expression profile of at least five genes selected from the group consisting of CFLAR, DUSP1, IFNGR1, ITGAX, MAPK9, NAMPT, NKTR, PSEN1, RNF130, and RYBP.

151. The method of claim 150, wherein the statistical similarity between the gene expression data and the gene expression profile prepared by the method of claim 115 for the at least five genes determines the subject will have an acute response.

152. The method of claim 150, wherein the statistical difference between the gene expression data and the gene expression profile prepared by the method of claim 115 for the at least five genes determines the subject will not have an acute response.

153. The method of claim 150, wherein the statistical similarity between the gene expression data and the gene expression profile prepared by the method of claim 116 for the at least five genes determines the subject will not have an acute response.

154. The method of claim 150, wherein the statistical difference between the gene expression data and the gene expression profile prepared by the method of claim 116 for the at least five genes determines the subject will have an acute response.

155. The method as in any one of claims 150-154, wherein the sample is a biological sample.

156. The method of claim 155, wherein the biological sample is selected from the group consisting of: a blood sample, a biopsy sample, a saliva sample, a cerebrospinal fluid sample, or a urine sample.

157. The method of claim 155, wherein the biological sample comprises peripheral blood leukocytes.

158. The method of claim 155, wherein the biological sample comprises peripheral blood mononuclear cells.

159. The method of claim 155, wherein the biological sample is a bronchoalveolar lavage sample.

160. The method as in any one of claims 150-159, wherein the solid organ allograft is one or more selected from the group consisting of: heart, lung, large intestine, small intestine, liver, kidney, pancreas, stomach, and bladder.

161. The method as in any one of claims 150-160, wherein the step of detecting comprises assaying the sample for an expression product of the at least ten genes.

162. The method of claim 161, wherein the expression product is a nucleic acid transcript.

163. The method of claim 161, wherein the expression product is a protein.

164. The method as in any one of claims 150-160, wherein the step of detecting comprises assaying the expression of the at least ten genes by hybridizing nucleic acids to oligonucleotide probes, by RT-PCR or by direct mRNA capture.

165. The method as in any one of claims 150-160, wherein the step of detecting comprises assaying the expression of the at least ten genes on one or more of: an array, a bead, and a nanoparticle.

166. The method as in any one of claims 150-165, wherein the subject has an acute rejection score of Grade 0, Grade 1A, Grade 1B, Grade 2, Grade 3A, Grade 3B, or Grade 4.

167. The method as in any one of claims 150-166, wherein the comparing step aids in the analysis of gene expression data for determination of acute rejection with equal to or greater than 70% sensitivity.

168. The method as in any one of claims 159-167, wherein the comparing step aids in the analysis of gene expression data for determination of acute rejection with equal to or greater than 70% specificity.

169. The method as in any one of claims 150-168, wherein the comparing step aids in the analysis of gene expression data for determination of acute rejection with equal to or greater than 70% positive predictive value (ppv).

170. The method as in any one of claims 150-169, wherein the comparing step aids in the analysis of gene expression data for determination of acute rejection with equal to or greater than 70% negative predictive value (npv).

171. The method as in any one of claims 150-170, wherein the expression level of the at least five genes is employed to predict the likelihood of an acute rejection response within 1 to 6 months of obtaining the sample.

172. A system for assessing an acute rejection response in a subject who has received a solid organ allograft, the system comprising:
a) a gene expression evaluation element for evaluating the expression level of at least ten genes in a sample from the subject to obtain gene expression data, wherein the at least ten genes comprise CFLAR, DUSP1, IFNGR1, ITGAX, MAPK9, NAMPT, NKTR, PSEN1, RNF130, and RYBP;
b) a phenotype determination element, wherein the phenotype determination element is one or more of (i) a gene expression profile indicative of an acute rejection response or (ii) a gene expression profile expression profile indicative of an absence of an acute rejection response; and
c) a comparison element for comparing the gene expression data to the gene expression profile of (i) and/or (ii), wherein the comparison element compares the expression of at least five genes selected from the group consisting of CFLAR, DUSP1, IFNGR1, ITGAX, MAPK9, NAMPT, NKTR, PSEN1, RNF130, and RYBP.

173. The system of claim 172, wherein the gene expression evaluation element comprises one or more of: a microarray chip, an array, a bead, and a nanoparticle.

174. The system of claim 172, wherein the gene expression evaluation element comprises at least one reagent for assaying the sample for an expression product of the at least ten genes.

175. The system of claim 174, wherein the expression product is a nucleic acid transcript.

176. The system of claim 174, wherein the expression product is a protein.

177. The system of claim 174, wherein the at least one reagent is an oligonucleotide of predetermined sequence that is specific for RNA encoded by the at least ten genes.

178. The system of claim 174, wherein the at least one reagent is an oligonucleotide of predetermined sequence that is specific for DNA complementary to RNA encoded by the at least 10 genes.

179. The system of claim 174, wherein the at least one reagent is an antibody specific for a gene expression product of the at least 10 genes.

180. The system of claim 172, wherein the phenotype determination element is computer-generated.

181. The system of claim 172, wherein comparison of said gene expression data to said gene expression profile is performed by a computer or an individual.

182. The system of claim 172, wherein a statistical similarity between the gene expression data and the gene expression profile of (i) for the at least five genes predicts the subject will have an acute rejection response.

183. The system of claim 172, wherein a statistical difference between the gene expression data and the gene expression profile of (i) for the at least five genes predicts the subject will not have an acute rejection response.

184. The system of claim 172, wherein a statistical similarity between the gene expression data and the gene expression profile of (ii) for the at least five genes predicts the subject will not have an acute rejection response.

185. The system of claim 182, wherein a statistical difference between the gene expression data and the gene expression profile of (ii) for the at least five genes predicts the subject will have an acute rejection response.

186. The system as in any one of claims 172-185, wherein the sample is a biological sample.

187. The system of claim 165, wherein the biological sample is selected from the group consisting of: a blood sample, a biopsy sample, a saliva sample, a cerebrospinal fluid sample, or a urine sample.

188. The system of claim 186, wherein the biological sample comprises peripheral blood leukocytes.

189. The system of claim 186, wherein the biological sample comprises peripheral blood mononuclear cells.

190. The system of claim 186, wherein the biological sample is a bronchoalveolar lavage sample.

191. The system as in any one of claims 172-190, wherein the solid organ allograft is one or more selected from the group consisting of: heart, lung, large intestine, small intestine, liver, kidney, pancreas, stomach, and bladder.

192. The system as in any one of claims 172-191, wherein the subject has a cardiac acute rejection score of Grade 0, Grade 1A, Grade 1B, Grade 2, Grade 3A, Grade 3B, or Grade 4.

193. The system as in any one of claims 172-192, wherein comparison of the gene expression data and the gene expression profile assesses an acute rejection response with equal to or greater than 70% sensitivity.

194. The system as in any one of claims 172-193, wherein comparison of the gene expression data and the gene expression profile assesses an acute rejection response with equal to or greater than 70% specificity.

195. The system as in any one of claims 172-194, wherein comparison of the gene expression data and the gene expression profile assesses an acute rejection response with equal to or greater than 70% positive predictive value (ppv).

196. The system as in any one of claims 172-195, wherein comparison of the gene expression data and the gene expression profile assesses an acute rejection response with equal to or greater than 70% negative predictive value (npv).

197. The system as in any one of claims 172-196, wherein the assessment of an acute rejection response in the subject predicts the likelihood of an acute rejection response within 1 to 6 months of obtaining the sample.

198. A kit for assessing an acute rejection response in a subject who has received a solid organ allograft, the kit comprising:
a) a gene expression evaluation element for evaluating the level of at least ten genes in a sample from the subject to obtain gene expression data, wherein the at least ten genes comprise CFLAR, DUSP1, IFNGR1, ITGAX, MAPK9, NAMPT, NKTR, PSEN1, RNF130, and RYBP;
b) a phenotype determination element, wherein the phenotype determination element is one or more of (i) a gene expression profile indicative of an acute rejection response or (ii) a gene expression profile expression profile indicative of an absence of an acute rejection response;
c) a comparison element for comparing the gene expression data to the gene expression profile of (i) and/or (ii), wherein the comparison element compares the expression of at least five genes selected from the group consisting of CFLAR, DUSP1, IFNGR1, ITGAX, MAPK9, NAMPT, NKTR, PSEN1, RNF130, and RYBP; and
d) a set of instructions for assessing acute rejection response in a subject who has received a solid organ allograft.

199. The kit of claim 198, wherein the gene expression evaluation element comprises one or more of a microarray chip, an array, a bead, and a nanoparticle.

200. The kit of claim 198, wherein the gene expression evaluation element comprises at least one reagent for assaying the sample for an expression product of the at least ten genes.

201. The kit of claim 200, wherein the expression product is a nucleic acid transcript.

202. The kit of claim 200, wherein the expression product is a protein.

203. The kit of claim 198, wherein the at least one reagent is an oligonucleotide of predetermined sequence that is specific for RNA encoded by the at least ten genes.

204. The kit of claim 198, wherein the at least one reagent is an oligonucleotide of predetermined sequence that is specific for DNA complementary to RNA encoded by the at least 10 genes.

205. The kit of claim 198, wherein the at least one reagent is an antibody specific for a gene expression product of the at least 10 genes.

206. The kit of claim 198, wherein the phenotype determination element is computer-generated.

207. The kit of claim 198, wherein comparison of said gene expression data to said gene expression profile is performed by a computer or an individual.

208. The kit of claim 198, wherein a statistical similarity between the gene expression data and the gene expression profile of (i) for the at least five genes predicts the subject will have an acute rejection response.

209. The kit of claim 198, wherein a statistical difference between the gene expression data and the gene expression profile of (i) for the at least five genes predicts the subject will not have an acute rejection response.

210. The kit of claim 198, wherein a statistical similarity between the gene expression data and the gene expression profile of (ii) for the at least five genes predicts the subject will not have an acute rejection response.

211. The kit of claim 198, wherein a statistical difference between the gene expression data and the gene expression profile of (ii) for the at least five genes predicts the subject will have an acute rejection response.

212. The kit as in any one of claims 198-211, wherein the sample is a biological sample.

213. The kit of claim 212, wherein the biological sample is selected from the group consisting of: a blood sample, a biopsy sample, a saliva sample, a cerebrospinal fluid sample, or a urine sample.

214. The kit of claim 212, wherein the biological sample comprises peripheral blood leukocytes.

215. The kit of claim 212, wherein the biological sample comprises peripheral blood mononuclear cells.

216. The kit of claim 212, wherein the biological sample is a bronchoaveolar lavage sample.

217. The kit as in any one of claims 198-216, wherein the solid organ allograft is one or more selected from the group consisting of: heart, lung, large intestine, small intestine, liver, kidney, pancreas, stomach, and bladder.

218. The kit as in any one of claims 198-217, wherein the subject has a cardiac acute rejection score of Grade 0, Grade 1A, Grade 1B, Grade 2, Grade 3A, Grade 3B, or Grade 4.

219. The kit as in any one of claims 198-218, wherein comparison of the gene expression data and the gene expression profile assesses an acute rejection response with equal to or greater than 70% sensitivity.

220. The kit as in any one of claims 198-219, wherein comparison of the gene expression data and the gene expression profile assesses an acute rejection response with equal to or greater than 70% specificity.

221. The kit as in any one of claims 198-220, wherein comparison of the gene expression data and the gene expression profile assesses an acute rejection response with equal to or greater than 70% positive predictive value (ppv).

222. The kit as in any one of claims 198-221, wherein comparison of the gene expression data and the gene expression profile assesses an acute rejection response with equal to or greater than 70% negative predictive value (npv).

223. The kit as in any one of claims 198-222, wherein the assessment of an acute rejection response in the subject predicts the likelihood of an acute rejection response within 1 to 6 months of obtaining the sample.
